SEQUENCE LISTING (23 sequences)

SEQ ID NO: 1 - DNA, 2307 bp, unidentified
  source 1..2307: mol_type = other DNA
gatccatgttggattgaaaaaaaatccccaaatccccatatgagtggacctacacagttcaaacgtgttgttcaagggtcaactgtgtatgaagactgctccatctgtggtggacagtgaggctttagaaagaggagacaggcaggctaatggatggtcttcatgggtgtgggaaaggaggagtggtcactgggcgcaggcagttggtctgtgggaccagtgaggcatgggctgtggctggcagtaggaaagcgggagaggctgagtgcagtggctcatgcctataatcccagcactttgggaagccaaggcaagaggcttgagcctgggaggtccaggctgcagtaagccataatcatgccactgcattctaggctgggcaacagaacaagaccttgtctcaaagacaacaaaacaaaacaaaaacggggagaggaaaggacccaagagatgttccaaaaaagaatttcaggaaacagtgaaaagttgcaaatggggcatcaggagccaggagcagagattaggtggcaggcagggggtgcagaaatgacaagttctgtctggaacagatgaaatttgaggtgcttgtgggacctttgagtgggcagaagcgggggctggagacaagggtgtgggaaaactgtgatgggggttgagggtggaaatcgggccggcagagcctccggggcagagaaagtcctatggggtgaccagatgagttgagaactgggtgggtgggtgcaggctcaatggaagcagaaactcatggatattgacttacatgggagaaggggaatggatgggggtcgcagtggggtggcagggctctcttttccttgtttttgtttttttgcccttccctcttctcttacttttccgtgagggctccccaggctcaggagagatcagggtggaagggtgacggccaaaccagggaaggctccaggtggctgaagcctggtctgtgcccagcagggccctggcgggctgttcctcactccaccgggcaggtgataactggtcagagctgcctccccacaggtgctggaggtaggctgggagggccggtgctccctgatgtggacagggggaggggtattgataagaggcgtggagagatccctagagatgcagtctgtggccctgggttccaacccagtgtgccaccacctggctgtgtgaccttcagcaagtgccattatttctctgagcctgtttgtttataaaatgaggaagagttggcacaagtttgaaaagatttctcaggctccacccggttctgaaattcggtaatttcccaactagggtgcactccccctgtaaggggctggggaggggacggtgcgaaaccaagttcagcttgtggagcggagccagagctgttggccgagcttgggcctggccaacgcctgccctcgggaaggtcctgtgtagggagactgcctggagggactaagcgagggctctaactgacgtctcaggggcagcctctcagcctgagaccctgctggggaagccgcgtcctgtgcactagctgcgcctagggctgaggtgagggcgcaggctccccagggtgtgtccgaattgcccgcctcagcccgcggcctgtcctgaccgcccagcagcggcagcagcgacaccctagggctccaggaagggcttgggaaggtgtaaaggcggggctaggctcggagggagccggagggaccgggcgcggttggctcccgggagcaactggagagtgaggagatcctcatccggggaagccccgcggccgcgtctctacagcgccccttctcgggctctggccctcccgctggttattctggacctgggggcccccagctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggaccagggaccaggctgagactcggggcgccagtccgggcaggggcagcggga

SEQ ID NO: 2 - DNA, 2204 bp, unidentified
  misc_feature 1..2204: note = Fragment pNR4A1(2204)
  source 1..2204: mol_type = other DNA
cagttcaaacgtgttgttcaagggtcaactgtgtatgaagactgctccatctgtggtggacagtgaggctttagaaagaggagacaggcaggctaatggatggtcttcatgggtgtgggaaaggaggagtggtcactgggcgcaggcagttggtctgtgggaccagtgaggcatgggctgtggctggcagtaggaaagcgggagaggctgagtgcagtggctcatgcctataatcccagcactttgggaagccaaggcaagaggcttgagcctgggaggtccaggctgcagtaagccataatcatgccactgcattctaggctgggcaacagaacaagaccttgtctcaaagacaacaaaacaaaacaaaaacggggagaggaaaggacccaagagatgttccaaaaaagaatttcaggaaacagtgaaaagttgcaaatggggcatcaggagccaggagcagagattaggtggcaggcagggggtgcagaaatgacaagttctgtctggaacagatgaaatttgaggtgcttgtgggacctttgagtgggcagaagcgggggctggagacaagggtgtgggaaaactgtgatgggggttgagggtggaaatcgggccggcagagcctccggggcagagaaagtcctatggggtgaccagatgagttgagaactgggtgggtgggtgcaggctcaatggaagcagaaactcatggatattgacttacatgggagaaggggaatggatgggggtcgcagtggggtggcagggctctcttttccttgtttttgtttttttgcccttccctcttctcttacttttccgtgagggctccccaggctcaggagagatcagggtggaagggtgacggccaaaccagggaaggctccaggtggctgaagcctggtctgtgcccagcagggccctggcgggctgttcctcactccaccgggcaggtgataactggtcagagctgcctccccacaggtgctggaggtaggctgggagggccggtgctccctgatgtggacagggggaggggtattgataagaggcgtggagagatccctagagatgcagtctgtggccctgggttccaacccagtgtgccaccacctggctgtgtgaccttcagcaagtgccattatttctctgagcctgtttgtttataaaatgaggaagagttggcacaagtttgaaaagatttctcaggctccacccggttctgaaattcggtaatttcccaactagggtgcactccccctgtaaggggctggggaggggacggtgcgaaaccaagttcagcttgtggagcggagccagagctgttggccgagcttgggcctggccaacgcctgccctcgggaaggtcctgtgtagggagactgcctggagggactaagcgagggctctaactgacgtctcaggggcagcctctcagcctgagaccctgctggggaagccgcgtcctgtgcactagctgcgcctagggctgaggtgagggcgcaggctccccagggtgtgtccgaattgcccgcctcagcccgcggcctgtcctgaccgcccagcagcggcagcagcgacaccctagggctccaggaagggcttgggaaggtgtaaaggcggggctaggctcggagggagccggagggaccgggcgcggttggctcccgggagcaactggagagtgaggagatcctcatccggggaagccccgcggccgcgtctctacagcgccccttctcgggctctggccctcccgctggttattctggacctgggggcccccagctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggacc

SEQ ID NO: 3 - DNA, 1750 bp, unidentified
  misc_feature 1..1750: note = Fragment pNR4A1(1750)
  source 1..1750: mol_type = other DNA
caggagcagagattaggtggcaggcagggggtgcagaaatgacaagttctgtctggaacagatgaaatttgaggtgcttgtgggacctttgagtgggcagaagcgggggctggagacaagggtgtgggaaaactgtgatgggggttgagggtggaaatcgggccggcagagcctccggggcagagaaagtcctatggggtgaccagatgagttgagaactgggtgggtgggtgcaggctcaatggaagcagaaactcatggatattgacttacatgggagaaggggaatggatgggggtcgcagtggggtggcagggctctcttttccttgtttttgtttttttgcccttccctcttctcttacttttccgtgagggctccccaggctcaggagagatcagggtggaagggtgacggccaaaccagggaaggctccaggtggctgaagcctggtctgtgcccagcagggccctggcgggctgttcctcactccaccgggcaggtgataactggtcagagctgcctccccacaggtgctggaggtaggctgggagggccggtgctccctgatgtggacagggggaggggtattgataagaggcgtggagagatccctagagatgcagtctgtggccctgggttccaacccagtgtgccaccacctggctgtgtgaccttcagcaagtgccattatttctctgagcctgtttgtttataaaatgaggaagagttggcacaagtttgaaaagatttctcaggctccacccggttctgaaattcggtaatttcccaactagggtgcactccccctgtaaggggctggggaggggacggtgcgaaaccaagttcagcttgtggagcggagccagagctgttggccgagcttgggcctggccaacgcctgccctcgggaaggtcctgtgtagggagactgcctggagggactaagcgagggctctaactgacgtctcaggggcagcctctcagcctgagaccctgctggggaagccgcgtcctgtgcactagctgcgcctagggctgaggtgagggcgcaggctccccagggtgtgtccgaattgcccgcctcagcccgcggcctgtcctgaccgcccagcagcggcagcagcgacaccctagggctccaggaagggcttgggaaggtgtaaaggcggggctaggctcggagggagccggagggaccgggcgcggttggctcccgggagcaactggagagtgaggagatcctcatccggggaagccccgcggccgcgtctctacagcgccccttctcgggctctggccctcccgctggttattctggacctgggggcccccagctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggacc

SEQ ID NO: 4 - DNA, 1251 bp, synthetic construct
  source 1..1251: mol_type = other DNA
caggtgataactggtcagagctgcctccccacaggtgctggaggtaggctgggagggccggtgctccctgatgtggacagggggaggggtattgataagaggcgtggagagatccctagagatgcagtctgtggccctgggttccaacccagtgtgccaccacctggctgtgtgaccttcagcaagtgccattatttctctgagcctgtttgtttataaaatgaggaagagttggcacaagtttgaaaagatttctcaggctccacccggttctgaaattcggtaatttcccaactagggtgcactccccctgtaaggggctggggaggggacggtgcgaaaccaagttcagcttgtggagcggagccagagctgttggccgagcttgggcctggccaacgcctgccctcgggaaggtcctgtgtagggagactgcctggagggactaagcgagggctctaactgacgtctcaggggcagcctctcagcctgagaccctgctggggaagccgcgtcctgtgcactagctgcgcctagggctgaggtgagggcgcaggctccccagggtgtgtccgaattgcccgcctcagcccgcggcctgtcctgaccgcccagcagcggcagcagcgacaccctagggctccaggaagggcttgggaaggtgtaaaggcggggctaggctcggagggagccggagggaccgggcgcggttggctcccgggagcaactggagagtgaggagatcctcatccggggaagccccgcggccgcgtctctacagcgccccttctcgggctctggccctcccgctggttattctggacctgggggcccccagctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggacc

SEQ ID NO: 5 - DNA, 734 bp, unidentified
  misc_feature 1..734: note = Fragment pNR4A1(734)
  source 1..734: mol_type = other DNA
tgtgcactagctgcgcctagggctgaggtgagggcgcaggctccccagggtgtgtccgaattgcccgcctcagcccgcggcctgtcctgaccgcccagcagcggcagcagcgacaccctagggctccaggaagggcttgggaaggtgtaaaggcggggctaggctcggagggagccggagggaccgggcgcggttggctcccgggagcaactggagagtgaggagatcctcatccggggaagccccgcggccgcgtctctacagcgccccttctcgggctctggccctcccgctggttattctggacctgggggcccccagctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggacc

SEQ ID NO: 6 - DNA, 35 bp, unidentified
  misc_feature 1..35: note = -2095 PrNR4A1 EcoRI
  source 1..35: mol_type = other DNA
gatacgaattccagttcaaacgtgttgttcaaggg

SEQ ID NO: 7 - DNA, 33 bp, unidentified
  misc_feature 1..33: note = 1641 PrNR4A1 EcoRI
  source 1..33: mol_type = other DNA
gatacgaattccaggagcagagattaggtggca

SEQ ID NO: 8 - DNA, 34 bp, unidentified
  misc_feature 1..34: note = -1142 PrNR4A1 EcoRI
  source 1..34: mol_type = other DNA
gatacgaattccaggtgataactggtcagagctg

SEQ ID NO: 9 - DNA, 30 bp, unidentified
  misc_feature 1..30: note = -625 PrNR4A1 EcoRI
  source 1..30: mol_type = other DNA
gatacgaattctgtgcactagctgcgccta

SEQ ID NO: 10 - DNA, 26 bp, unidentified
  misc_feature 1..26: note = ∩PrNR4A1 EcoRI
  source 1..26: mol_type = other DNA
gatacggatccggtcccgcgtgcgct

SEQ ID NO: 11 - DNA, 11573 bp, unidentified
  misc_feature 1..11573: note = FAM0-OVA construction
  misc_feature 561..3149: note = /gene="Nucleic acid sequence from the nucleotide in position 561to the nucleotide in position 3149 coding for the variableregions of the monoclonal antibody directed against the ovalbumin(OVA) protein "
  source 1..11573: mol_type = other DNA
agcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattctagcatttaaatagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggcgcgccctggacaccccgcagagggtggccctaggccccctgtccgatcatgttcctgtagtcggggatgatggtctgcttcaggtccaccaccgaggagaagatccacttcaccttgaagaaggtgacggtggcactgtagcacacgcttaacaggaagagtgtgatgaagatggtgatggtcgtccacagcccgtccagctccccgtcctgcgcctccgcacagctctcctccagttgcagctccggagacagggagaggctcttctgtgtgtagtggttgtgcagagcctcatgcatcacggagcatgagaagacgttcccctgctgccacctgctcttgtccacggtgagttttgagtagaggaagaaggagccgtcggagtccagcacgggaggcgtggtcttgtagttgttctccggctgcccattgctctcccactccacggcgatgtcgctgggatagaagcctttgaccaggcaggtcaggctgacttgattcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtcaacataccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtgctagctgaagagacagtgaccagagtcccttggccccagtatttactcccccttgaacagtaatagaccgcagagtcctcagatgtcagactgatgagctgaatgtaggctatattggaggacttgtcaacagtcaatgtggccttgtctttgaacatttgattgtagtgagtttttctgtctgaacaatcaatcataccaatccattcaaggccttgtccaggcctctgtttcacccaattcatccagtaactggtgaagatgtagccagaagccttacaggacagcttcactgaagccccaggcctcaccaacacagccccaggctgctgcagttggacctgggagtggactgtagctgttgctaccaaaaagaggatgatacagctccatcccatgggccctgggttggactccacgtctcccgccaacttgagaaggtcaaaattcaaagtctgtttcaccggtgctctcttagcccgacactctcccctattgaagctctttgtgacgggcgaactcaggccctgatgggtgacttcgcacgcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtcttacgtttgatttccaacttggtacctccaccgaacgtctgaggaaaatgtgtagcttgccagcaataataaactcccaaatcctcagcctccactctgctgattttcagtgtgaaatccgttccagacccactgccactgaacctgtcagggactccagagtccagtttagacaccagatagattaggcgctttggagactggcctggcctctgtaacaaccaattcaaatatgtctttccatcactatctaagaggctctgacttgacttgcaagagatggaggctggttgtccaatggtaaccgacaaagtgagtggagtctgggtcatcacaacatcaccgttggtccgaatccagagcactaacagaaacaggaactgggcaggactcatcatggcggcggcgaattcaccggtacggggtggagatccgagctcggtaccaagcttcgtagctgtgtcctgactgagtctgactcctgcacaaagtgtgaccagcctattaataaggcttcagggcaggaggttgtgctctgggaacatgcaaatgagcaggggatggggcaggctgggcacagctgcacgtacgggtcaactgtaatcttggccacctgcctaagaggaagtggctagcttcacttctgaccctcagcaactgccaggtggcctcttggaaatccccctctgggggattccacccgttgggtgggagaggctgagccctatcaagttgttgcagaccaaaataatctccttaaatatcacttttgagatcagctggggtaaacgacagcaacacaatgacaaatcattaaactattttagagattatgaaattaaaatactcagattaaaattttcctatcacagaattaaggtactggaaaatatgtttaagtttttattaatcacattgctataggtttagatattttgtacaactgaaataaaatcacacactggcagctacatttttgaaagttaaaaacatggtcacgaatatatcttattttaaaatcagttaatataccttaatggtatttaatgccaaattcaaagtgaattgatcaagccctcagtggccaggtcatgggtgtgatttttactctggctgagccgtacgaccggtgaaagggtgtggagtgctccacccagccctgttggaggaggatgggagcgaaagcaacagatgtgctcaaggttctgttttcagttccccaaagggtcttctccttgaccaaagcagtgtgacggttgctatgacagttgctatcttggtccctgggactctccctcataaggtagcattactttttttaatcttctcaggcaagtaaccatgaaactcaccggtttagatcttgggtgggttaattaaccatctttatggctcgagatgttaaactgtacaagtaaagcggccgcgactctagtccctcccaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctgcattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacaca

SEQ ID NO: 12 - DNA, 20 bp, unidentified
  source 1..20: mol_type = other DNA
tgtgtgcccgtctgttgtgt

SEQ ID NO: 13 - DNA, 20 bp, unidentified
  source 1..20: mol_type = other DNA
gagtcctgcgtcgagagagc

SEQ ID NO: 14 - DNA, 20 bp, unidentified
  source 1..20: mol_type = other DNA
cagtggcgcccgaacaggga

SEQ ID NO: 15 - DNA, 22 bp, unidentified
  source 1..22: mol_type = other DNA
tccgtgtggatcggcggctcca

SEQ ID NO: 16 - DNA, 21 bp, unidentified
  source 1..21: mol_type = other DNA
ctgcttgctgatccacatctg

SEQ ID NO: 17 - DNA, 24 bp, unidentified
  source 1..24: mol_type = other DNA
cctggcctcgctgtccaccttcca

SEQ ID NO: 18 - DNA, 54 bp, synthetic construct
  source 1..54: mol_type = other DNA
gagggcagaggaagtcttctaacatgcggtgacgtggaggagaatcccggccct

SEQ ID NO: 19 - DNA, 36 bp, synthetic construct
  source 1..36: mol_type = other DNA
gatcgaattccaggaagggcttgggaaggtgtaaag

SEQ ID NO: 20 - DNA, 29 bp, synthetic construct
  source 1..29: mol_type = other DNA
gatcgaattcagctgggacccgagtccgg

SEQ ID NO: 21 - DNA, 33 bp, synthetic construct
  source 1..33: mol_type = other DNA
gatcgaattcttgtatggccaaagctcgacggg

SEQ ID NO: 22 - DNA, 608 bp, synthetic construct
  source 1..608: mol_type = other DNA
caggaagggcttgggaaggtgtaaaggcggggctaggctcggagggagccggagggaccgggcgcggttggctcccgggagcaactggagagtgaggagatcctcatccggggaagccccgcggccgcgtctctacagcgccccttctcgggctctggccctcccgctggttattctggacctgggggcccccagctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggacc

SEQ ID NO: 23 - DNA, 415 bp, synthetic construct
  source 1..415: mol_type = other DNA
agctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggacc

(19) United States
(12) Patent Application Publication
(10) Pub. No.: US 2025/0388932 A1
(43) Pub. Date: Dec. 25, 2025

(54) NUCLEIC ACID SYSTEM TO SPECIFICALLY REPROGRAM B AND T CELLS AND USES THEREOF

(71) Applicants: INSTITUT NATIONAL DE LA SANTE ET DE LA RECHERCHE MEDICALE, Paris (FR); CENTRE NATIONAL DE LA RECHERCHE SCIENTIFIQUE, Paris (FR); ECOLE NORMALE SUPÉRIEURE DE LYON, Lyon (FR); UNIVERSITE CLAUDE BERNARD LYON 1, Villeurbanne (FR)

(72) Inventors: Floriane FUSIL, Lyon (FR); Audrey PAGE, Lyon (FR); François-Loïc COSSET, Lyon Cedex 07 (FR)

(21) Appl. No.: 18/841,830

(22) PCT Filed: Mar. 10, 2023
(86) PCT No.: PCT/EP2023/056228

§ 371 Date: Aug. 27, 2024

(30) Foreign Application Priority Data
Mar. 11, 2022 (EP) 22305277.0

(51) Int. Cl. C12N 15/867; A61K 31/7088; A61K 48/00; C07K 14/725; C12N 5/0781; C12N 5/0783; C12N 5/10; C12N 15/62

(52) U.S. Cl. CPC C12N 15/867; A61K 31/7088; A61K 48/005; C07K 14/7051; C12N 5/0635; C12N 5/0636; C12N 5/10; C12N 15/62; C07K 2319/02; C07K 2319/03; C12N 2510/00; C12N 2740/10043; C12N 2740/10052

(57) ABSTRACT

The invention relates to a nucleic acid system comprising a sensor component and a transducer/effector component that allows to specifically reprogram B and T cells. The inventors successfully demonstrated that upon binding of target molecules on the dedicated sensor (targeting a given pathological signal), the transducer pNR4A1 was specifically activated leading to the expression of the effector therapeutic molecules placed under its control. The main advantage of this system is its full programmability in terms of recognized signal and output functions, which can be adapted to the targeted diseases. As such, the invention may be applied to many pathologies, such as tumors, auto immune disorders, transplantation rejection, allergies, neurological disorders, and infectious diseases.

### SEQUENCE LISTING

This application includes as the Sequence Listing the complete contents of the xml file “13390151_sequence_listing_v2”, created Jul. 30, 2025, containing 46,142 bytes, hereby incorporated by reference.

### TECHNICAL FIELD

The invention relates to the field of synthetic immunology and in particular to a new synthetic circuit which may be implemented in B or T cells by lentiviral vector transduction in order to reprogram the cells. It further relates to the use of said synthetic circuit and lentiviral vectors in the treatment of tumors, immune disorders, transplantation rejection, allergies, neurological disorders and infectious diseases.

### TECHNICAL BACKGROUND

The expansion of techniques for genetic engineering has recently brought a new dimension for synthetic biology approaches. Synthetic biology relies on the design of genetic parts and biological blocks that are assembled in the target cells to create new gene networks (Sedlmayer et al.). For instance, synthetic bio-sensing circuits are composed of sensor elements, that bind the signal molecule and transducer modules, which will lead to specific cellular responses. The input or “inducing signal” should be specific of the disease, such as a specific antigen or a dys-regulation of the microenvironment, and should be recognized by a dedicated receptor. This recognition should trigger a signaling cascade and the integration of information, leading or not to an output response from the reprogrammed cell. Such circuits are of valuable importance from a therapeutic point of view notably for apoptosis induction or regulated drug delivery in vivo.

This kind of approach has been implemented in immune cells, and represents a new field called ‘synthetic immunology’, which holds great promises for the treatment of many diseases as cells of the immune system play a crucial role in detecting and responding to pathological deviations (Roybal et al.). Several new capacities can be conferred by the genetic reprogramming of immune cells to enhance their endogenous properties. Indeed, immune cells recognize pathological signals, such as pathogens, auto-antigens or inflammation and trigger responses to restore homeostasis, but their recognition capacity can be potentiated by adding new receptors to more precisely distinguish a pathological environment from a normal environment. Similarly, it is possible to play on their effector functions, by forcing the expression of therapeutic cytokines for instance.

Several characteristics of immune cells make them the perfect candidates for synthetic biology approaches.

First, these cells move freely in the body to patrol and infiltrate various tissues. Due to their global distribution in the body, immune cells can act as intermediaries and communicate with other cell types, thus leading to the modification of the immune response on a larger scale.

Second, they naturally expand when stimulated and they can differentiate in memory cells and thus persist on the long term, which will be particularly useful especially in case of disease flares.

In addition, they are involved in many diseases, such as cancers or autoimmune diseases, either because they cannot ensure their protective functions, or because they contribute to the disease in a pathological way.

Finally, these cells can be easily collected and modified. Most of synthetic immunology approaches used T cells, the best-known example being the successful chimeric antigen receptor (CAR) T cells that were notably developed in cancer immunotherapy approaches (Chabannon et al.).

However, B cells offer also unique opportunities for synthetic immunology. Paving the way, a first-in-man phase I/IIa clinical trial in patients was recently initiated to evaluate the safety and tolerability of adoptively transferred donor B-cells. B-cells were manufactured under GMP conditions and their transfer was well tolerated without any acute adverse reactions during the 4-month follow-up post adoptive transfer (Tittlbach et al.).

The immune system is involved in many diseases such as auto-immune diseases, where it attacks its own components or such as cancers where it no longer recognises cancer cells as abnormal.

According to the condition to be treated, it is advantageous to specifically be able to either stimulate or suppress an immune response in an individual.

Immunostimulation or immune stimulation refers to the stimulation of the immune system by an external source. It aims at providing a protective effect, for example against microorganisms or tumors, by eliciting an immune reaction, in particular antibody production, either to increase the body's ability to fight the disease, or as a preventive measure. Vaccines and synthetic peptides have successfully been used in the past as immunostimulatory agents.

On the contrary, when the disease or condition stems from an ectopic activity of the immune system, such as in the case of autoimmune diseases or allergies, it is essential to be able to suppress the immune response. Deliberately inducing immunosuppression may also be performed to prevent the body from rejecting an organ transplant.

Current treatments do not allow treating most of the autoimmune diseases or certain cancers which are resistant to conventional therapies. Indeed, autoimmune disease treatments are based on total immunosuppression (in particular via corticosteroids) and do not restore tolerance towards “self” components. Thus, they are only able to address the symptoms of the disease, without treating the causes and are therefore inefficient in preventing relapses. Similarly, certain cancers are refractory to current treatments such as radiotherapies, chemotherapies or immunotherapies and new alternatives which lead to less side effects and act in a more targeted manner are necessary.

Therefore, there is a need for safe and improved treatments against diseases involving the immune system, in particular for treatments which allow total remission for the patients with minimal side effects, and preventing potential flares of the disease.

There is also a need for treatments against diseases involving the immune system which allow for an increase in the therapeutical potential of the B or T lymphocytes and for a patient-specific response.

Further, there is a need for treatments against diseases involving the immune system whose effect can be “turned on” or “turned off”, but also increased or reduced according to the pathogenic signal.

There is also a need for a safe cellular therapy system which may be easily adapted to treat various diseases involving the immune system, such as tumors, immune disorders, transplantation rejection, neurological disorders, allergies and infectious diseases.

The present invention has for purpose to satisfy all or part of those needs.

The present invention results from the development by the inventors of a cellular therapy system in the form of a nucleic acid system which aims at reprogramming, notably, B lymphocytes or T cells ex vivo via a vector, such as a lentiviral vector, which encodes a therapeutic synthetic circuit before re-injecting them in the patient.

The nucleic acid system of the invention encodes a sensor, which targets pathological ligands, especially pathological antigens, a transducer which is activated upon the binding of a target pathological ligand, especially of a target pathological antigen, to the sensor, and a transducer/effector component which produces therapeutical effector molecules upon activation of the transducer. Thus, the pathological ligand, especially the pathological antigen, recognition by the sensor will lead to the activation of the transducer and the regulated expression of the therapeutical effector molecules.

The terms “effector molecule” and “effector protein” may be used interchangeably herein. In particular, the effector protein is a therapeutical effector protein.

Said therapeutical effector proteins are then secreted by the reprogrammed blood cells, in particular by the reprogrammed B lymphocytes, also termed “reprogrammed B-cells”, which produce one or more therapeutical effector molecules after pathological ligand recognition, especially pathological antigen recognition. Consequently, once the targeted physiological disorder is overcome, the target pathological ligand, especially the pathological antigen, is normally no longer available and thus does no longer activate the nucleic acid system present in the reprogrammed blood cells, especially in the reprogrammed B cells, and the effector molecules produced upon activation of the said nucleic acid system are no longer produced and secreted by the said reprogrammed cells, especially by the said reprogrammed B cells. This nucleic acid system switch allows avoiding any systemic effects which are often seen in the current treatments (which are based on the ingestion or infusion of drugs) and will increase the molecules' efficiency which be more concentrated locally as a result of the “homing and proliferation” effect of the reprogrammed cells.

The aim is to increase the immunological properties of the patient's cells of the immune system, especially B lymphocytes, to cure a targeted disease or a targeted disorder.

One of the many advantages of this nucleic acid system resides in its complete programmability, which makes it adaptable to treating various diseases, by appropriately selecting the sensor and/or effector molecules. Another main advantage of the nucleic acid system is its signal-specific regulation combined to memory and long-term therapy.

The present invention thus concerns a nucleic acid system comprising:
(i) a sensor component comprising a sequence encoding
(a) at least one extracellular ligand recognition domain;
(b) a transmembrane domain of a B-cell receptor (BCR) or of a T-cell receptor (TCR); and
(c) optionally a signaling domain that controls cell activation of a B-cell receptor or of a T-cell receptor; and
(ii) a transducer/effector component comprising a sequence encoding
(d) the pNR4A1 promoter consisting of SEQ ID NO: 1, or a functional fragment thereof; and
(e) at least one effector protein of interest.

In some embodiments, the transducer/effector component comprises (d) a functional fragment of the pNR4A1 promoter (which may also be termed “NR4A1 promoter” herein). In particular, the functional fragment of the NR4A1 promoter has a length from 200 bp to 2210 bp, in particular from 500 bp to 2210 bp. In particular, the functional fragment of the NR4A1 promoter may have a nucleic acid sequence selected from a group consisting of SEQ ID NO:2, SEQ ID NO:3, SEQ ID NO:4, SEQ ID NO:5, SEQ ID NO: 22 and SEQ ID NO: 23, in particular consisting of SEQ ID NO:2, SEQ ID NO:3, SEQ ID NO:4 and SEQ ID NO:5, preferably may have the sequence of SEQ ID NO:5.

According to further advantages, the extracellular ligand recognition domain (a) the transmembrane domain (b) and, if present, the signaling domain (c) form a B-cell receptor, a T-cell receptor, a chimeric immune receptor (CIR), such as a CAR-T cell receptor, a CAR-NK cell receptor, a B-cell antibody receptor (BAR) or a chimeric autoantibody receptor T (CAAR-T) cell and especially form a B-cell receptor.

In some embodiments, the extracellular ligand recognition domain (a) comprises at least one ligand binding fragment which binds to a ligand of interest, in particular comprises a ligand binding fragment selected from ligand binding fragments derived from antibodies, i.e. ligand binding fragments comprising an antigen binding domain of an antibody, such as Fab fragments, Fab′ fragments, F(ab′)2 fragments; Fd fragments, CDRs,-containing fragments Fv fragments, scFVs, dsFvs, and sc(Fv)2; from antibody mimetics such as affibodies, affilins, affitins, adnectins, atrimers, evasins, DARPins, anticalins, avimers, fynomers, and versabodies; from aptamers, and mixtures thereof.

Advantageously, the ligand of interest is selected from one or more tumor antigens, one or more self-antigens, one or more allo-antigens, one or more viral antigens, one or more bacterial antigens, one or more allergen antigens, or one or more markers of neurological disorders.

In a particular embodiment, the at least one effector protein of interest is an immunostimulatory protein or an immunosuppressant protein. In particular, the at least one effector protein of interest is selected from pro-inflammatory cytokines, such as IL-18, gamma-interferon and TNF; anti-inflammatory cytokines, such as IL-10 and IL-4; co-stimulation molecules, such as CD80, CD86 and CD40; and inhibitor molecules, such as FasL and Fas.

According to another object, the present disclosure relates to a vector comprising a nucleic acid system as described herein. In particular, the vector is a retroviral vector, in particular is a lentiviral vector.

The disclosure further relates to a kit comprising:
a first vector comprising the sensor component of the nucleic acid system as described herein, and
a second vector comprising the transducer/effector component of the nucleic acid system as described herein.

According to another object, the present disclosure relates to a cell, in particular a B cell or a T cell, which has been transformed by a nucleic acid system, by a vector or by a kit as described herein.

The disclosure also relates to a pharmaceutical composition comprising a nucleic acid system, a vector comprising the sensor component, the transducer/effector component, or both, of the said nucleic acid system, a kit, or a cell as described herein, and a pharmaceutically acceptable vehicle.

Also, the disclosure relates to a method for preventing and/or treating a tumor, an immune disorder, a transplantation rejection, an allergy, a neurological disorder, and/or an infectious disease comprising at least a step of administering a nucleic acid system according, a vector, a kit, a cell, or a pharmaceutical composition as described herein to an individual in need thereof.

According to another embodiment, the disclosure relates to a nucleic acid system, a vector, a kit, or a cell as described herein for its use in the prevention and/or the treatment of a tumor, an immune disorder, a transplantation rejection, an allergy, a neurological disorder, and/or an infectious disease.

The disclosure further relates to a nucleic acid system, a vector, a kit, or a cell as described herein for use as a medicament.

The foregoing and other objects, features and advantages of the invention will be described in more detail by referring to the attached drawings, the following description, and the Examples provided hereafter.

### DESCRIPTION OF THE FIGURES

FIG. 1 illustrates the isolation of small ectopic BCR-inducible promoter constructs. (A) is a schematic representation of the lentiviral vector comprising the pNR4A1(2204) fragment (B) represents a schematic representation of the NR4A1 reporter constructs with putative binding domains of the NF-kB and NFAT transcription factors. Reporters with several sizes (2204, 1750, 1251 or 734 bp) were constructed. (C) shows the GFP induction of each reporter construct following 24 h of BCR stimulation. From the top down: pNR4A1(734)-GFP, pNR4A1(1251)-GFP, pNR4A1(1750)-GFP, pNR4A1(2204)-GFP and control SFFV-GFP. BJAB cells were transduced with lentiviral vectors encoding reporter constructions before stimulation with F(ab′)2 IgM (2.5 g/ml) or PMA (15 ng/ml) combined to ionomycin (1 M) during 24 h. Median GFP expression of GFP+ cells after stimulation was normalized by the non-stimulated condition. (D) Transduction efficiency of BJAB cells by the reporter constructions was assessed by the percentage of GFP+ cells following transduction for each construct four days after transduction (n=4, ANOVA 2 and multiple t-tests with Sidak Bonferroni collection (alpha=0.05)).

FIG. 2 shows the characterization and inducibility of the 734-bp NR4A1 reporter construct. (A) Dose response of the 734-bp NR4A1 reporter construct with increasing amounts of F(ab′)2 IgM. BJAB cells cere transduced with lentiviral vectors encoding the 734-bp inducible reporter of the SFFV constitutive reporter before 24 h of stimulation with F(ab′)2 IgM. GFP median expression of DAPI-GFP+ cells relative to unstimulated cells was assessed (n=4). (B) Specificity of the induction through BCR. BJAB cells were transduced with lentiviral vectors encoding the reporter constructs (734-bp NR4A1 or SFFV as a constitutive control) before stimulation with F(ab′)2 IgM (2.5 g/ml), F(ab)′ IgG (2.5 g/ml), LPS (10 μg/ml) or CpG (10 μg/ml) or PMA (15 ng/ml) combined to ionomycin (1 M) during 24 h. Median GFP expression of GFP+ cells after stimulation was normalized by the non-stimulated condition (n=4).

FIG. 3 represents the kinetics characterization of the inducible 734-bp NR4A1 promoter. (A) Kinetics of induction of the promoter. BJAB cells were transduced with lentiviral vectors encoding the TurboGFPdes under the 734-bp inducible reporter or the SFFV constitutive reporter before 0 to 24 h of stimulation with F(ab′)2 IgM or PMA combined with ionomycin. TurboGFPdes median expression in each condition (after the indicated stimulation duration in hours) was assessed and normalized by TurboGFPdes median of unstimulated cells (n=3). (B) Kinetics of extinction of the promoter. BJAB cells were transduced with lentiviral vectors encoding the TurboGFPdes under the 734-bp inducible reporter or the SFFV constitutive reporter before 0, 1, 4, 8 and 24 h of stimulation with F(ab′)2 IgM or PMA combined with ionomycin. Cells were then washed 3 times and put in culture. TurboGFPdes median expression was assessed at day 1, day 3, day 6 post-washing and normalized with TurboGFPdes median of unstimulated cells (n=3). (C) Reversibility of the inducible promoter. Reversibility of pNR4A1(734-bp)-responsive TurboGFPdes expression was assessed by cultivating BJAB cells transduced with lentiviral vectors encoding the TurboGFPdes under the 734-bp inducible reporter or the SFFV constitutive reporter while alternating 8 h of stimulation (grey) and 80 h of rest (white) three times with F(ab′)2 IgM or PMA combined with ionomycin. For the PMA/ionomycin conditions, only one round of stimulation was performed because of important cell death. TurboGFPdes median expression was assessed before and after stimulation and normalized by TuroboGFPdes median unstimulated cells (n=3).

FIG. 4 represents the design and validation process of a sensor component. (A) BJAB cells (IgM+, IgG−) were transduced with lentiviral vectors encoding a sensor component, a membrane anchored BCR, recognizing either OVA (FAM0-OVA) or the HbS glycoprotein of HBV (FAM0-ADRI) 5 days before staining of IgG by western blot (WB). (B) Specific recognition of OVA by membrane-anchored BCR directed against OVA. BJAB cells were transduced with lentiviral vectors encoding membrane-anchored BCR targeting either OVA or HbS. Five days after transduction, these cells were incubated during 24 h with OVA-coated fluorescent beads. Binding fluorescent beads was assessed by flow cytometry. (C) Signaling and cell activation after SPAGs-OVA binding. BJAB cells were transduced with tagged or not membrane BCRs recognizing HbS or OVA were incubated 24 h with SPAGs-OVA before staining with an anti-anti-CD86 or an anti-HLA-DR antibody. One representative overlay is presented along with the median fluorescence intensity was quantified from three experiments for both markers (ANOVA1 and multiple comparison with Tukey correction, n=4).

FIG. 5 shows the antigen-specific activation of the nucleic acid system. BJAB cells were transduced with lentiviral vectors encoding SFFV-TurboGFPdes or pNR4A1(734)-TurboGFPdes one week before transduction with lentiviral vectors encoding membrane-anchored BCR directed against OVA (FAM0-OVA) or against HbS (FAM0-ADRI). Double transduced cells were then stimulated either with F(ab′)2 directed against IgM or IgG, or PLA/ionomycin as a control (A) or with OVA/Spike RBD coated beads with or without CD40L (B) for 24 h before detection of TurboGFPdes fluorescence by flow cytometry. ANOVA 2 post hoc comparison with Tukey correction (n=5/6) ***: Pvalue <0.001.

FIG. 6 represents the inducibility of the promoter in T cells. Jurkat cells were transduced with the 734 bp pNR4A1 reporter construct before stimulation with anti CD3 and anti CD28 (1 μg/ml) antibodies or beads (TransAct) or PMA (15 ng/ml) combined with ionomycin (1 M) during 24 h. (A) GFP induction of the reporter constructs following 24 h of TCR stimulation. (B) Median GFP expression of GFP+ cells after stimulation was normalized by the non-stimulated condition. Data are representative of four independent experiments (n=4, ANOVA 2 and multiple t-tests with Sidak Bonferroni correction (alpha=0.05)).

FIG. 7 shows the all-in-one vectors encoding a self-amplifying circuit. (A) illustrates the structure of the self-amplifying vector. The inducible pNR4A1-734 bp fragments drives both the TurboGFPdes and the FAM0-OVA sensor transgenes with a T2A sequence in between. (B) and (E) show the self-amplifying vector induction of effector expression in transduced BJAB cells after 24 h (B) or 48 h (E) stimulation with F(ab′)2 IgM or F(ab′)2 IgG. (C) and (F) illustrate the self-amplifying vector induction of sensor expression in transduced BJAB cells after 24 h (C) or 48 h (F) stimulation with F(ab′)2 IgM or F(ab′)2 IgG. The fold change of the mRNAs encoding the sensor was assessed by RT-qPCR after stimulation. (D) and (G) show the self-amplifying vector induction of effector expression in transduced BJAB cells stimulated with OVA/Spike RBD coated Beads with or without CD40L for 24 h (D) or 48 h (G) before detection of TurboGFPdes fluorescence by flow cytometry. The median expression of TurboGFPdes was assessed by flow cytometry normalized by the median of unstimulated cells. ANOVA 2 post hoc comparison with Tukey correction (n=5/6 for panels B, D, E, G and n=3 for panels C, F). Error bars represent SEM.

FIG. 8 demonstrates the higher basal level of TurboGFPdes signal in B cells transduced with the self-amplifying vector. (A) illustrates the leakage of TurboGFP expression without stimulation. The MFI of the TurboGFPdes was assessed in BJAB cells transduced with lentiviral vectors encoding the pNR4A1(734)-TurboGFPdes with or without the T2A-FAM0-OVA sequence after. The MFI was normalized by the vector copy number of transduced cells (n=3). (B) shows the TurboGFPdes half-life alone or followed by 16 amino acids of the T2A motif. Cycloheximide was added to transduced cells and the TurboGFPdes MFI was assessed every 40 minutes during 360 minutes. The half-life was then computed as the time to reach an MFI equal to the 0.5× of the initial MFI (n=3).

FIG. 9 shows the kinetics of extinction of the self-amplifying vector. BJAB cells were transduced with lentiviral vectors encoding the TurboGFPdes, alone or fused to the T2A-FAM0-OVA sequence, after the 734-bp inducible reporter. Transduced cells were stimulated 24 h with F(ab′)2 IgM (A) or F(ab′)2 IgG (B) before 3 washes and subsequent culturing. TurboGFPdes median expression was assessed at day 1, 3, 6 and 10 post-washing and normalized by TurboGFPdes median of unstimulated cells (n=3)

### DETAILED DESCRIPTION

### Definitions

The present invention concerns a nucleic acid system comprising:
(i) a sensor component comprising a sequence encoding
(a) at least one extracellular ligand recognition domain;
(b) a transmembrane domain of a B-cell receptor (BCR) or of a T-cell receptor (TCR); and
(c) optionally a signaling domain that controls cell activation of a B-cell receptor or of a T-cell receptor; and
(ii) a transducer/effector component comprising a sequence encoding
(d) the pNR4A1 promoter consisting of SEQ ID NO: 1, or a functional fragment thereof; and
(e) at least one effector protein of interest.

As shown in the Examples section, the inventors have demonstrated the efficacy of the nucleic acid system, in particular in a tumor model. The efficacy of this system may be applied to many other pathologies, such as auto immune disorders, transplantation rejection, allergies, neurological disorders, and infectious diseases.

Indeed, the main advantage of the nucleic acid system of the invention is its full programmability in terms of recognized signal and output functions, which can be adapted to the targeted disease, towards the same goal of continuous sensing of disease-specific biomarkers and triggering of physiological expression of therapeutic molecules in vivo in response. As such, the present system holds great promises for the long-term treatment of many diseases.

The inventors were also able to demonstrate that a lentivirus vector including a self-regulated construct comprising a nucleic acid system of the invention allowed specific expression of the effector protein upon sensor stimulation. As further explained below, such all-in-one LVs may increase the number of cells co-expressing all circuit components, thus reducing variability (i.e., avoiding cells partially modified with not all circuit components).

Unless otherwise defined herein, units, prefixes, and symbols are denoted in their Système International des Unités (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic characteristic(s) of the disclosure. It is understood that the different embodiments of the disclosure using the term “comprising” or equivalent cover the embodiments where this term is replaced with “comprising only”, “consisting of” or “consisting essentially of”.

It is understood that wherever aspects are described herein with the language “comprising,” otherwise analogous aspects described in terms of “consisting of” and/or “consisting essentially of” are also provided.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Within the disclosure, the terms “significantly” or “substantially” used to qualify a difference or a change, for example ‘significantly different of’ or “substantially different from”, with respect to a feature or a parameter intends to mean that the observe change or difference is noticeable and/or it has a statistic meaning. Conversely, the terms significantly” or “substantially” used to qualify a similitude or an identity, for example “not significantly different from” or “substantially identical to”, with respect to a feature or a parameter intends to mean that any observed change or difference is such that the nature and function of the concerned parameter or feature is not materially affected.

In the present text, the term “nucleic acid” refers to polynucleotides such as deoxyribonucleic acid (DNA), and, where appropriate, ribonucleic acid (RNA). Preferably, the nucleic acid is DNA. The term should also be understood to include, as equivalents, analogs of either RNA or DNA made from nucleotide analogs.

The term “isolated nucleic acid” as used herein, refers to a nucleic acid that is (i) free of sequences that normally flank one or both sides of the nucleic acid in a genome, (ii) incorporated into a vector or into the genomic DNA of an organism such that the resulting molecule is not identical to any naturally occurring vector or genomic DNA, or (iii) a cDNA, a genomic nucleic acid fragment, a fragment produced by polymerase chain reaction (PCR) or a restriction fragment. In addition, an isolated nucleic acid can include an engineered nucleic acid such as a recombinant DNA molecule that is part of a hybrid or fusion nucleic acid.

The nucleic acid can comprise coding and/or non-coding sequences. Coding nucleic acids have nucleotide sequences that are transcribed into RNA molecules that can be translated to create polypeptides. Non-coding nucleic acids, typically, are transcribed into RNAs that cannot be translated.

All the nucleic acid sequences defined in the present application may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the mRNA, or to avoid cryptic splicing sites as described in Fallot et al. (2009) Nucleic Acids Res. 37:e134 or Resse et al. (1997) J. Comput. Biol. 4:31 1-323. Codon optimization tools, algorithms and services are known in the art, non-limiting examples including services from GeneArt (Life Technologies), DNA2.0 (Menlo Park Calif.) and/or proprietary methods.

By “a sequence at least x % identical to a reference sequence”, it is intended that the sequence differs from the reference sequence by up to 100-x amino acid, respectively nucleotide, alterations per each 100 amino acids, respectively nucleotides, of the reference sequence. In particular, a sequence having at least 90% sequence identity with another sequence includes sequences having at least 91% sequence identity, at least 92% sequence identity, at least 93% sequence identity, at least 94% sequence identity, at least 95% sequence identity, at least 96% sequence identity, at least 97% sequence identity, at least 98% sequence identity and at least 99% sequence identity with another sequence.

The alignment and the determination of the percentage of identity may be carried out manually or automatically using for instance the Needle program which is based on the Needleman and Wunsch algorithm, described in Needleman and Wunsch (1970) J. Mol Biol. 48:443-453, with for example the following parameters for polypeptide sequence comparison: comparison matrix: BLOSUM62, gap open penalty: 10 and gap extend penalty: 0.5, end gap penalty: false, end gap open penalty=10, end gap extend penalty=0.5; and the following parameters for polynucleotide sequence comparison: comparison matrix: DNAFULL; gap open penalty=10, gap extend penalty=0.5, end gap penalty: false, end gap open penalty=10, end gap extend penalty=0.5.

In the context of the invention, the term “immunoglobulin” relates to proteins of the immunoglobulin superfamily (which also includes T-cell receptors), preferably to antigen receptors such as antibodies or the B cell receptor (BCR). The immunoglobulins are characterized by a structural domain, i.e., the immunoglobulin domain, having a characteristic immunoglobulin (Ig) fold (one or more). The term encompasses membrane-anchored immunoglobulins as well as secretory immunoglobulins. Membrane-anchored or membrane-bound immunoglobulins are also termed surface immunoglobulins or membrane immunoglobulins, which are generally part of the BCR.

Immunoglobulins generally comprise several chains, typically two identical heavy chains and two identical light chains which are linked via disulfide bonds. These chains are primarily composed of immunoglobulin domains, including the VL domain (light chain variable domain), the CL domain (light chain constant domain), the VH domain (heavy chain variable domain) and the CH domains (heavy chain constant domains) CH1, optionally a hinge region, CH2, CH3, and optionally CH4.

There are five main heavy chain classes (or isotypes) which determine the functional activity of an antibody molecule: mu (μ) for IgM, delta (δ) for IgD, gamma (γ) for IgG, alpha (a) for IgA and epsilon (F) for IgE. In the context of the invention, the immunoglobulin may be an IgM, IgD, IgG, IgA or IgE. Preferably, the immunoglobulin is an IgG. As well-known from the skilled person, the IgG isotype encompasses four subclasses: the subclasses lgG1, lgG2, lgG3 and lgG4. In the context of the invention, the immunoglobulin may be of any IgG subclass. Preferably, the immunoglobulin is an lgG1. As opposed to the heavy chains of secretory immunoglobulins, the heavy chains of membrane-anchored immunoglobulins comprise a transmembrane domain and a short cytoplasmic domain at their carboxy-terminus.

In mammals, there are two types of light chain, lambda (λ) and kappa (κ). Both types of light chains may associate indifferently with any class of heavy chain.

The immunoglobulin chains comprise a variable region and a constant region. The constant region is essentially conserved within the different isotypes of the immunoglobulins, wherein the variable part is highly divers.

The variable regions of both light (VL) and heavy (VH) chains determine binding recognition and specificity to the antigen. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The constant region domains of the light (CL) and heavy (CH) chains confer important biological properties such as antibody chain association, secretion, transplacental mobility, complement binding, and binding to Fc receptors (FcR).

As used herein, the term “constant regions of an immunoglobulin heavy chain” preferably refers to the regions of the immunoglobulin heavy chain composed of the CH1, optionally a hinge region, CH2, CH3, and optionally the CH4 domain, preferably comprising one or more, preferably all, potential linker and/or hinge regions. It is particularly preferred that the constant region of an immunoglobulin heavy chain comprises one or more cysteine residues which are capable of mediating the association with another constant region of an immunoglobulin heavy chain by disulfide-bonding.

As used herein, the term “B cell receptor” or “BCR” refers to the antigen receptor at the plasma membrane of B cells. The B cell receptor is generally composed of a membrane-anchored antibody, as defined above, associated with lg-a and lg-β heterodimers which are capable of signal transduction. Such a B cell receptor is described for example in Janeway et al. (Immunobiology: The Immune System in Health and Disease. 5th edition. Janeway C A Jr, Travers P, Walport M, et al. New York: Garland Science; 2001). The loci encoding these genes are: (i) for IgH: Gene ID: 3492, (ii) for IgKappa: Gene ID: 50802 and (iii) for IgLambda: Gene ID: 3535.

As used herein, the term “T cell receptor” or “TCR” refers to the antigen receptor at the plasma membrane of T cells which is responsible for recognizing fragments of antigen as peptides bound to major histocompatibility complex (MHC) molecules. It is generally composed of an alpha chain and a beta chain, comprising Ig domains. When the TCR engages with its specific antigenic peptide presented on MHC (peptide/MHC) by antigen presenting cells (MHC-II for CD4+ T cells) or other cells (MHC-I for CD8+ cells) if the costimulatory signal is provided through CD80/86 and CD28 interaction as well as the differentiation signal (cytokine secretion by the presenting cells), the T lymphocyte is activated. Depending on the cytokinic signal and on the T cell subtype (CD4 or CD8), the output functions can range from helping function to activate other immune cells to direct cytotoxicity against target cells. Such a B cell receptors are described for example in Janeway et al. (Immunobiology: The Immune System in Health and Disease. 5th edition. Janeway C A Jr, Travers P, Walport M, et al. New York: Garland Science; 2001).

As used herein, the term “chimeric immune receptor” (CIR) encompasses tumor- or virus-specific ligands or antibodies fused to the signaling domains of either the T cell receptor or Fc receptor of B cells. CIRs comprise an extracellular antigen binding domain, derived from an antigen-specific antibody or ligand, coupled to an intracellular signaling domain derived either from the CD3ζ chain of the T cell receptor complex, the Fc receptor (FcR) 7 chain tumor or more distal components of the T cell signal transduction pathway such as the syk molecule. Such a chimeric immune receptors are described for example in (i) Parvathaneni, Kalpana, et David W. Scott. «Engineered FVIII-Expressing Cytotoxic T Cells Target and Kill FVIII-Specific B Cells in Vitro and in Vivo». Blood Advances 2, no 18 (25 Sep. 2018): 2332-40. https://doi.org/10.1182/bloodadvances.2018018556, (ii) Sicard, Antoine, Megan K. Levings, et David W. Scott. «Engineering Therapeutic T Cells to Suppress Alloimmune Responses Using TCR s, CAR s, or BAR s». American Journal of Transplantation 18, no 6 (June 2018): 1305-11. https://doi.org/10.1111/ajt.14747, and in (iii) Ellebrecht, Christoph T., Vijay G. Bhoj, Arben Nace, Eun Jung Choi, Xuming Mao, Michael Jeffrey Cho, Giovanni Di Zenzo, et al. «Reengineering Chimeric Antigen Receptor T Cells for Targeted Therapy of Autoimmune Disease». Science 353, no 6295 (8 Jul. 2016): 179-84. https://doi.org/10.1126/science.aaf6756.

CIRs include CAR-T cells, CAR-NK cells, B-cell antibody receptors (BAR) and chimeric autoantibody receptor T (CAAR-T) cells.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell receptors signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. Chimeric antigen receptors are described notably in Feins, Steven, Weimin Kong, Erik F. Williams, Michael C. Milone, et Joseph A. Fraietta. «An Introduction to Chimeric Antigen Receptor (CAR) T-cell Immunotherapy for Human Cancer». American Journal of Hematology 94, no S1 (mai 2019): S3-9. https://doi.org/10.1002/ajh.25418.

As used herein, the term “CAR-NK cell” defines a CAR-engineered natural killer (CAR-NK) cell. Natural killer (NK) cells, due to their efficient recognition and lysis of malignant cells, are considered as specialized immune cells that can be genetically modified to obtain capable effector cells for adoptive cellular treatment of cancer patients. However, there are mechanisms by which the tumor escapes from the immune surveillance and inhibit the function of NK cells such as tumor microenvironment and immunosuppressive factors that prevent the expression of activating receptors and the interactions NK cells with other cells, also antigen escape ways can trigger inhibitory NK cell receptors and inhibit activating NK cell receptors. The CAR-NK technology consists in Chimeric Antigen Receptors equipped Natural Killer cells with the ability to recognize, target and kill specific cells. It is able to provide the results of CAR T-cell therapy without the high toxicity and risk of graft-versus-host disease CAR NK cells are described notably in (i) Schmidt, Paula, Martin J. Raftery, et Gabriele Pecher. «Engineering NK Cells for CAR Therapy—Recent Advances in Gene Transfer Methodology». Frontiers in Immunology 11 (7 Jan. 2021): 611163. https://doi.org/10.3389/fimmu.2020.611163, and in (ii) Yilmaz, Ahmet, Hanwei Cui, Michael A. Caligiuri, et Jianhua Yu. «Chimeric Antigen Receptor-Engineered Natural Killer Cells for Cancer Immunotherapy». Journal of Hematology & Oncology 13, no 1 (7 Dec. 2020): 168. https://doi.org/10.1186/s13045-020-00998-9.

New CIR approaches have been developed to inhibit or eliminate antibody-producing B cells that are undesirable in autoimmune diseases such as pemphigus vulgaris (PV) and hemophilia A. These BAR-T (B-cell antibody receptor T cells) or CAAR-T (chimeric auto-antibody receptor T cells) cells express a transmembrane and intracellular domain similar to that of CAR-T cells but have a specific antigen on their surface in place of the classical scFv fragment. These BAR/CAAR cells trap autoreactive LB by inducing recognition between the antigen exposed on the surface of the LT and the BCR. The binding between these 2 molecules activates the CIR, thus releasing the cytotoxic potential of the modified T cells, to specifically eliminate antigen-specified B cells.

As used herein, “chimeric autoantibody receptor T (CAAR-T) cells” or “B cell antibody receptor” (BAR) are the modified form of CAR-T cells which identify cells secreting antibodies like autoreactive B cells. The construction of CAAR-T cells consists of a specific antigen, a transmembrane domain, and an intracellular signaling domain with or without a co-stimulatory domain. CAAR-T cells recognize and bind to the target autoantibodies expressed on autoreactive cells via the specific antigen, and subsequently, destroy them. The skilled artisan may refer to (i) Parvathaneni, Kalpana, et David W. Scott. «Engineered FVIII-Expressing Cytotoxic T Cells Target and Kill FVIII-Specific B Cells in Vitro and in Vivo». Blood Advances 2, no 18 (25 Sep. 2018): 2332-40. https://doi.org/10.1182/bloodadvances.2018018556, (ii) Sicard, Antoine, Megan K. Levings, et David W. Scott. «Engineering Therapeutic T Cells to Suppress Alloimmune Responses Using TCR s, CAR s, or BAR s». American Journal of Transplantation 18, no 6 (June 2018): 1305-11. https://doi.org/10.1111/ajt.14747, and to (iii) Ellebrecht, Christoph T., Vijay G. Bhoj, Arben Nace, Eun Jung Choi, Xuming Mao, Michael Jeffrey Cho, Giovanni Di Zenzo, et al. «Reengineering Chimeric Antigen Receptor T Cells for Targeted Therapy of Autoimmune Disease». Science 353, no 6295 (8 Jul. 2016): 179-84. https://doi.org/10.1126/science.aaf6756.

By “antibody of interest” is meant herein an immunoglobulin, as defined above, comprising a light chain variable domain and a heavy chain variable domain which determine its antigen specificity, and a light chain constant domain and heavy chain constant domains, and which is intended to be produced by a cell. Preferably, the amino acid sequence of the antibody of interest and/or the coding sequence of the antibody of interest and/or the sequence of the gene encoding the antibody of interest is known or can be determined by the skilled person.

The antibody of interest is preferably a monoclonal antibody. The antibody of interest may also be a chimeric antibody.

A “chimeric” antibody refers to an antibody made up of components from at least two different sources. In certain embodiments, a chimeric antibody comprises a portion of an antibody derived from a first species fused to another molecule, e.g., a portion of an antibody derived from a second species. In certain such embodiments, a chimeric antibody comprises a portion of an antibody derived from a non-human animal fused to a portion of an antibody derived from a human. In certain such embodiments, a chimeric antibody comprises all or a portion of a variable region of an antibody derived from a non-human animal fused to a constant region of an antibody derived from a human.

In particular, the antibody of interest may be a humanized antibody. A “humanized” antibody refers to a non-human antibody that has been modified so that it more closely matches (in amino acid sequence) a human antibody. In certain embodiments, amino acid residues outside of the antigen binding residues of the variable region of the non-human antibody are modified. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In certain embodiments, a humanized antibody is constructed by replacing all or a portion of a CDR of a human antibody with all or a portion of a CDR from another antibody, such as a non-human antibody, having the desired antigen binding specificity. In certain embodiments, a humanized antibody comprises variable regions in which all or substantially all of the CDRs correspond to CDRs of a non-human antibody and all or substantially all of the framework regions (FRs) correspond to FRs of a human antibody. In certain such embodiments, a humanized antibody further comprises a constant region (Fc) of a human antibody. Furthermore, humanized antibodies may comprise residues that are not found in the recipient antibody or in the donor antibody.

The antibody of interest may further be a human antibody.

The term “human antibody” refers to a monoclonal antibody that contains human antibody sequences and does not contain antibody sequences from a non-human animal. In certain embodiments, a human antibody may contain synthetic sequences not found in native antibodies. The term is not limited by the manner in which the antibodies are made. For example, in various embodiments, a human antibody may be made in a transgenic mouse, by phage display, by human B-lymphocytes, or by recombinant methods.

As used herein, the term “B cell” refers to a B lymphocyte. B cell precursors reside in the bone marrow where immature B cells are produced. Very briefly, B cell development occurs through several stages, each stage representing a change in the genome content at the antibody loci. In the genomic heavy chain variable region, there are three segments, V, D, and J, which recombine randomly, in a process called VDJ rearrangement to produce a unique variable region in the immunoglobulin of each B cell. Similar rearrangements occur for the light chain variable region except that there are only two segments involved, V and J. After complete rearrangement, the B cell reaches the lgM<+>immature stage in the bone marrow. These immature B cells present a membrane-anchored IgM, i.e., BCR, on their surface and migrate to the spleen, where they are called transitional B cells. Some of these cells differentiate into mature B lymphocytes. Mature B cells expressing the BCR on their surface circulate the blood and lymphatic system performing the role of immune surveillance. They do not produce secretory immunoglobulins until they become fully activated. Each B cell has a unique receptor protein that will bind to one particular antigen. Once a B cell encounters its antigen and receives an additional signal from a T helper cell, it can further differentiate into either a plasma B cell expressing and secreting secretory immunoglobulins or a memory B cell.

As used herein, the term “T cell” refers to a T lymphocyte. The T lymphocytes are regulators or effectors of adaptive function, serving as primary effectors for cell-mediated immunity. They originate in the bone marrow and mature in the thymus. In the thymus, T cells multiply and differentiate into helper, regulatory, or cytotoxic T cells or become memory T cells. They are then sent to peripheral tissues or circulate in the blood or lymphatic system. Antigenic specificity is dictated by means of the TCR heterodimer receptor, derived from recombination of gene segments. CD4+T-helper lymphocyte cells recognize exogenous antigen presented in the context of MHC class II molecules. Different subclasses of T-helper cells secrete unique subsets of cytokines that assist in functional activity. These cells can also directly interact with other immune cells to provide the ‘help signal’ required to activate them. CD8+T lymphocytes, also called cytotoxic T cells (CTLs), recognize endogenous antigen presented in the context of MHC class I molecules. CTLs kill target cells directly by inducing apoptosis via released preformed proteins.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein. Every numerical range given throughout the specification will also include the lowest and highest values provided for the range.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

All publications and other references mentioned herein are incorporated by reference in their entirety. Although a number of documents are cited herein, this citation does not constitute an admission that any of these documents forms part of the common general knowledge in the art.

### Nucleic Acid System

A nucleic acid system according to the invention comprises
(i) a sensor component comprising a sequence encoding
(a) at least one extracellular ligand recognition domain;
(b) a transmembrane domain of a B-cell receptor (BCR) or of a T-cell receptor (TCR); and
(c) optionally a signaling domain that controls cell activation of a B-cell receptor or of a T-cell receptor; and
(ii) a transducer/effector component comprising a sequence encoding
(d) the pNR4A1 promoter consisting of SEQ ID NO: 1, or a functional fragment thereof; and
(e) at least one effector protein of interest.

### Sensor Component

The nucleic acid system of the invention comprises a first sensor component.

As defined herein, a “sensor” is the part of the nucleic acid system encoding a protein comprising at least one extracellular ligand recognition domain, which will recognize a pathological ligand, such as a pathological antigen, the said ligand being in some embodiments a disease biomarker. Upon activation through binding to a targeted ligand, the said extracellular ligand recognition domain generates one or more signals that will in turn activate the effector component of the nucleic acid system. Thus, recognition of a targeted ligand by the extracellular recognition domain encoded by the sensor component will trigger a signaling cascade leading to an output response from the reprogrammed cell.

The sensor component comprises sequences encoding (a) at least one extracellular ligand recognition domain; (b) a transmembrane domain of a B cell receptor (BCR) or of a T cell receptor (TCR); and optionally (c) a signaling domain that controls cell activation of a B cell receptor or of a T cell receptor.

Together, these domains form a cell-surface receptor that targets a pathological ligand, such as a disease-specific antigen, and the said cell surface receptor being activated upon its binding to the said pathological ligand, e.g. the said disease-specific antigen.

### Extracellular Ligand Recognition Domain

As mentioned above, one of the many advantages of a nucleic acid system of the invention is its ability to be adapted to the prevention and/or treatment of many types of conditions and diseases. This is in part possible because of the very high adaptability regarding the appropriate type of extracellular ligand recognition domain that may be selected for targeting a disease-specific pathological ligand.

In a particular embodiment, the extracellular ligand recognition domain comprises at least one ligand binding fragment. In particular, the ligand binding fragment is an antigen-binding domain.

In the context of the invention, the term “antigen-binding domain” refers to any peptide, polypeptide, scaffold-type molecule, peptide display molecule or polypeptide-containing construct that is capable of specifically binding a particular antigen of interest.

Antigen-binding domains include for example antigen-binding portions of antibodies, single-chain antibodies, single domain antibodies (e.g., VHH antibodies from camelid animals), peptides that specifically interact with a particular antigen (e.g. peptibodies), receptor molecules that specifically interact with a particular antigen, proteins comprising a ligand-binding portion of a receptor that specifically binds a particular antigen, antigen-binding scaffolds (e.g. DARPins, HEAT repeat proteins, ARM repeat proteins, tetratricopeptide repeat proteins and other scaffolds based on naturally occurring repeat proteins), and aptamers or portions thereof.

The antigen-binding domain may be formed by a single peptide or protein AbD, or by the combination of two antigen-binding domain subunits, AbD1 and AbD2, the combination of these two subunits enabling the specific interaction of the global antigen-binding domain with an antigen of interest.

In a particular embodiment, the antigen-binding domain of the invention may be selected from antigen-binding domains of antibodies such as Fab fragments, Fab′ fragments, F(ab′)2 fragments; Fd fragments, single domain antibodies (sdAb), complementary determining regions (CDR), Fv fragments, single chain FVs (scFV), dsFvs, and sc(Fv)2: from antigen-binding domains of antibody mimetics such as affibodies, affilins, affitins, adnectins, atrimers, evasins, DARPins, anticalins, avimers, fynomers, and versabodies; from aptamers, and mixtures thereof.

Alternatively, proteins can be directly coupled to the transmembrane domain and the recognition of this protein by a receptor expressed specifically on pathological cells will trigger the activation of the circuit.

Antigen-binding domains of antibodies refer to one or more fragments of an intact antibody that retain the ability to specifically bind to a given antigen. Antigen biding functions of an antibody can be performed by fragments of an intact antibody. Examples of biding fragments encompassed within the term antigen biding fragment of an antibody include a Fab fragment, a monovalent fragment consisting of the VL, VH, CL and CH1 domains; a Fab′ fragment, a monovalent fragment consisting of the VL, VH, CL, CH1 domains and hinge region; a F(ab′)2 fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen biding fragments can be incorporated into single chain molecules comprising a pair of tandem Fv segments (VH-CH1-VH-CH1) Which, together with complementary light chain polypeptides, form a pair of antigen binding regions (Zapata et al., 1995 Protein Eng. 8(10); 1057-1062 and U.S. Pat. No. 5,641,870).

The term “antibody mimetic” is intended to refer to molecules capable of mimicking an antibody's ability to bind an antigen, but which are not limited to native antibody structures. Examples of such antibody mimetics include, but are not limited to, Adnectins, Affibodies, DARPins, Anticalins, Avimers, and versabodies, all of which employ binding structures that, while they mimic traditional antibody binding, are generated from and function via distinct mechanisms. Antigen biding fragments of antibodies can be grafted into scaffolds based on polypeptides such as Fibronectin type III (Fn3) (see U.S. Pat. No. 6,703,199, which describes fibronectin polypeptide monobodies). An affibody is well known in the art and refers to affinity proteins based on a 58 amino acid residue protein domain, derived from one of the IgG binding domains of staphylococcal protein A. DARPins (Designed Ankyrin Repeat Proteins) are well known in the art and refer to an antibody mimetic DRP (designed repeat protein) technology developed to exploit the binding abilities of non-antibody proteins. Anticalins are well known in the art and refer to another antibody mimetic technology, wherein the binding specificity is derived from lipocalins. Anticalins may also be formatted as dual targeting protein, called Duocalins. Avimers are well known in the art and refer to another antibody mimetic technology, Avimers are derived from natural A-domain containing protein. Versabodies are well known in the art and refer to another antibody mimetic technology, they are small proteins of 3-5 kDa with >15% cysteines, which form a high disulfide density scaffold, replacing the hydrophobic core the typical proteins have. Such antibody mimetic can be comprised in a scaffold. The term “scaffold” refers to a polypeptide platform for the engineering of new products with tailored functions and characteristics.

The fibronectin scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprise the entire antigen recognition unit in camel and llama IgG. Because of this structure, the non-immunoglobulin antibody mimics antigen binding properties that are similar in nature and affinity to those of antibodies. These scaffolds can be used in a loop randomisation and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The Ankyrin technology is based on using proteins with Ankyrin derived repeat modules as scaffolds for bearing variable regions which can be used for binding to different targets. The Ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as LRP-1. These domains are used by nature for protein-protein interactions and in human over 250 proteins are structurally based on “A-domains” monomers (2-10) linked via amino acids linkers. Avimers can be created that can bind to the target antigen using the methodology described in, for example, U.S. patent Application publication Nos. 20040175756; 20050053973; 20050048512; and 20060008844.

Affibody affinity ligands are small, simple proteins composed of a three-helix bundle based on the scaffold of one of the IgG-binding domains of protein A. protein A is a surface protein form the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acids residues, being just marginally bigger than a single immunoglobulin domain. The set of four loops, which makes up the binding pocket, shows pronounced structural plasticity and tolerates a variety of side chains. The binding site can can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of “ubiquitin-like” proteins are described in WO2004106368.

Versabodies are highly soluble and can be formulated to high concentrations. Versabodies are exceptionally heat stable and offer extended shelf-life. Additional information regarding Versabodies can be found in US 2007/0191272, which is hereby incorporated by reference in its entirety.

Aptamers are stable DNA or RNA ligands that bind with high affinity and specificity to target antigens such as small molecules, peptides, proteins, cells, and tissues. These molecules present many advantages: they have prolonged shelf life, present low batch to batch variation, show low or no immunogenicity and allow freedom to incorporate chemical modification for enhanced stability and targeting capacity. As such, they find their application in various fields from therapy, drug delivery, diagnosis, and functional genomics to bio-sensing. Aptamers are generally developed in vitro by a very defined iterative procedure known as Systematic Evolution of Ligands by Exponential enrichment (SELEX) (Wang et al., 2019, Biotech. Adv., vol 37, issue 1, 28-50). RNA aptamer technologies are described in U.S. Pat. Nos. 5,789,157; 5,864,026; 5,712,375; 5,763,566; 6,013,443; 6,376,474; 6,613,526; 6,114,120; 6,261,774; and 6,387,620.

The ligand-binding domain is capable of binding to a ligand of interest.

Upon binding with the ligand-binding domain, the ligand of interest will activate the sensor component of the nucleic acid system.

According to the condition or disease to be treated, a person skilled in the art will know to target a given ligand of interest, and as such select an appropriate ligand-binding domain.

The ligand of interest may be inorganic, such as ions. The presence of these ligands, in particular in an abnormal amount, may reflect a dys-regulation of the microenvironment, which may appear in an individual suffering from a pathological condition or disease, such as cancer.

The ligand of interest may also be organic, such as peptides or proteins. Such ligands are often present on the outer wall of target cells and organisms or in the form of free molecules. They are recognized and targeted by the immune system of individuals. Protein ligands may for example be present on tumor cells, on “self” cells in the case of autoimmune diseases, on cells from transplanted tissue, on viruses, on bacteria and on other infectious microorganisms, and allergens.

In a particular embodiment, the ligand of interest in an antigen.

In a particular embodiment, the ligand of interest is selected from one or more tumor antigens, one or more self-antigens, one or more allo-antigens, one or more viral antigens, one or more bacterial antigens, one or more allergen antigens, or one or more markers of neurological disorders.

More generally, any molecule indicating a dysregulation of homeostasis and a pathological environment can be used as a ligand.

Many of these antigens have been identified in the past, during the study of specific conditions and diseases, and are well known in the art.

Tumor antigens include those expressed by tumor cells in the case of the tumors or cancers provided in the list further below in the specification. It can be tumor associated antigens or the perfect candidates will be tumor specific antigens, which are rarer.

Examples of tumor antigens include PSA, mesothelin, Human Epidermal Receptor 2, oncoviral proteins (from HPV, EBV viruses), carcinoembryonic antigens (CEA), oncofetal antigens, alpha-fetoprotein, CA-125, MUC-1, Epithelial tumor antigen (ETA), tyrosinase, Melanoma-associated antigens (MAGE), etc.

Viral antigens include antigens presented by the viruses provided in the list further below in the specification. Bacterial antigens include antigens presented by the bacteria provided in the list further below in the specification.

Self-antigens are antigens in the body of an individual, which are not normally available to the immune system. In regard to autoimmune diseases, they are those cellular proteins, peptides, enzyme complexes, ribonucleoprotein complexes, DNA, and post-translationally modified antigens against which autoantibodies are directed. Indeed, infective or physical tissue damage, particularly in a genetically susceptible individual, or a defect in the phagocytic removal of apoptotic cells, may terminate immunological tolerance to self-antigens, leading to autoimmune disease.

In some embodiments, the ligand of interest is Factor VIII, so as to prevent or treat hemophilia A. Hemophilia A is a disorder caused by mutations in the factor VIII (FVIII) gene (F8). Treatment with recombinant or plasma-derived FVIII replacement therapy is the conventional therapy but a major problem with this kind of treatment is that 20% to 30% of these patients produce neutralizing anti-FVIII antibodies (inhibitors) because they are not immunologically tolerant to this human protein. The goal is to render them tolerant to this protein so that their immune system stops recognizing it as foreign and stops degrading it.

### Transmembrane Domain of a B Cell Receptor or of a T Cell Receptor

As used herein, the term “transmembrane domain of a B cell receptor or a T-cell receptor” preferably refers to:
the transmembrane domain of the membrane-anchored immunoglobulin part of the B cell receptor, i.e., the transmembrane domain of the membrane-anchored immunoglobulin heavy chain, or to
the TCR transmembrane domain which links the extracellular variable-like region (VR) and constant-like region (CR) and the stalk segment allowing chain pairing by a disulfide bond of the T cell receptor to the short cytoplasmic tail. The transmembrane helices of both chains alpha and beta are unusual in containing positively charged residues with the hydrophobic transmembrane (respectively two and one).

These domains are essential in the anchoring of the sensor component to the cell.

Signaling Domain that Controls Cell Activation of a B Cell Receptor or of a T Cell Receptor

Interaction of the extracellular ligand recognition domain of the sensor component with an antigen of interest will lead to the activation of the signaling domains of the BCR, the TCR or the CIR, and thus to various signal transduction pathways within the host cell. BCR and TCR signaling pathways are crucial for proper B cell and T cell development, activation, proliferation, differentiation and consequently for humoral immune response.

As used herein, “signaling domain that controls cell activation of a B cell receptor” refers to disulfide-linked dimers of immunoglobulin (Ig)-α and Ig-β/γ subunits. These subunits, also known as CD79a and CD79b, are products of the mb-1(α) and B29 (β/γ) genes.

Engagement of the BCR by antigen induces membrane movement and aggregation of BCR components that lead to phosphorylation of ITAMs in the cytoplasmic tails of CD79a and CD79b. The latter is accomplished by the SRC family kinase LYN. The phosphorylated ITAMs recruit the spleen tyrosine kinase (SYK) to the receptor, where it becomes activated by phosphorylation of tyrosines, and propagates the signal activation to downstream signaling proteins. Activation of SYK plays a critical role in BCR signaling, initiating the formation of the BCR signalosome, the adaptor proteins, such as CD19 and B-cell linker (BLNK), and Bruton's tyrosine kinases (BTK), and signaling enzymes such as PLCγ2, PI3K, and Vav. Signals emanating from those signalosomes initiate and regulate downstream signaling systems including RAS/RAF/MEK/ERK pathway, which are significant for B cell fate decisions such as proliferation, survival, differentiation and cell death.

Therefore, in a particular embodiment, the signaling domain that controls cell activation of a B cell receptor comprises a CD79a and a CD79b.

In a particular embodiment, the signaling domain that controls cell activation of a B cell receptor comprises protein domains which bind with anchored immunoglobulin at the surface of the cell.

As used herein, “signaling domain that controls cell activation of a T cell receptor” refers to the signal transducing subunits γ, δ, ε and ζ (CD3 complex) of a T cell receptor, in combination or not with co-stimulatory domains (such as ICOS, 41BB, CD28).

The stimulus that drives T cell activation is a foreign antigen, in particular a peptide, bound to major histocompatibility complex (MHC)-encoded molecules presented on the surface of professional antigen-presenting cells (APC) such as dendritic cells (DC).

Therefore, in a particular embodiment, the signaling domain that controls cell activation of a T cell receptor comprises a CD3 complex comprising subunits γ, δ, ε and ζ of a T cell receptor. Binding of the MHC antigen complex on T cells leads to the phosphorylation of the ITAM motifs by Lck. ZAP-70 binds to the phosphorylated ζ-chain ITAMs and is phosphorylated and activated. Activated ZAP-70 then phosphorylates LAT and SLP-70, which are bound by GADS. The GADS:SLP-76:LAT recruits and activates PLC-γ through its phosphorylation by Itk. PLC-γ will then generate a calcium flux, and the indirect activation of the NFAT and NF-kB transcription factors.

The presence of a sequence encoding a signaling domain that controls cell activation of a B-cell receptor or of a T-cell receptor (c) is optional in the sensor component of the nucleic acid system of the invention.

Indeed, in certain embodiments, the cell, in particular the B cell or the T cell, that will be transformed with a nucleic acid system of the invention will already contain, at its surface, a signaling domain that controls cell activation of a B-cell receptor or of a T-cell receptor. In this case, the sensor component of the nucleic acid system of the invention may only comprise (a) a sequence encoding at least one extracellular ligand recognition domain and (b) a sequence encoding a transmembrane domain of a B-cell receptor (BCR) or of a T-cell receptor (TCR). This sensor component, when activated by a ligand via the extracellular ligand recognition domain, will consequently activate the signaling domain already present at the surface of the B cell or T cell, which will in turn activate the transducer/effector component. Similar mechanisms are at stake for chimeric receptors.

In such cases, the sensor component will not form a full receptor such as a B-cell receptor, a T-cell receptor, a chimeric immune receptor (CIR), such as a CAR-T cell, a CAR-NK cell, a B-cell antibody receptor (BAR) or a chimeric autoantibody receptor T (CAAR-T) cell. The sensor component anchored at the membrane will restrict the specificity of the target antigen and bring part of the elements of such a receptor, and use other elements, such as signalling domains and transcription factors, which are already inherently present at/in the surface of the cell.

In other embodiments, the sensor component of the nucleic acid system of the invention will comprise (a) a sequence encoding at least one extracellular ligand recognition domain, (b) a sequence encoding a transmembrane domain of a B-cell receptor (BCR) or of a T-cell receptor (TCR) and (c) a sequence encoding a signaling domain that controls cell activation of a B-cell receptor or of a T-cell receptor. This embodiment will be particularly applied if the cell host does not contain such a signaling domain at its surface.

In a particular embodiment, the sensor component of the nucleic acid system forms an immunoglobulin. In particular, the sensor component of the nucleic acid system forms an antigen receptor.

In a particular embodiment, the extracellular ligand recognition domain (a) the transmembrane domain (b) and optionally the signaling domain (c) form a B-cell receptor, a T-cell receptor, a chimeric immune receptor (CIR), such as a CAR-T cell, a CAR-NK cell, a B-cell antibody receptor (BAR) or a chimeric autoantibody receptor T (CAAR-T) cell, and in particular form a B-cell receptor.

In particular, when the sensor component forms a BCR, the sensor component preferably does not contain (c) a sequence encoding a signaling domain that controls cell activation of a B-cell receptor or of a T-cell receptor.

In particular, when the sensor component forms a TCR, the sensor component preferably does not contain (c) a sequence encoding a signaling domain that controls cell activation of a B-cell receptor or of a T-cell receptor.

In particular, when the sensor component forms a CIR, such as a CAR-T cell, a CAR-NK cell, a BAR or a CAAR-T cell, the sensor component preferably comprises (c) a sequence encoding a signaling domain that controls cell activation of a T-cell receptor.

Definitions for a B-cell receptor, a T-cell receptor and a chimeric immune receptor are provided above.

Thus, in some embodiments, the sensor component of the nucleic acid system disclosed herein encodes a protein comprising, from its N-terminal end to its C-terminal end, a signaling domain that controls cell activation of a B-cell receptor or of a T-cell receptor, a transmembrane domain of a B-cell receptor (BCR) or of a T-cell receptor (TCR) and at least one extracellular ligand recognition domain.

In some other embodiments, the sensor component of the nucleic acid system disclosed herein encodes a protein consisting of, from its N-terminal end to its C-terminal end, a signaling domain that controls cell activation of a B-cell receptor or of a T-cell receptor, a transmembrane domain of a B-cell receptor (BCR) or of a T-cell receptor (TCR) and at least one extracellular ligand recognition domain.

### Transducer/Effector Component

pNR4A1 Promoter

The effector component of the nucleic acid system of the invention comprises (d) a sequence encoding the pNR4A1 promoter, or a functional fragment thereof.

The NR4A1 gene, also known as the Nur77 gene, is an immediate early gene whose expression is rapidly upregulated by BCR or TCR signaling in murine cells and human thymocytes. It has been shown to be specifically induced in human T cells and B cells following respectively TCR and BCR stimulation.

As such, the pNR4A1 promoter is an inducible promoter which is activated upon TCR or BCR activation.

The pNRA41 sequence is known in the art. The isolated promoter sequence consists of SEQ ID NO: 1.

In a particular embodiment, the transducer/effector component of the nucleic acid system of the invention comprises a pNR4A1 promoter consisting of SEQ ID NO: 1.

In another embodiment, the transducer/effector component of the nucleic acid system of the invention comprises a functional fragment of the pNR4A1 promoter. As such, said functional fragment consists in a functional fragment of the sequence SEQ ID NO: 1.

As used herein, a “functional fragment” of the pNR4A1 promoter is a fragment which maintains the promoter activity of pNR4A1. As such, a functional fragment of pNR4A1 is capable of being activated upon TCR or BCR activation, in the same manner as the whole pNR4A1. It is also capable of being recognized by an RNA polymerase and the associated transcription factors and therefore initiating the transcription of the genes that are downstream from the fragment.

In a particular embodiment, the functional fragment of the pNR4A1 promoter has a length from 200 bp to 2210 bp, in particular from 500 bp to 2210 bp.

Fragments of from 200 bp to 2210 bp include fragments of the following lengths 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, and 2210.

Indeed, the inventors found that pNR4A1 fragments as small as 200 bp, and in particular as small as 500 bp, could be used as efficient promoters.

In a particular embodiment, the pNR4A1 promoter is selected from the fragments (functional fragments) having a nucleic acid sequence selected from the group consisting of SEQ ID NO:2, SEQ ID NO:3, SEQ ID NO:4, SEQ ID NO:5, SEQ ID NO: 22 and SEQ ID NO: 23, in particular from the group consisting of SEQ ID NO:2, SEQ ID NO:3, SEQ ID NO:4 and SEQ ID NO:5.

In a particular embodiment, the pNR4A1 promoter is selected from fragments having at least 90% sequence identity with SEQ ID NO:2, SEQ ID NO:3, SEQ ID NO: 4, SEQ ID NO: 5, SEQ ID NO: 22 or SEQ ID NO: 23, particularly with SEQ ID NO:2, SEQ ID NO:3, SEQ ID NO:4 and SEQ ID NO:5, in particular having at least 91%, at least 92%, at least 93%, at least 94%, at least 95%, at least 96%, at least 97%, at least 98% or at least 99% sequence identity with SEQ ID NO:2, SEQ ID NO:3, SEQ ID NO: 4, SEQ ID NO: 5, SEQ ID NO: 22 or SEQ ID NO: 23.

In a particular embodiment, the pNR4A1 promoter is selected from the fragments consisting of the sequences SEQ ID NO:2, SEQ ID NO:3, SEQ ID NO:4, SEQ ID NO:5, SEQ ID NO: 22 or SEQ ID NO: 23, in particular of the sequences SEQ ID NO:2, SEQ ID NO:3, SEQ ID NO:4 or SEQ ID NO:5.

In particular, the pNR4A1 promoter is the fragment having the sequence SEQ ID NO: 5.

In particular, the pNR4A1 promoter is the fragment having at least 90% sequence identity with SEQ ID NO:5, in particular having at least 91%, at least 92%, at least 93%, at least 94%, at least 95%, at least 96%, at least 97%, at least 98% or at least 99% sequence identity with SEQ ID NO:5.

In a particular embodiment, the pNR4A1 promoter is the fragment consisting of SEQ ID NO: 5.

### Sequence Encoding at Least One Effector Protein of Interest

Activation of the pNR4A1 promoter, or a functional fragment thereof, will lead to the transcription of at least one effector protein of interest.

In the context of the invention, the term “effector protein” refers to a protein that is involved in the regulation of a biological signaling pathway. Preferably, the effector protein is an immune effector protein or a cell death inducing effector protein.

By “immune effector protein” is meant herein a protein that is involved in the regulation of an immune system pathway. Examples of immune effector proteins include cytokines.

By “cell death inducing effector protein” is meant herein a protein that is involved in cell death signaling pathway, in particular in apoptosis signaling pathway.

The effector protein of interest may be formed by a single peptide or protein EfP, or by the combination of two effector protein subunits, EfP1 and EfP2, the combination of these two subunits forming an active effector protein EfP. By “combination” is meant herein any interaction linking two subunits defined above, such as a disulfide bond.

The effector protein of interest according to the invention will vary according to the disease to be treated or to the physiological state to be modified.

As such, when treating a tumor, the at least one effector molecule may be selected from pro-inflammatory molecules or cell death inducing molecules and mixtures thereof.

When treating an immune disease, the at least one effector molecule may be selected from tolerizing molecules or anti-inflammatory cytokines and mixtures thereof.

When avoiding a transplantation rejection, the at least one effector molecule may be selected from tolerizing/tolerogenic molecules and mixtures thereof.

When treating an allergy, the at least one effector molecule may be selected from anti histamine molecules and mixtures thereof.

When treating an infectious disease, the at least one effector molecule may be selected from anti-viral proteins and mixtures thereof.

When treating a neurological disorder, the at least one effector molecule may be selected from inhibitors of matrix metalloproteinases, neurotrophic factors such as nerve growth factors (to promoter neuronal growth), antibodies against B amyloid sheets, interference RNAs, modified dopamine, etc.

In a particular embodiment, the at least one effector protein of interest is an immunostimulatory protein or an immunosuppressant protein.

In particular, the at least one effector protein of interest is selected from pro-inflammatory cytokines, such as IL-18, gamma-interferon and TNF; anti-inflammatory cytokines, such as IL-10 and IL-4; co-stimulation molecules, such as CD80, CD86 and CD40; and inhibitor molecules, such as FasL and Fas.

However, any therapeutic molecule that can be encoded genetically can be implemented as the effector of the circuit.

In a particular embodiment, the (i) sensor component and the (ii) transducer/effector components of the nucleic acid system of the invention are both under the control of the pNR4A1 promoter consisting of SEQ ID NO: 1, or a functional fragment thereof.

In a particular embodiment, said pNR4A1 promoter consisting of SEQ ID NO: 1, or a functional fragment thereof is the only promoter present in the nucleic acid system. As such, according to a particular embodiment, the (i) sensor component and the (ii) transducer/effector components of the nucleic acid system of the invention are both under the control of the same pNR4A1 promoter consisting of SEQ ID NO: 1, or a functional fragment thereof.

When the (i) sensor component and the (ii) transducer/effector components of the nucleic acid system of the invention are both under the control of the same pNR4A1 promoter consisting of SEQ ID NO: 1, or a functional fragment thereof, the nucleic acid system further comprises an ORF separating sequence. Such a sequence may allow the multicistronic expression of the two components, i.e. the sensor and the transducer/effector. In particular, said ORF separating sequence is located between the (i) sensor component and the (ii) transducer/effector components.

Therefore, in a particular embodiment, the nucleic acid system further comprises (iii) an ORF separating sequence placed between the (i) sensor component and the (ii) transducer/effector component and said (i) sensor component and (ii) transducer/effector component are both under the control of the pNR4A1 promoter consisting of SEQ ID NO: 1, or a functional fragment thereof.

ORF separating sequences are nucleic acid sequences which are used to obtain more than one separate proteins from ORFs which are under the control of a single promotor. Examples of ORF separating sequences are known in the art. As such, the sensor and effector components may be placed within the same transcriptional unit using an ORF separating sequence between them.

Examples of ORF separating sequences include 2A self-cleaving peptides. 2A peptides consist of 18-22 aa-long peptides, which can induce ribosomal skipping during translation of a protein in a cell. This allows the generation of two proteins, i.e., the (a) sensor component and the (b) transducer/effector components, by causing the ribosome to fail at making a peptide bond. These sequences are described for instance in Liu et al. (Systematic comparison of 2A peptides for cloning multi-genes in a polycistronic vector. Sci Rep 7, 2193 (2017)).

Another ORF separating sequence to allow co-expression of two proteins which can be used is the internal ribosome entry site (IRES) (Maillot et al., Viral internal ribosomal entry sites: four classes for one goal, WIREs RNA 2018, 8:e1458)), such as IRES derived from the picornavirus.

ORF separating sequences may also include cleavage sequences which are recognized by intracellular proteases.

Another example of an ORF separating sequence is a linker sequence consisting of a 74 nucleotides (nt) between the first and second ORFs, such as the sensor component and the effector/transducer component, that has no ATG triplets and that provides an optimal distance between the stop codon of the first ORF and the start codon of the second ORF to allow reinitiation of translation (Kozak, M. 1987. Effects of intercistronic length on the efficiency of reinitiation by eucaryotic ribosomes. Mol. Cell. Biol. 7:3438-3445).

In a particular embodiment, the ORF separating sequence may be selected from a 2A self-cleaving peptide, such as a T2A, an IRES, a cleavage sequence recognized by intracellular proteases or a linker sequence. In particular, the ORF separating sequence is a 2A self-cleaving peptide.

In a particular embodiment, said ORF separating sequence is a T2A having a nucleic acid sequence of SEQ ID NO: 18.

Therefore, the sensor and effector components may be placed within the same transcriptional unit using a T2A sequence.

According to a particular embodiment, the invention relates to a nucleic acid system comprising:
(i) a sensor component comprising a sequence encoding
(a) at least one extracellular ligand recognition domain;
(b) a transmembrane domain of a B-cell receptor (BCR) or of a T-cell receptor (TCR); and
(c) optionally a signaling domain that controls cell activation of a B-cell receptor or of a T-cell receptor; and
(ii) a transducer/effector component comprising a sequence encoding
(d) the pNR4A1 promoter consisting of SEQ ID NO: 1, or a functional fragment thereof; and
(e) at least one effector protein of interest,
(iii) an ORF separating sequence placed between the (i) sensor component and the (ii) transducer/effector component,
wherein the (i) sensor component and the (ii) transducer/effector component of the nucleic acid system are both under the control of the pNR4A1 promoter consisting of SEQ ID NO: 1.

### Vectors

A further subject of the present invention relates to a vector comprising a nucleic acid system as described above.

Typically, the nucleic acid system of the invention is a DNA or RNA molecule, which may be included in any suitable vector, such as a linear DNA, a plasmid, cosmid, episome, artificial chromosome, phage or a viral vector.

As used herein, the terms “vector”, “cloning vector” and “expression vector” mean the vehicle by which a DNA or RNA sequence (e.g. a foreign gene) can be introduced into a cell, so as to transform the and promote expression (e.g. transcription and translation) of the introduced sequence.

Such vectors may comprise further regulatory elements, such as a promoter, enhancer, terminator and the like, to cause or direct expression of said nucleic acid system upon administration to a subject.

Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

In a particular embodiment, the vector further comprises at least one safety element which will allow the end-user to activate and disactivate the nucleic acid system of the invention at will. Safety elements will add another level of regulation for the system and will avoid ectopic expression of the system in particular in the absence of the antigen of interest. Safety elements which may be used according to the invention include inducible promoters, suicide genes, Boolean logic gates, etc.

In a particular embodiment, the vector as described herein further comprises at least one sequence encoding an inducible promoter and the effector protein of interest.

In a particular embodiment, the vector as described further comprises at least one sequence encoding a suicide gene.

As used herein, a “suicide gene” is a gene that will send an apoptosis death signal to the cell that expresses it, causing its destruction. Such a gene may be included in a vector according to the invention in order to avoid any serious adverse events by switching off the nucleic acid system.

Any expression vector for animal cell can be used, so long as a gene encoding the human antibody C region can be inserted and expressed.

Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

In a particular embodiment, the vector is a viral vector, in particular a retroviral vector.

The retroviral vector may be a vector plasmid including the nucleic acid system to be transferred to a target cell. The retroviral vector thus typically includes a retroviral backbone, containing cis-acting genetic sequences of a retrovirus necessary for the vector to infect the target cell, and the nucleic acid system according to the invention. The retroviral backbone thus typically includes the Long Terminal Repeats (LTRs) for the control of transcription and integration, the psi sequence necessary for encapsidation, and the Primer Binding Site (PBS) and polypurine track (PPT) sequences necessary for reverse transcription of the retroviral genome.

By “retrovirus” is meant a virus whose genome consists of a RNA molecule and that comprises a reverse-transcriptase, i.e. a member of the Retroviridae family. Retroviruses are divided into Oncovirus, Lentivirus and Spumavirus. Preferably, said retrovirus is an oncovirus, e.g. MLV, ALS, RSV or MPMV, a lentivirus, e.g. HIV-1, HIV-2, SIV, EIAV or CAEV, or a spumavirus such as HFV. Genomes of these retroviruses are readily available in databanks. More preferably, said retrovirus is a lentivirus, in particular HIV-1, HIV-2 or SIV.

As well-known from the skilled person, different types of transfer retroviral backbones can be used depending on the type of retroviral packaging system for which it is intended, namely the 1^{st} generation, 2^{nd} generation, 3^{rd} generation or 4^{rd} generation of retroviral packaging system.

As well-known from the skilled person, in the 1^{st} generation of retroviral packaging system, live viral particles are produced from one transfer retroviral vector which carries all the retrovirus genes, namely the genes encoding retroviral core proteins, enzymes and accessory factors together with the transgene, and from a separate plasmid bearing an envelope gene. The transgene is typically under control of a wild-type 5′-LTR.

In contrast, as well-known from the skilled person, in the 2^{nd} generation of retroviral packaging system, 5 of the 9 retrovirus genes are deleted, leaving only the gag/pol and tat/rev regions. The transgene is typically under control of a wild-type 5′-LTR. The gag/po 1/tat/rev regions are typically present on one separate plasmid.

As well-known from the skilled person, the 3^{rd} generation of retroviral packaging system contains only gag, pol and rev genes. The gag/pol and rev genes are typically present on two separate plasmids. The transgene is typically under control of a chimeric 5′-LTR to ensure transcription in the absence of tat. In this chimeric 5′-LTR, the U3 region is typically replaced by a constitutively active promoter/enhancer, such as RSV or CMV.

Finally, as well-known from the skilled person, in the 4^{rd} generation of retroviral packaging system, the gag and pol genes are further codon-optimized and present in two separate plasmids.

Preferably, the retroviral backbone contained in the retroviral vector as described herein is a third-generation or fourth generation retroviral backbone.

In a particularly preferred embodiment, the retroviral backbone contained in the retroviral vector is a self-inactivating retroviral backbone.

By “self-inactivating retroviral backbone” is meant herein a retroviral construct which has a deletion in the U3 element of the 3′-LTR of the construct, and which results, after replication, in a deletion also in the 5′-LTR promoter and enhancer and prevents the transcription from the cell-specific internal promoter, which may otherwise activate silent cellular oncogenes.

In a particularly preferred embodiment, the retroviral backbone contained in the retroviral vector as described herein is a 3^{rd} generation or 4^{rd} generation self-inactivating retroviral backbone, more particularly a 3^{rd} generation or 4^{rd} generation self-inactivating lentiviral backbone.

Thus, in a particular embodiment, the retroviral backbone contained in the retroviral vector comprises successively: (11) a modified 5′ LTR comprising a CMV enhancer substituted for the U3 region, (12) a psi and gag sequence, (13) a central polypurine tract (cPPT)/DNA flap sequence, (14) a Rev responsive element sequence (RRE), (i5) a Woodchuck hepatitis virus posttranscriptional regulatory element sequence, (WPRE), and (i6) a self-inactivating 3′ LTR comprising a deletion in the U3 region that renders the 5′ LTR of the integrated provirus transcriptionally inactive.

By “Rev responsive element sequence” or “RRE” is meant herein a highly structured RNA segment present in the env coding region of unspliced and partially spliced viral mRNAs. In the presence of Rev, the retrovirus mRNAs that contain RRE can be exported from the nucleus to the cytoplasm to be translated and further packaged.

By “central polypurine tract (cPPT)/DNA flap sequence” is meant herein an initiation site, from which, during lentiviral retro-transcription, the DNA synthesis typically starts together with from the polypurine tract (PPT). The plus strand overlap obtained is called the central DNA flap (99 nucleotides), which is known to play a role in enhancing lentiviral provirus nuclear import. In the field of lentivector technology, it is now common knowledge that the introduction of this cis-acting cPPT element in the transfer vector plasmid highly increases the vector transduction efficiency in certain cell types, notably in hematopoietic stem cells, as described for example in Van Maele et at. (2003) J. Virol. 77:4685-4694.

By “Woodchuck hepatitis virus posttranscriptional regulatory element” or “WPRE” is meant herein a DNA sequence that, when transcribed, creates a tertiary structure enhancing expression. The presence of WPRE, in particular in combination with cPPT, thus enables increasing transduction efficiency and transgene expression. By WPRE is also meant herein an improved WPRE, such as the improved WPRE described in Zanta-Boussif et al. (2009) Gene Therapy 16:605-619.

In this particular embodiment, the nucleic acid system according to the invention is preferably located in inverse orientation between the sequences (i4) and (i5).

Accordingly, in a particular embodiment, the retroviral vector of the invention comprises successively:
(11) a modified 5′ LTR comprising a CMV enhancer substituted for the U3 region,
(12) a psi and gag sequence,
(i3) a central polypurine tract (cPPT)/DNA flap sequence,
(14) a Rev responsive element sequence (RRE),
(15) a Woodchuck hepatitis virus posttranscriptional regulatory element sequence (WPRE), and
(16) a self-inactivating 3′ LTR comprising a deletion in the U3 region that renders the 5′ LTR of the integrated provirus transcriptionally inactive,
wherein the nucleic acid system is located in inverse orientation between the sequences (i4) and (i5).

In a particular embodiment, the vector is a lentiviral vector.

In a particular embodiment, (i) the sensor component and (ii) the transducer/effector component of a nucleic acid system of the invention are included in a single lentiviral vector.

In this case, the vector may be considered as an “all-in-one” vector and may be labeled as such throughout the specification. An example of such a vector is provided in Example 2 below.

As such, according to a particular embodiment, the present invention relates to a vector comprising a nucleic acid system comprising:
(i) a sensor component comprising a sequence encoding
(a) at least one extracellular ligand recognition domain;
(b) a transmembrane domain of a B-cell receptor (BCR) or of a T-cell receptor (TCR); and
(c) optionally a signaling domain that controls cell activation of a B-cell receptor or of a T-cell receptor; and
(ii) a transducer/effector component comprising a sequence encoding
(d) the pNR4A1 promoter consisting of SEQ ID NO: 1, or a functional fragment thereof; and
(e) at least one effector protein of interest, and
(iii) an ORF separating sequence placed between the (i) sensor component and the (ii) transducer/effector component,
wherein the (i) sensor component and the (ii) transducer/effector components of the nucleic acid system are both under the control of the pNR4A1 promoter consisting of SEQ ID NO: 1.

Each of the elements (a) to (e) and (iii) may be as those described above.

As such, the at least one effector protein of interest (e) and the elements of the sensor component, (a) the at least one extracellular ligand recognition domain, (b) the transmembrane domain of a B-cell receptor (BCR) or of a T-cell receptor (TCR), and, when present, (c) the signaling domain that controls cell activation of a B-cell receptor or of a T-cell receptor, are all under the control of the pNR4A1 promoter consisting of SEQ ID NO: 1, or a functional fragment thereof.

In particular, the pNR4A1 promoter, or a functional fragment thereof is the only promoter present in the vector. In this case, the pNR4A1 promoter, or a functional fragment thereof controls both the sensor and the effector, which provides an autoregulatory feedback loop.

In a particular embodiment, the at least one effector protein of interest (e) and the elements of the sensor component, (a) the at least one extracellular ligand recognition domain, (b) the transmembrane domain of a B-cell receptor (BCR) or of a T-cell receptor (TCR), and, when present, (c) the signaling domain that controls cell activation of a B-cell receptor or of a T-cell receptor, are all under the control of a single pNR4A1 promoter consisting of SEQ ID NO: 1, or a functional fragment thereof.

In a particular embodiment, the at least one effector protein of interest (e) and the elements of the sensor component, (a) the at least one extracellular ligand recognition domain, (b) the transmembrane domain of a B-cell receptor (BCR) or of a T-cell receptor (TCR), and, when present, (c) the signaling domain that controls cell activation of a B-cell receptor or of a T-cell receptor, and the pNR4A1 promoter consisting of SEQ ID NO: 1, or a functional fragment thereof are all in the same orientation.

According to a particular embodiment, a vector according to the invention may comprise, successively, a pNR4A1 promoter consisting of SEQ ID NO: 1, or a functional fragment thereof, at least one effector protein of interest, an ORF separating sequence, in particular a T2A linker of SEQ ID NO: 18, a sensor component, a Woodchuck hepatitis virus posttranscriptional regulatory element sequence (WPRE) and Long Terminal Repeats (LTRs).

In particular embodiment, a vector according to the invention may comprise, from 5′ to 3′, a pNR4A1 promoter consisting of SEQ ID NO: 1, or a functional fragment thereof, at least one effector protein of interest, an ORF separating sequence, in particular a T2A linker of SEQ ID NO: 18, a sensor component, a Woodchuck hepatitis virus posttranscriptional regulatory element sequence (WPRE) and Long Terminal Repeats (LTRs).

According to another embodiment, the sensor component is included in a first vector, and the transducer/effector component is included in a second vector. Thus, according to a particular aspect, the present text also describes a kit comprising:
a first vector comprising the sensor component
a second vector comprising the transducer/effector component

The vectors according to this embodiment may be those as described above.

In a particular embodiment, the first vector is a retroviral vector, in particular is a lentiviral vector. In a particular embodiment, the second vector is a retroviral vector, in particular is a lentiviral vector. In a particular embodiment, both the first and the second vectors are retroviral vectors, in particular are lentiviral vectors.

In a particular embodiment, in the first vector comprising the sensor component, elements, (a) the at least one extracellular ligand recognition domain, (b) the transmembrane domain of a B-cell receptor (BCR) or of a T-cell receptor (TCR), and, when present, (c) the signaling domain that controls cell activation of a B-cell receptor or of a T-cell receptor, are under the control of the pNR4A1 promoter consisting of SEQ ID NO: 1, or a functional fragment thereof.

### Uses and Methods of Use

### Transformed Cells

A further aspect of the invention relates to a cell which has been transfected, infected or transformed by a nucleic acid, by a vector or by a kit according to the invention.

The term “transformation” means the introduction of a “foreign” (i.e. extrinsic or extracellular) gene, DNA or RNA sequence to a cell, so that the cell will express the introduced gene or sequence to produce a desired substance, typically a protein or enzyme coded by the introduced gene or sequence. A cell that receives and expresses introduced DNA or RNA bas been “transformed”.

The nucleic acid system of the invention may be used to produce one or more effector proteins of interest in a suitable expression system. The term “expression system” means a host cell and compatible vector under suitable conditions, e.g. for the expression of a protein coded for by foreign DNA carried by the vector and introduced to the host cell.

Common expression systems include mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Agl4 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing a nucleic acid system according to the invention, said method comprising the steps of: (i) introducing in vitro or ex vivo, in particular ex vivo, a nucleic acid system, a vector or a kit as described above into a competent host cell, (ii) culturing in vitro or ex vivo, in particular ex vivo, the recombinant host cell obtained and (iii), optionally, selecting the cells which express said system.

Of note, the nucleic acid system according to the invention can also be introduced directly in vivo with viral or DNA vectors.

Such recombinant cells can be transfected in patients in need thereof as part of a therapeutic strategy as presented in the applications further below.

In a particular embodiment, the cell may be selected from B cells and T cells. B cells and T cells are described further above in the specification.

As such, a particular aspect of the invention relates to a B cell or to a T cell which has been transfected by a nucleic acid system, by a vector or by a kit of the invention as described above.

In a preferred embodiment, the B cells and T cells transfected according to the invention are those of the patient to be treated (autologous set up). B cells and T cells of said individual may be collected, transformed ex vivo in order to introduce the nucleic acid system of the invention in their genome, and reintroduced in the individual's body.

In another embodiment, the B cells and T cells transfected according to the invention are those of a donor (allogenic set up).

### Pharmaceutical Composition

A particular subject of the present invention refers to a pharmaceutical composition comprising a nucleic acid system, a vector, a kit or a cell as previously described and a pharmaceutically acceptable vehicle.

“Pharmaceutically” or “pharmaceutically acceptable” refers to molecular entities and compositions that do not produce an adverse, allergic or other untoward reaction when administered to a mammal, especially a human, as appropriate.

A pharmaceutically acceptable vehicle or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the nucleic acid system, vector, kit or cell can be administered in a unit administration form, as a mixture with conventional pharmaceutical supports, to animals and human beings.

Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

### Applications

As mentioned above, the efficacy of the present nucleic acid system may be translated to many pathologies, such as tumors, autoimmune disorders, transplantation rejection, allergies, neurological disorders, and infectious diseases. Indeed, the main advantage of this approach is its full programmability in terms of recognized signal and output functions, which can be adapted to the targeted disease, towards the same goal of continuous sensing of disease-specific biomarkers and triggering of physiological expression of therapeutic molecules in vivo in response.

As used herein, the term “tumor” or “cancer” refers to a condition in which abnormally replicating cells of host origin are present in a detectable amount in a subject. It includes, but is not limited to, solid tumors and blood borne tumors The term cancer or tumor includes diseases of the skin, tissues, organs, bone, cartilage, blood and vessels. The term “cancer” or “tumor” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. The cancer can be a malignant or non-malignant cancer. Cancers include, but are not limited to, biliary tract cancer; brain cancer; breast cancer; cervical cancer; choriocarcinoma; colon cancer; endometrial cancer; esophageal cancer; gastric cancer; intraepithelial neoplasms; leukemias; lymphomas; liver cancer; lung cancer; melanoma; neuroblastomas; oral cancer; ovarian cancer; pancreatic cancer; prostate cancer; rectal cancer; renal cancer; sarcomas; skin cancer; testicular cancer; thyroid cancer; as well as other carcinomas and sarcomas. Cancers can be primary or metastatic.

As used herein, the term “infectious disease” refers to a condition in which an infectious organism or agent is present in a detectable amount in the blood or in a normally sterile tissue or normally sterile compartment of a subject. Infectious organisms and agents include viruses, mycobacteria, bacteria, fungi, and parasites. The terms encompass both acute and chronic infections, as well as sepsis.

In some embodiments, the viral infection comprises infection by one or more viruses selected from the group consisting of Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses. In some embodiments, the viral infection comprises infection by one or more viruses selected from the group consisting of adenovirus, rhinovirus, hepatitis, immunodeficiency virus, polio, measles, Ebola, Coxsackie, Rhino, West Nile, small pox, encephalitis, yellow fever, Dengue fever, influenza (including human, avian, and swine), lassa, lymphocytic choriomeningitis, junin, machuppo, guanarito, hantavirus, Rift Valley Fever, La Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe, pachindae viruses, adenovirus, Dengue fever, influenza A and influenza B (including human, avian, and swine), junin, measles, parainfluenza, Pichinde, punta toro, respiratory syncytial, rhinovirus, Rift Valley Fever, severe acute respiratory syndrome (SARS), Tacaribe, Venezuelan equine encephalitis, West Nile and yellow fever viruses, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and Kyasanur forest disease.

Bacterial infections that can be treated according to this invention include, but are not limited to, infections caused by the following: Staphylococcus; Streptococcus, including S. pyogenes; Enterococcl; Bacillus, including Bacillus anthracis, and Lactobacillus; Listeria; Corynebacterium diphtheriae; Gardnerella including G. vaginalis; Nocardia; Streptomyces; Thermoactinomyces vulgaris; Treponerna; Camplyobacter, Pseudomonas including aeruginosa; Legionella; Neisseria including N. gonorrhoeae and N. meningitides; Flavobacterium including F. meningosepticum and F. odoraturn; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. fickettsfi, Chlamydia including C. psittaci and C. trachornatis; Mycobacterium including M. tuberculosis, M. intracellulare, M. folluiturn, M. laprae, M. avium, M. bovis, M. africanum, M. kansasii, M. intracellulare, and M. lepraernurium; and Nocardia.

Protozoa infections that may be treated according to this invention include, but are not limited to, infections caused by leishmania, kokzidioa, and trypanosoma.

A complete list of infectious diseases can be found on the website of the National Center for Infectious Disease (NCID) at the Center for Disease Control (CDC) (World Wide Web (www) at https://www.cdc.gov/DiseasesConditions/), which list is incorporated herein by reference. All of said diseases are candidates for treatment using the nucleic acid system according to the invention

As used herein, the term “autoimmune disease” refers to the condition when the body's natural defense system can't tell the difference between its own cells and foreign cells, causing the body to mistakenly attack normal cells.

Autoimmune diseases that may be treated according to the invention include type 1 diabetes, rheumatoid arthritis (RA), psoriasis or psoriac arthritis, multiple sclerosis, systemic lupus erythematosus (SLE), inflammatory bowel disease, Addison's disease, Graves' disease, Sjagren's syndrome, Hashimoto's thyroiditis, Myasthenia gravis, autoimmune vasculitis, pernicious anemia and celiac disease.

As used herein, the term “transplantation rejection” occurs when transplanted tissue is rejected by the recipient's immune system, which destroys the transplanted tissue. Transplant rejection can be lessened by determining the molecular similitude between donor and recipient and by use of immunosuppressant drugs after transplant. Use of a nucleic acid system according to the invention can allow to reprogram B cells and T cells of the graft recipient so that they no longer recognize the cells of the graft as foreign cells to be destroyed.

As used herein, the term “allergy” or “allergic disease” refers to a number of conditions caused by hypersensitivity of the immune system to typically harmless substances in the environment. Allergen immunotherapy involves exposing people to larger and larger amounts of allergen in an effort to change the immune system's response. Similarly, use of a nucleic acid system of the invention would allow overcoming an individual's sensitivity to allergens by reprograming B and T cells to no longer recognize these substances as foreign.

Examples of allergy types include allergic rhinitis, asthma, atopic eczema, anaphylaxis, insect venom, drug allergies and food allergies.

As used herein, the term “neurological disorder” refers to any disorder of the nervous system. They may be caused by faulty genes or due to problems with the way the nervous system develops, they may be degenerative diseases, where the nerve cells are damages or die, they may be due to diseases of the blood vessels that supply the brain, to injuries, to seizure disorders, to cancer or to infections. Use of a nucleic acid system according to the invention can allow to reprogram B cells and T cells to locally secrete molecules that will promote the restoration of destroyed tissues or restore a correct neuron signaling for instance.

Neurological disorders include, but are not limited to Acute Spinal Cord Injury, Alzheimer's Disease, Amyotrophic Lateral Sclerosis (ALS), Ataxia, Bell's Palsy, Brain Tumors, Cerebral Aneurysm, Epilepsy and Seizures, Guillain-Barré Syndrome, Headaches, Head Injuries, Huntington's disease, Hydrocephalus, Lumbar Disk Disease (Herniated Disk), Meningitis, Multiple Sclerosis, Muscular Dystrophy, Neurocutaneous Syndromes, Parkinson's Disease, Spina Bifida, Strokes (Brain Attack), Cluster Headaches, Tension Headaches, Migraine Headaches, Encephalitis, Septicemia, and Myasthenia Gravis.

According to a particular aspect, the present invention relates to a method for preventing and/or treating a tumor, an infectious disease, an immune disorder, a transplantation rejection, and/or an allergy comprising at least a step of administering a nucleic acid system, a vector, a kit, a cell, or a pharmaceutical composition as described herein to an individual in need thereof.

As used herein, the term “individual” or “subject” is a mammal, most preferably a human. Mammals include, but are not limited to, domesticated animals (e.g., cows, sheep, cats, dogs, and horses), primates (e.g., humans and non-human primates such as monkeys), rabbits, and rodents (e.g., mice and rats). Most preferably, the individual or subject is a human. In particular “an individual in need thereof” is an individual suffering from a tumor, an infectious disease, an immune disorder, a transplantation rejection, and/or an allergy.

The invention further relates to a nucleic acid system, a vector, a kit, a cell, or a pharmaceutical composition as described herein for use as a medicament.

Another aspect of the invention relates to a nucleic acid system, a vector, a kit, a cell, or a pharmaceutical composition as described herein for its use in the prevention and/or the treatment of a tumor, an infectious disease, an immune disorder, a transplantation rejection, and/or an allergy.

The invention also relates to the use of a nucleic acid system, a vector, a kit, a cell, or a pharmaceutical composition as described herein for the manufacture of a medicament for the treatment of a tumor, an infectious disease, an immune disorder, a transplantation rejection, and/or an allergy.

It is to be understood that the disclosure encompasses all variations, combinations, and permutations in which at least one limitation, element, clause, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The following examples are provided for purpose of illustration and not limitation.

### EXAMPLES

### Examples

### Example 1: Nucleic Acid System of the Invention in Reprogramming B and T Cells for Better Control and Regulation of the Immune Response

### Materials & Methods

### Construction of Plasmids Encoding the Nucleic Acid System Components.

The NR4A1 promoters were amplified from human genomic DNA and cloned into the pHRSN vector within EcoRI and BamHJ restriction sites. The following primers of sequences set forth as SEQ ID NO: 6 to 9 and 22 to 23 were used in combination with the primer having the sequence set forth as SEQ ID NO: 10:

| SEQ ID | | |
|---|---|---|
| NO: | NAME | SEQUENCE (5′-3′) |
| 6 | −2095 | GATACGAATTCCAGTTCAAACGTGTTGTTCAAGGG |
| | PrNR4A1 | |
| | EcoRI | |
| 7 | −1641 | GATACGAATTCCAGGAGCAGAGATTAGGTGGCA |
| | PrNR4A1 | |
| | EcoRI | |
| 8 | −1142 | GATACGAATTCCAGGTGATAACTGGTCAGAGCTG |
| | PrNR4A1 | |
| | EcoRI | |
| 9 | −625 | GATACGAATTCTGTGCACTAGCTGCGCCTA |
| | PrNR4A1 | |
| | EcoRI | |
| 10 | +109 | GATACGGATCCGGTCCCGCGTGCGCT |
| | PrNR4A1 | |
| | EcoRI | |
| 19 | −499 | GATCgaattcCAGGAAGGGCTTGGGAAGGTGTAAAG |
| | PrNR4A1 | |
| | EcoRI | |
| 20 | −306 | GATCgaattcAGCTGGGACCCGAGTCCGGT |
| | PrNR4A1 | |
| | EcoRI | |
| 21 | −60 | GATCgaattcTTGTATGGCCAAAGCTCGACGGGGATACGGATCC |
| | PrNR4A1 | GGTCCCGCGTGCGCT |
| | EcoRI | |

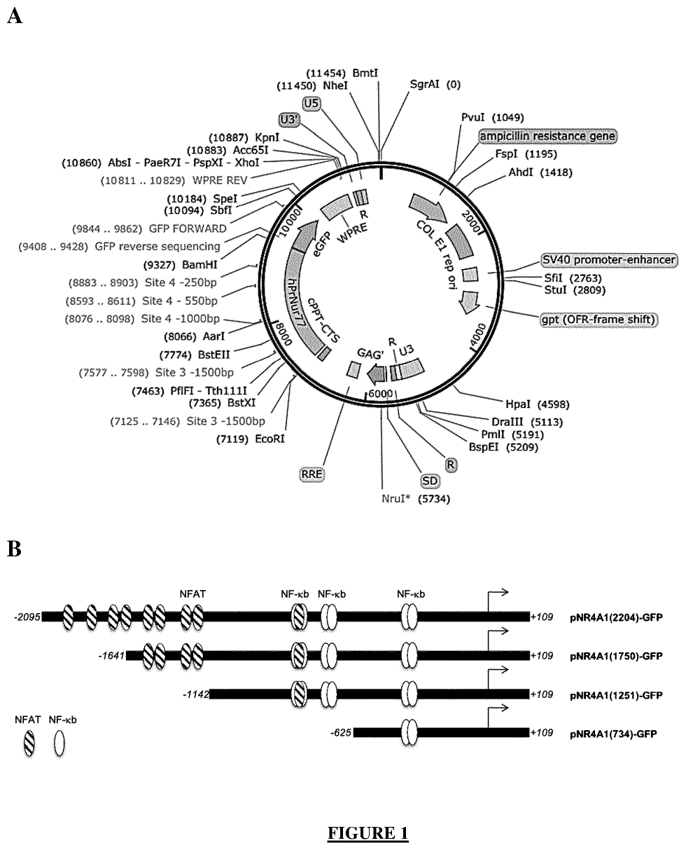

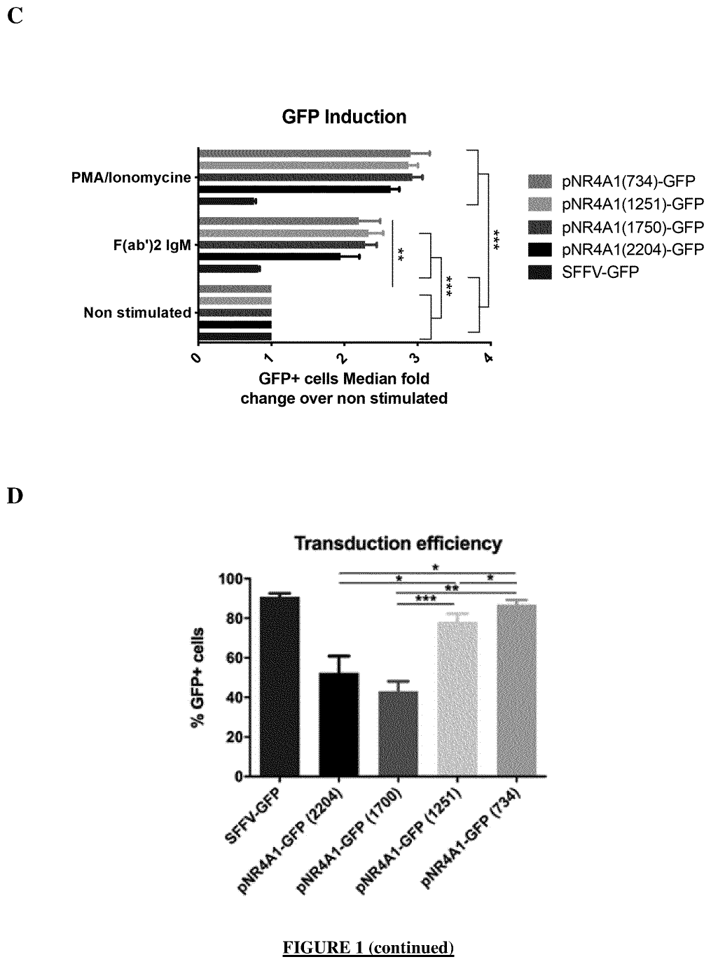

The schematic representation of the lentiviral vector comprising the pNR4A1(2204), the pNR4A1(1750), the pNR4A1(1251) or the pNR4A1(734) fragments are included in FIG. 1A.

The ectopic BCRs were cloned by inserting variables regions of monoclonal antibodies (OBI sequence and ADRI sequence) synthesized by Genscript into the published FAM0 construct.

The destabilized Turbo GFP was a kind gift from Dr. Mangeot. They were cloned in between BamHI and SbfI restriction sites after the promoters to replace GFP in constructions pNR41(608) and pNR4A1(415).

### Cell Lines and Primary Cells.

Jurkat cells (ACC-282), Namalwa cells (Burkitt lymphoma (BL) subtype PNT (ACC-69), Raji (ACC-319) and BL-2 (ACC-625) were purchased from DSMZ, Braunschweig, Germany). BJAB (ACC-757) and Ramos (ACC-603) cells were a kind gift from Pr. Belot and Dr. Gruffat. B cell lines were grown in culture flasks with RPMI-1640 containing 50 g/ml of penicillin, and streptomycin supplemented with 10% to 20% heat inactivated fetal bovine serum at 37° C. in a humidified atmosphere of 95% air/5% CO2 as recommended on the DSMZ website. 293T (human kidney epithelial cells) were grown in Dulbecco's modified Eagle medium (Gibco, Invitrogen) medium supplemented in the same manner.

Peripheral Blood MorphoNuclear cells (PMBCs) were isolated from human donor peripheral blood by human Ficoll-gradient. Human primary B cells were then isolated by a positive selection with anti-CD19-conjugated magnetic beads (Miltenyi). They were grown in complete RPMI-1640 medium supplemented with 2 mM glutamine, 100 IU/ml penicillin, 100 mg/ml streptomycin, 55 M B-ME, 1% HEPES at 1×106 cells/ml. Cross-link CD40L (2 μg/ml,), TL-4 (2 ng/ml,) and BAFF (10 ng/ml) were added to the media.

### LV Titration.

Infectious titters (TU/ml) of lentivirus (LV) were quantified by adding serial dilutions of the LVs to 293T target cells. Ten-days after transduction, genomic DNA was extracted from target cells for qPCR analysis of viral genome copy number. The quantitative PCR was performed using 5 μl of DNA on a StepOnePlus system with specific primers for detection of the integrated LV backbone. The titters were normalized to human actin gene copies. Two control samples were processed in parallel for each run.

### LV Production and B Cell Transduction.

LVs respectively encoding the described constructs were generated by transient transfection of 293T cells through calcium phosphate precipitation. For pseudotyping with the VSV-G and BRL glycoproteins, 2.7 g and 7 g of envelope plasmid were respectively transfected together with a gagpol packaging plasmid (8.6 g) and the plasmid encoding the LV construct (8.6 g). Eighteen hours after transfection, the medium was replaced by Opti-MEM supplemented with 10 mM Hepes and 1% PenStrep. Viral supernatants were harvested 48 h after transfection and filtered on 40 μm-filters. Low-speed concentration was performed by overnight centrifugation of the viral supernatants at 3,000 g at 4° C.

For in vitro transduction of B cell lines, cells were cultured in supplemented RPMI and transduced at a MOI of 10. For primary human B cells transduction, protamine sulfate was added in the media (8 g/ml).

### Cell Stimulation.

1.5×10⁵ to 2.0×10⁵ B cells were stimulated in 96-well plates in supplemented RPMI-1640 media with antibodies or immunostimulants during the indicated times. Stimuli included CpG oligodeoxynucleotides (ODNs) 2006 for B cells (InvivoGen), LPS from Escherichia coli (Sigma-Aldrich), anti-human F(ab′)2 IgM and F(ab′)2 IgG (Southern Biotech), ionomycin (EMD millipore), and PMA (Cell Signaling Technology). After stimulation, cells were washed and resuspended in PBS before flow cytometry acquisition. For antigen-specific stimulation, 100 antigen-coated beads per B cell were added in the supernatant.

For Jurkat T cells stimulation, 2×10⁶ cells were plated in 96-well plates and stimulated with 1 g/mL of CD3/CD8 antibodies (Invitrogen) or with TransAct (Mylteniy Biotech) according to the manufacturer's instructions. For kinetics assessments, to remove stimulant molecules, plates were washed three times with 200 μl of PBS.

Generation of Synthetic Particulate Antigen Beads Coated with Ovalbumin.

Synthetic particulate antigens (SPAgs) have been generated as previously described. Briefly, 0.4 μm-Flashred streptavidin beads were incubated with monobiotinylated ovalbumin (OVA, Sigma) before being washed twice with PBS containing 2% BSA and filtered through 0.1 μm and 0.65 μm columns (Durapore, Merck Millipore) to remove respectively unbound molecules and beads aggregates. Beads concentration was assessed with a standard curve by reading FlashRed fluorescence on a Tecan plate reader.

### Internalization Assay.

1×10⁵ cells were plated in 96-well plates and incubated for 1 h, 6 h, 24 h or 48 h with 100 SPAgs-OVA per B cells. After incubation, cells were washed in PBS 2% SVF before surface staining with an anti-OVA antibody.

### Confocal Microscopy Analysis.

B cells loaded with SPAg were cultured overnight. 1×10⁶ B cells were plated on 17 mm glass coverslips (Zeiss) preincubated 4 hours with 0.01% poly-L-lysine (Sigma). Cells were permeabilized with 0.1% triton, then incubated for 30 minutes at room temperature with blocking solution (PBS-1% BSA) and stained with eFluor570 anti-B220 (clone RA3-6B2, BD) and AlexaFluor 488-conjugated anti-LAMP1 (clone H4A3, BD) mAbs for 1 hour at room temperature. After 3 washes with PBS, cells were stained with Hoechst (1/10′000) for 5 minutes. After 3 additional washes, coverslips were mounted on glass slides with mowiol mounting medium. Confocal 3D image stacks were acquired with confocal spectral LM610 (Zeiss). Images were analyzed with FIJI software.

### Flow Cytometry Staining and Analysis.

For staining, 2×10⁵ cells were resuspended in PBS containing 2% FCS and incubated with an optimal dilution of fluorochrome conjugated antibodies anti-IgM-APC (Miltenyi), anti-IgG-PE (Miltenyi) anti-CD86-VB (Miltenyi), anti-HLA-DR-APC-Vio7 (Miltenyi) anti-IFNg-APC (Miltenyi) and anti-OVA-FITC (Cell signaling) during 30 min a 4° C. before being washed with complemented PBS.

Data were acquired on the FACSCantoII (BD Biosciences) and analyzed on FlowLogic™.

### Cellular Extract Preparation and Western Blotting.

The following antibodies were used: anti-IgM (LsBio), anti-IgG (LsBio60606, Cliniscience, France.) and anti-calnexin (SPA-860, Stressgen Biotechnologies, Canada). Approximatively 2×10⁶ cells were lysed in cell lysis buffer containing 20 mM Tris-HCl (pH 8.0), 1% NP-40, 0.1% sodium deoxycholate, 0.1% SDS, 150 mM NaCl and 1 mM PMSF. Proteins were heated 3 min at 95° C. in denaturing buffer, separated by 12% SDS-PAGE under reducing conditions and revealed by Western blotting using the HRP revelation kit (IgG, IgM and calnexin) or the Odyssey machine (IL-18).

### Statistical Analysis.

Statistical significance was evaluated using Prism software. Data are expressed as means±standard mean error and differences were considered as significant (P<0.05; *); very significant (P<0.01; **) and highly significant (P<0.001; ***).

### Results

### Generation of a Small BCR-Inducible Promoter Construct.

The Nur77 (NR4A1) protein has been shown to be specifically induced in B cells following BCR stimulation (Ashouri et al.). Based on this observation, the inventors isolated several fragments of the NR4A1 promoter ending at the transcription starting site with decreasing sizes and placed them upstream the GFP reporter gene in a lentiviral vector (FIG. 1B) or upstream the destabilized Turbo GFP gene. Of note, these fragments may contain binding sequences for the NFAT and NF-kB transcription factors that are involved in the BCR signaling cascade. The fragments are as follow:

| | Length of | | Location of the fragment vis à vis |
|---|---|---|---|
| Fragment name | the fragment | | the start codon of the Nur77 gene |
| pNR4A1(2204)-GFP | 2204 | bp | −2095; +109 |
| pNR4A1(1750)-GFP | 1750 | bp | −1641; +109 |
| pNR4A1(1251)-GFP | 1251 | bp | −1142; +109 |
| pNR4A1(734)-GFP | 734 | bp | −625; +109 |
| pNR4A1(608)-GFP | 608 | bp | −499; +109 |
| pNR4A1(415)-GFP | 415 | bp | −306; +109 |
| pNR4A1(169)-GFP | 169 | bp | −60; +109 |

To assess the effect of BCR stimulation on promoter inducibility, BJAB cells, a B cell line, were transduced by lentiviral vectors encoding either the inducible reporter constructs or a constitutively active (Spleen Focus Forming Virus, SFFV) (control) promoter prior to 24 h-stimulation of the endogenous IgM BCR (Gagnepain et al.).

For all the constructs except pNR4A1(169)-GFP, an increase in GFP or destabilized Turbo GFP expression was observed.

For constructs pNR4A1(2204)-GFP, pNR4A1(1750)-GFP, pNR4A1(1251)-GFP and pNR4A1(734)-GFP, an increase around 2.5-fold in GFP expression was observed and was in the same range as the positive control with ionomycin combined to PMA, which has been shown to activate the BCR signaling cascade without requiring BCR direct linkage (FIG. 1C). The ectopic promoter length did not impact the magnitude of the induction following BCR stimulation, but played a key role in the percentage of transduction, which increased along with decreasing size of the promoter, the 734-bp construct leading to more than 90% of transduction (FIG. 1D).

While looking for the minimal sequence of pNR4A1 which retained promoter induction by the BCR, the inventors found that a fragment reduced down to 415-bp on the 5′ exhibited an induction similar to the 734-bp construct.

Of note, no induction was observed for the 169-bp fragment, suggesting that key regulatory sequences are present between positions −305 and −60.

Given the similar inducibility of the constructs and taking in account the limited packaging capacity of lentiviral vectors, the inventors decided to focus on the smaller promoter construct (734 bp) for subsequent experiments.

### Characterization of the Inducible 734 bp NR4A1 Promoter.

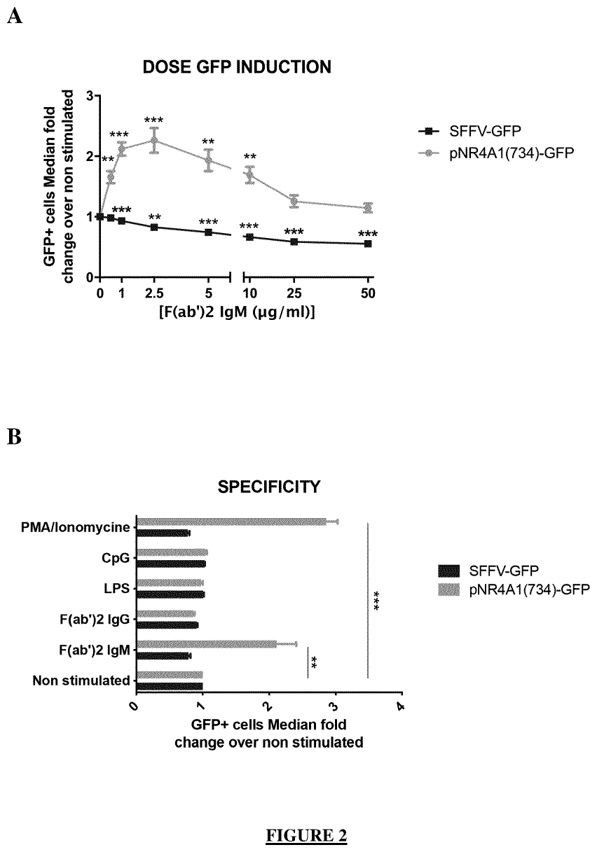

The dose response of the promoter after 24 h of stimulation with F(ab′)2 IgM molecules was monitored and peaked for a concentration of 2.5 g/ml (FIG. 2A).

Importantly, the inventors showed that the NR4A1 reporter construct was specifically induced after antigen receptor signaling, but not after TLR-4 (LPS), and TLR-9 (CpG) stimulation in B cells (FIG. 2B). All previous experiments were carried out in BJAB cells, but the inducibility of the promoter was also assessed in other B cell lines that express IgM BCRs. Similar to BAJB cells, a 2.5-fold induction of the promoter was observed in BL-2 cells after BCR linkage but not in other cell lines, where an increase in GFP expression was only detected after culture with PMA and ionomycin.

### Induction, Extinction and Reversibility Kinetics of the Promoter.

In order to refine the promoter kinetics, the GFP reporter placed after the constitutive or inducible promoters was replaced by the destabilized TurboGFP, which has a shorter maturation time and a shorter half-life. First, the kinetics of promoter induction were assessed after 0 to 24 h of continuous stimulation.

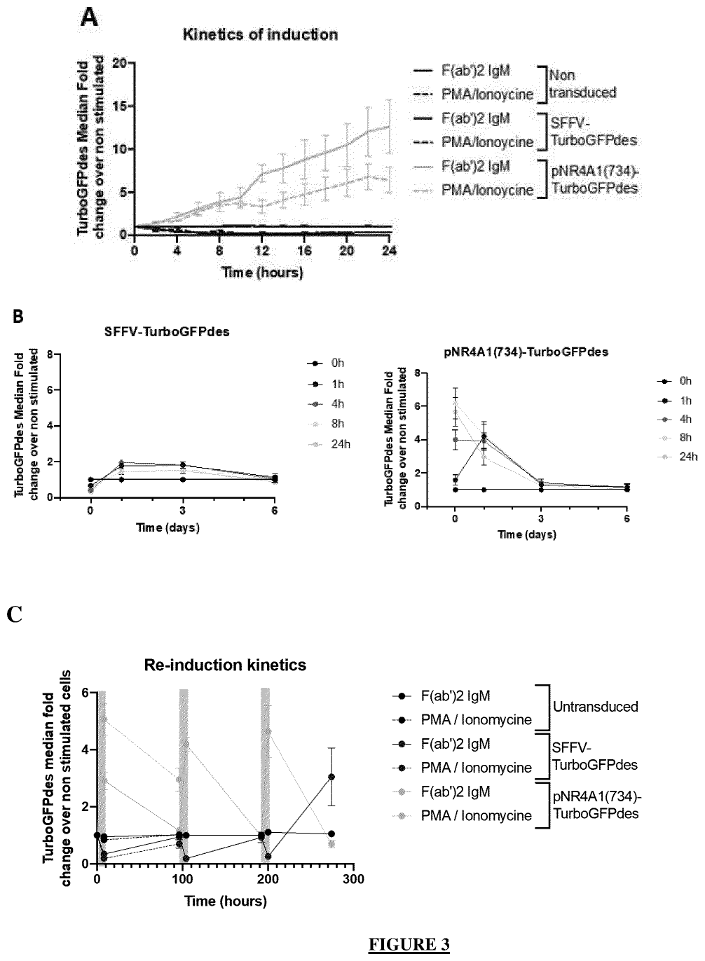

The induction was really fast as after 4 h, a two-fold increase in TurboGFPdes was already observed and this induction reached 12-folds after 24 h of stimulation, demonstrating the fast response of the promoter (FIG. 3A). Alternatively, the extinction kinetics were also investigated to see if this induction could be reversed when the stimulus is removed. To address this question, cells transduced with the TurboGFPdes under the control of the constitutive or inducible promoters were stimulated through their BCRs for 1, 4, 8 or 24 h before removing the inducer molecules. Three days after stimuli removal, the TurboGFPdes median regained the basal value of non-stimulated cells and remained stable for 10 days, demonstrating the reversibility of the induction (FIG. 3B).

However, the inventors then wanted to know if this system could be induced multiple times and if it is still completely reversible after several rounds of induction. Three rounds of stimulation were performed on transduced by alternating 8 h of stimulation (grey) and 80 h of rest (white). The induction-fold slightly increased over stimulation rounds from 3 to 5 as normalized by unstimulated cells. Importantly, this induction was fully reversible even after 3 cycles of stimulation/rest, which is of particular interest from a therapeutic point of view to treat disease flares in chronic patients (FIG. 3C).

### Construction of a Vector Encoding a Membrane-Anchored BCR Receptor.

After validating the BCR inducible promoter, the inventors then attempted creating a complete synthetic circuit, i.e. a nucleic acid system of the invention, to reprogram B cells. Toward this goal, a specific sensor (a membrane-anchored BCR) to control promoter induction was developed. This membrane-anchored BCR ‘sensor’ was encoded by a lentiviral vector containing the variable regions of monoclonal antibodies directed either against the HbS protein of the Hepatitis B virus (FAM0-ADRI) (Cerino et al.) or against the ovalbumin (OVA) protein (FAM0-OVA) (Dougan et al.) along with the constant IgG/kappa human immunoglobulin domains fused to the transmembrane IgG domains. Of note, the intronic regions that allow the conditional secretion of immunoglobulins upon B cell activation were removed in order to express only the membrane-anchored form of the immunoglobulin (Fusil et al.). The nucleic sequence of the FAM0-OVA construct is provided in SEQ ID NO: 11. In this construct, the variable regions of the monoclonal antibody directed against the ovalbumin (OVA) protein is materialized in bold in the sequence of SEQ ID NO: 11 (from the nucleotide in position 561 to the nucleotide in position 3149).

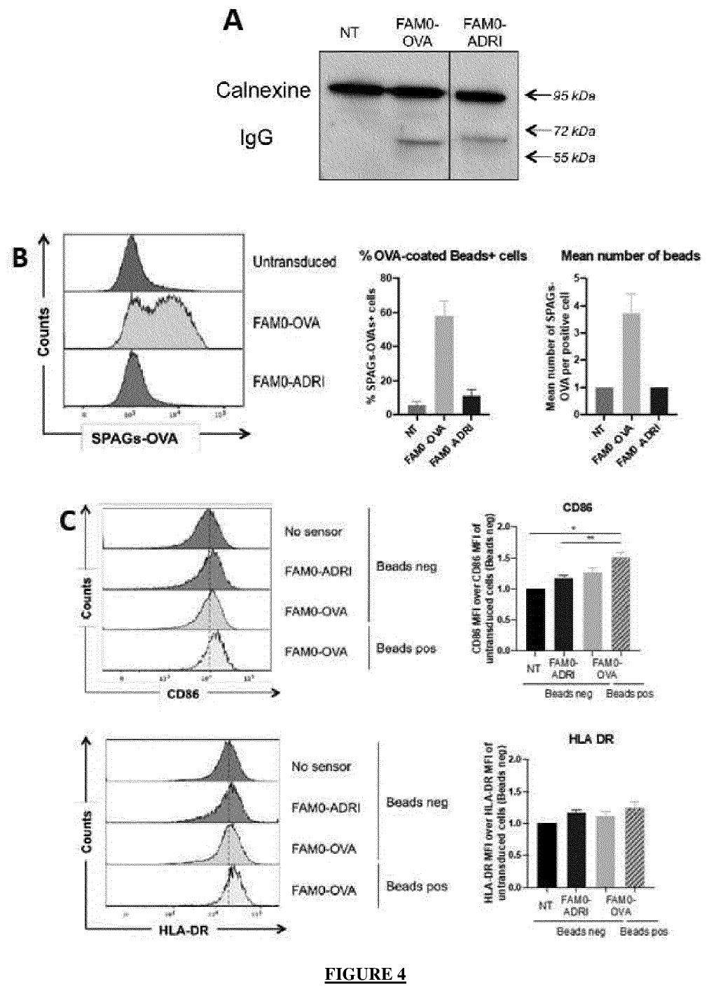

To validate this circuit component, both in terms of expression and functionality, lentiviral vectors encoding sensors were used to transduce BJAB cells. BJAB cells endogenously express IgM immunoglobulins but are negative for IgG immunoglobulins. After transduction, IgG immunoglobulin were detected by western blot (FIG. 4A) and surface cytometry staining, validating the membrane expression of the sensor component. Synthetic particulate antigen beads (SPAGs) composed of 400 nm fluorescent beads coated with OVA molecules were incubated with transduced cells to validate antigen-specific recognition by the sensors. After 24 h of SPAGs-OVA beads incubation, only cells transduced with the OVA-specific sensor were positive for SPAGs-OVA (around 50% of beads positive cells detected by cytometry). The percentage of the SPAGs-OVA positive cells as well as the mean number of beads per positive cells were assessed by immunofluorescence staining and were respectively around 50% and 4 for the FAM0-OVA condition, which is consistent with the cytometry analysis (FIG. 4B).

Of note, a small non-specific binding was observed for non-transduced cells and cells transduced with the FAM0-ADRI construct (less than 5% positive cells and with only one SPAG-OVA beads). To validate sensor functionality and signaling after antigen-specific recognition, the expression of activation markers was quantified by cytometry in sensor transduced cells after 24 h of incubation with SPAGs-OVA beads (FIG. 4C). CD86 was upregulated in cells expressing the OVA sensor as compared to non-transduced cells, suggested an antigen-specific activation.

Unexpectedly, no up-regulation of HLA-DR (MHC-II) was observed after antigen ligation on FAM0-OVA sensors. Indeed, following antigen recognition, the BCR-antigen complex naturally undergoes endocytosis and intracellular processing, leading the antigen presentation on MHC-II complexes (FIG. 4C).

The percentage of cells positive for beads didn't increase over incubation time.

Assembly of Whole Ectopic Components into a Functional Synthetic Circuit.

Moving a step forward, all the circuit components, namely the sensor (membrane-anchored BCR), the transducer (the 734 bp NR4A1 inducible promoter) and the effector (here TurboGFPdes) were introduced together in BJAB by double transduction with two lentiviral vectors encoding respectively the sensor and the TurboGFPdes under the control of the BCR-inducible promoter or the SFFV constitutive promoter as a control. Double transduced cells were then stimulated through their ectopic sensor with anti-IgG molecules and a specific up-regulation of TurboGFPdes expression was observed following this stimulation in cells transduced with sensors and the BCR-inducible promoter. Of note, the slight decrease of activation in cells transduced with the inducible promoter along with sensors compared to cells without sensors following endogenous IgM BCR stimulation was correlated with a reduction of TgM expression. Indeed, the ectopic expression of a new BCR has already been shown to decrease the expression of the endogenous BCR probably due to a competition in the addressing process.

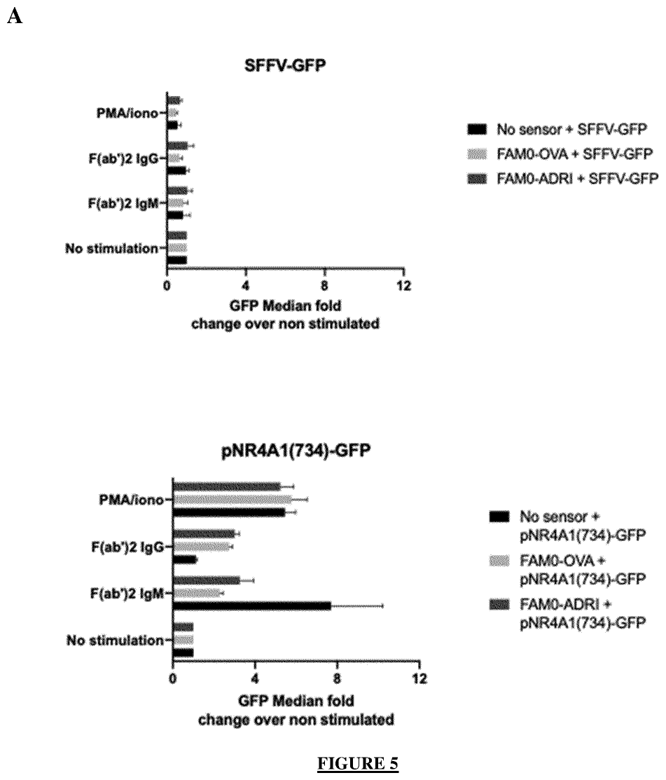

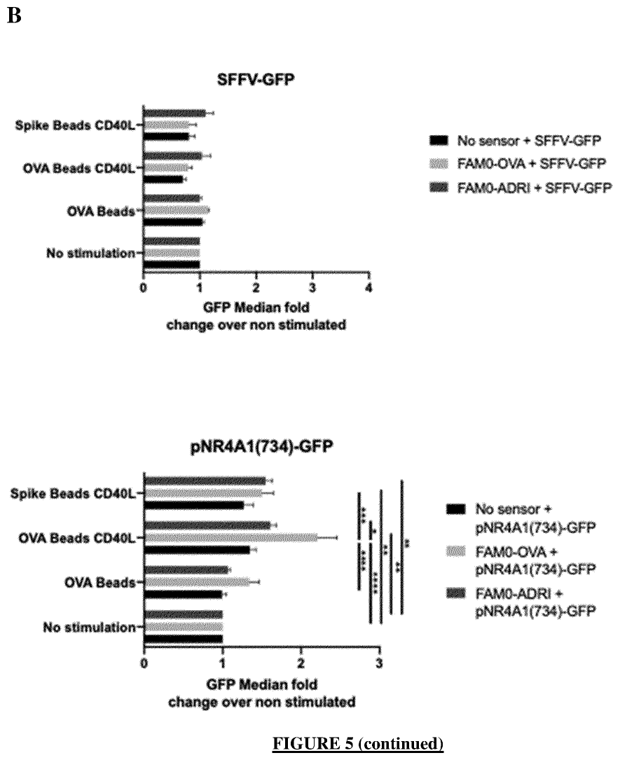

No differences were observed in cells having the SFFV promoter after IgG stimulation (FIG. 5A).

The inventors then wanted to validate the antigen-specific induction of the whole nucleic acid system by using OVA coated beads alone or combined to the CD40L costimulatory molecule to stimulate double transduced cells. After 24 h of stimulation with OVA coated beads plus CD40L, the whole system was specifically activated (threefold) in an antigen-specific manner as no activation was detected following stimulation with Spike RBD coated beads (FIG. 5B).

### Inducibility of the Promoter in T Cells.

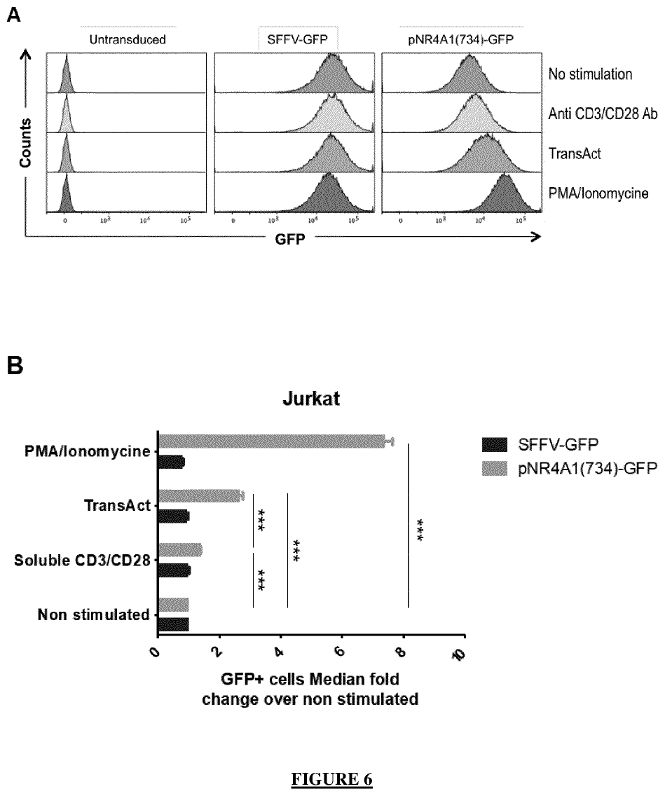

Finally, the inventors tested the 734 bp NR4A1 reporter construct in Jurkat T cells. After 24 h of TCR stimulation with CD3 and CD28 coated beads, a 3-fold amplification in GFP expression was observed (FIGS. 6A and 6B), showing the inducibility of the promoter by the TCR in T cells, which suggests that it should also be activated by CAR constructs.

### Conclusion

Overall, the results presented above demonstrate the efficacy of a nucleic acid system of the invention in reprogramming B and T cells in order to better control and regulate the immune response. Upon binding of target molecules on the dedicated sensor (targeting a given pathological signal), the transducer pNR4A1 is specifically activated leading to the expression of the effector therapeutic molecules placed under its control.

### Example 2: Development of a Self-Amplifying all-In-One Vector

To transpose the invention towards a clinical setting that based on human primary B cells, the inventors developed an ‘all-in-one vector’ encoding all components within the same LV construct. Indeed, the efficiency of transduction of human primary B cells is improved, by implementing all-in-one LVs considering that, as shown below, they allow increasing the number of cells co-expressing all circuit components, thus reducing variability.

### Materials & Methods

### Construction of Plasmids Encoding the Synthetic Circuit Components.

The NR4A1 promoter fragments were amplified from human genomic DNA and cloned into the pHRSIN vector (Demaison, Christophe, Kathryn Parsley, Gaby Brouns, Michaela Scherr, Karin Battmer, Christine Kinnon, Manuel Grez, et Adrian J. Thrasher. «High-Level Transduction and Gene Expression in Hematopoietic Repopulating Cells Using a Human Imunodeficiency Virus Type 1-Based Lentiviral Vector Containing an Internal Spleen Focus Forming Virus Promoter». Human Gene Therapy 13, no 7 (mai 2002): 803-13) within EcoRI and BamHI restriction sites, to replace the SFFV promoter initially controlling a GFP transgene. The following primers, used in example 1, were used:

| SEQ ID | | |
|---|---|---|
| NO | NAME | SEQUENCE (5′-3′) |
| 9 | −625 PrNR4A1 | 5′-GATACGAATTCTGTGCACTAGCTGCGCCTA- |
| | EcoRI | 3′ |
| 10 | +109 PrNR4A | 5′-GATACGGATCCGGTCCCGCGTGCGCT-3′ |
| | BamHI | |

The destabilized Turbo GFP was a kind gift of Dr. Mangeot and was cloned between BamHI and SbfI restriction sites after the promoters to replace GFP in the modified pHRSIN vectors above mentioned.

Fragments encoding the synthetic circuit transgenes for the self-amplifying vector were ordered from Genscript before being inserted by restriction cloning into in the original pHRSIN vector.

### Cell Lines and Primary Cells.

BJAB (ACC-757) cells, originating from Burkitt lymphomas, were a kind gift of Pr. Belot. B cell lines were grown in culture flasks in RPMI-1640 medium containing 50 μg/ml of penicillin and streptomycin supplemented with 10% to 20% heat-inactivated fetal calf serum (FCS) at 37° C. in a humidified atmosphere of 95% air/5% CO2 as recommended on the DSMZ website. 293T cell (human kidney epithelium) were grown in Dulbecco's modified Eagle medium (Gibco, Invitrogen) medium supplemented with 10% FCS.

### Lentiviral Vectors (LVs) Production and B Cell Transduction.

LVs were generated by transient transfection of 293T cells through calcium phosphate precipitation. For pseudotyping of LVs with the VSV-G glycoprotein, 2.7 g of envelope plasmid was transfected together with 8.6 g of gagpol packaging plasmid (psPAX2, Addgene plasmid #12260) and a plasmid encoding the LV construct (8.6 g). Eighteen hours after transfection, the medium was replaced by Opti-MEM supplemented with 10 mM Hepes and 1% PenStrep (Gibco). Viral supernatants were harvested 48 h after transfection and filtered on 40 μm-filters. Low-speed concentration was performed by overnight centrifugation of the viral supernatants at 3,000 g at 4° C.

### LV Titration.

LVs were titrated by adding serial dilutions of the LVs to 293T target cells. Ten-days after transduction, genomic DNA was extracted from target cells (Macherey Nagel) for qPCR analysis of viral genome copy number. The quantitative PCR was performed using 5 μl of DNA on a StepOnePlus system with specific primers for detection of the integrated LV: primer F: 5′-TGT GTG CCC GTC TGT TGT GT (SEQ ID NO: 12), primer R: 5′-GAG TCC TGC GTC GAG AGA GC (SEQ ID NO: 13), and probe 5′-CAG TGG CGC CCG AAC AGG GA (SEQ ID NO: 14). Genomic vector copies in each sample were normalized to human actin gene copies using specific primers: primer F: 5′ TCC GTG TGG ATC GGC GGC TCC A (SEQ ID NO: 15), primer R 5′-CTG CTT GCT GAT CCA CAT CTG (SEQ ID NO: 16), and probe CCT GGC CTC GCT GTC CAC CTT CCA (SEQ ID NO: 17). as previously described (Fusil, Floriane, Sara Calattini, Fouzia Amirache, Jimmy Mancip, Caroline Costa, Justin B Robbins, Florian Douam, et al. «A Lentiviral Vector Allowing Physiologically Regulated Membrane-Anchored and Secreted Antibody Expression Depending on B-Cell Maturation Status». Molecular Therapy 23, no 11 (novembre 2015): 1734-47). The titers were normalized to human actin gene copies. Two control samples were processed in parallel for each run.

### B Cell Transduction.

For in vitro transduction of B cell lines, cells were cultured in supplemented RPMI-1640 medium and transduced at a MOI of 10.

### Cell Stimulation.

1.5×10⁵ to 2.0×10⁵ B cells were stimulated in 96-well plates in supplemented RPMI-1640 medium with antibodies or immunostimulants during the indicated times. Stimuli included anti-human F(ab′)2 IgM and F(ab′)2 IgG (Southern Biotech), ionomycin (EMD millipore), PMA (Cell Signaling Technology) and cross-linked human CD40L (Miltenyi). After stimulation, cells were washed and resuspended in PBS before flow cytometry analysis. For antigen-specific stimulation, 100 antigen-coated beads per B cell were added in the supernatant.

Generation of Synthetic Particulate Antigen (SPAG) Beads Coated with Ovalbumin.

SPAG beads were generated as previously (Sicard, Antoine, Alice Koenig, Stéphanie Graff-Dubois, Sébastien Dussurgey, Angéline Rouers, Valdrie Dubois, Pascal Blanc, et al. «B Cells Loaded with Synthetic Particulate Antigens: A Versatile Platform To Generate Antigen-Specific Helper T Cells for Cell Therapy». Nano Letters 16, no 1 (13 janvier 2016): 297-308).

Briefly, 0.4 μm-Flashred streptavidin beads (Bangslabs) were incubated with monobiotinylated ovalbumin (OVA, Sigma) or Spike RBD (Miltenyi) before being washed twice with PBS containing 2% BSA and filtered through 0.1 μm and 0.65 μm columns (Durapore, Merck Millipore) to remove respectively unbound molecules and bead aggregates. Beads concentration was assessed with a standard curve by reading FlashRed fluorescence on a Tecan plate reader.

### Flow Cytometry Staining and Analysis.

For staining, 2×10⁵ cells were resuspended in PBS containing 2% FCS. The following fluorochromes were used: anti-IgM-APC (Miltenyi), anti-IgG-PE (Miltenyi), anti-CD86-VB (Miltenyi), anti-HLA-DR-APC-Vio7 (Miltenyi) and anti-OVA-FITC (Cell Signaling Technology)

Data were acquired on the FACSCantoII (BD Biosciences) and analyzed on FlowLogic™.

### Statistical Analysis.

Statistical significance was evaluated using Prism software. Data are expressed as means±standard mean error and differences were considered as significant (P<0.05; *); very significant (P<0.01; **) and highly significant (P<0.001; ***).

### Results

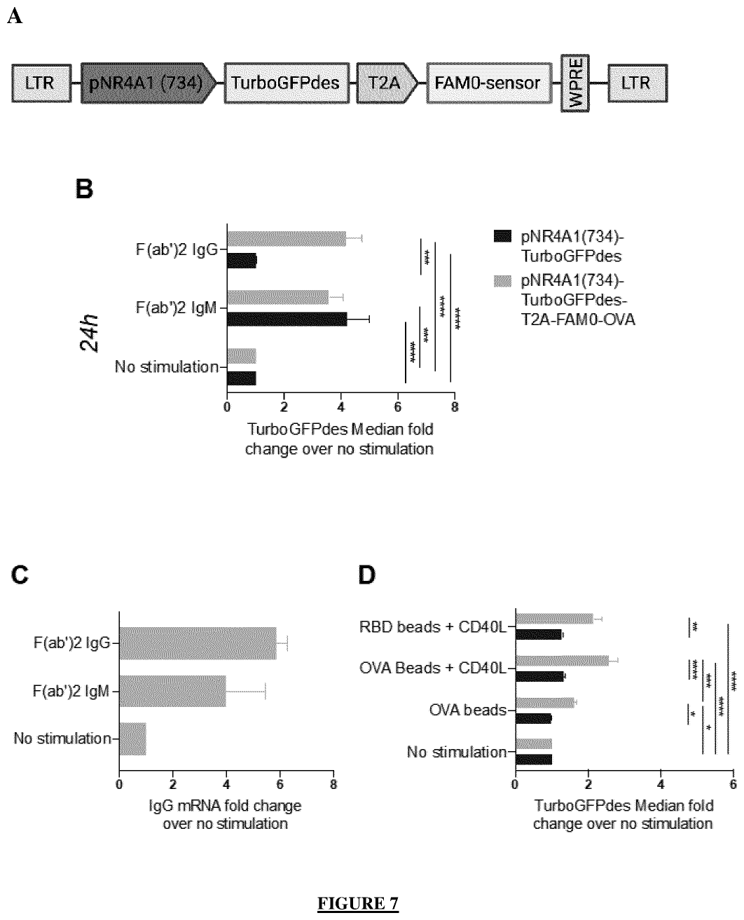

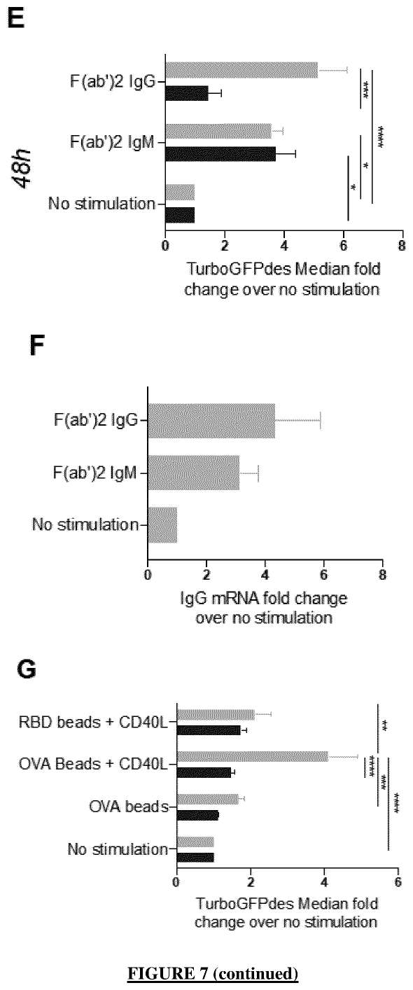

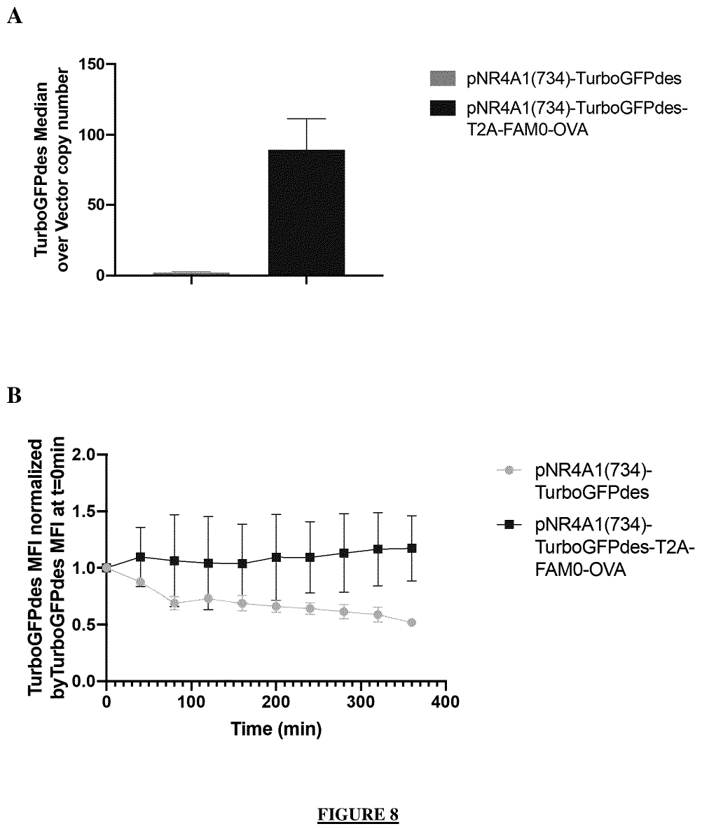

The inventors constructed a self-amplifying all-in-one-vector in which the inducible NR4A1 short promoter drove both the effector and sensor transgenes, using a T2A sequence (SEQ ID NO: 18) between the two coding sequences (FIG. 7A). Indeed, the inventors expected a leakage activity of the inducible promoter, which could induce a basal sensor expression that is required to launch the circuit upon further stimulation. Thus, upon specific sensor stimulation, the inducible promoter would be fully activated, leading to effective expression of both the effector and sensor molecules, which would amplify the synthetic circuit response, hence creating a positive feed-forward loop. Note that the insertion of the T2A motif after the effector and before the sensor ORFs resulted in addition of four amino acids fused to the TurboGFPdes marker, which may impair its destabilization and hence turn-over. This could explain the higher basal fluorescence level in cells transduced with the self-amplifying construct (pNR4A1(734)-TurboGFPdes-T2A-FAM0-OVA) as compared to cells expressing only the effector (pNR4A1(734)-TurboGFPdes) (FIG. 8).

After 24 h of stimulation with anti-IgG molecules, the inventors found that expression of both the TurboGFPdes and the sensor were upregulated in transduced cells, by 2- and 6-fold, respectively (FIGS. 7B and 7C). Importantly, the inventors showed the antigen-specific induction of this self-amplifying synthetic circuit using OVA-coated beads (FIG. 7D). Indeed, the levels of induction upon OVA-coated beads combined to CD40L stimulation was around 2 folds after 24 h and reached 4 folds after 48 h (FIGS. 7D to 7G). Similarly, the upregulation of the effector and sensor lasted after 48 h of stimulation with anti-BCR antibodies (FIGS. 7E and 7F). The effector expression remained stable after 48 h of stimulation through the IgM BCR but increased after IgG BCR stimulation as compared to 24 h of stimulation (from 4-fold after 24 h to 5-fold after 48 h), suggesting the initiation of a self-amplification (FIGS. 7E to 7G).

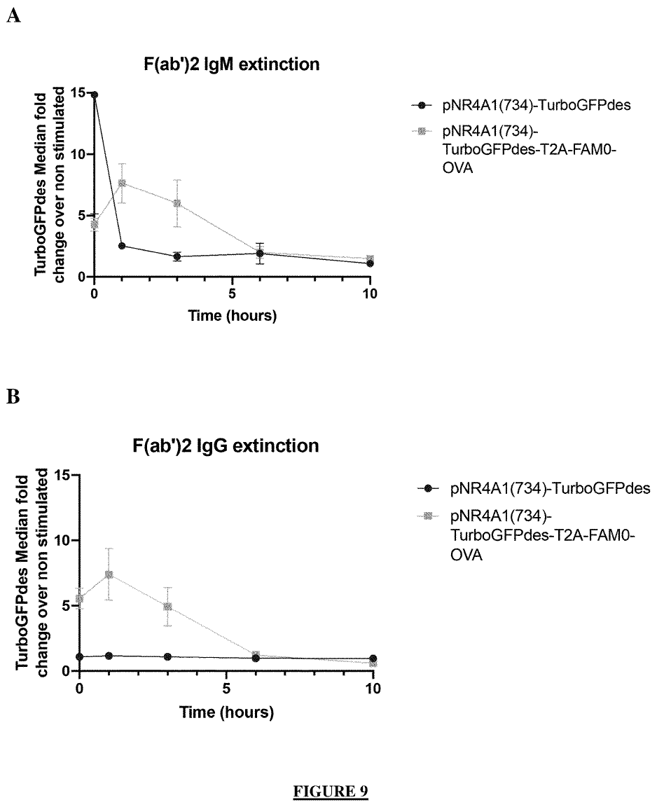

Yet, this induction was reversible as upon removal of sensor stimuli, the circuit was shut down in less than three days (FIG. 9).

### Conclusion

Overall, the inventors demonstrated that the self-regulated construct allowed specific expression of the effector upon sensor stimulation.

### REFERENCES

Sedlmayer, F.; Aubel, D.; Fussenegger, M. Synthetic Gene Circuits for the Detection, Elimination and Prevention of Disease. Nat Biomed Eng 2018, 2, 399-415, doi:10.1038/s41551-018-0215-0.
Roybal, K. T.; Lim, W. A. Synthetic Immunology: Hacking Immune Cells to Expand Their Therapeutic Capabilities. Annu. Rev. Immunol. 2017, 35, 229-253, doi:10.1146/annurev-immunol-051116-052302.
Chabannon, C.; Bouabdallah, R.; Fürst, S.; Granata, A.; Saillard, C.; Vey, N.; Mokart, D.; Fougereau, E.; Lemarie, C.; Mfarrej, B.; et al. CAR-T cells: lymphocytes exprimant un récepteur chimérique à l'antigène. La Revue de Médecine Interne 2019, 40, 545-552, doi:10.1016/j.revmed.2018.12.002.
Tittlbach, H.; Schneider, A.; Strobel, J.; Zimmermann, R.; Maas, S.; Gebhardt, B.; Rauser, G.; Mach, M.; Mackensen, A.; Winkler, T. H.; et al. GMP-Production of Purified Human B Lymphocytes for the Adoptive Transfer in Patients after Allogeneic Hematopoietic Stem Cell Transplantation. Journal of Translational Medicine 2017, 15, doi:10.1186/s12967-017-1330-5.
Ashouri, J. F.; Weiss, A. Endogenous Nur77 Is a Specific Indicator of Antigen Receptor Signaling in Human T and B Cells. J. I. 2017, 198, 657-668, doi:10.4049/jimmunol.1601301.
Cerino, A.; Bremer, C. M.; Glebe, D.; Mondelli, M. U. A Human Monoclonal Antibody against Hepatitis B Surface Antigen with Potent Neutralizing Activity. PLoS ONE 2015, 10, e0125704, doi:10.1371/journal.pone.0125704.
Dougan, S. K.; Ogata, S.; Hu, C.-C. A.; Grotenbreg, G. M.; Guillen, E.; Jaenisch, R.; Ploegh, H. L. IgG1+ Ovalbumin-Specific B-Cell Transnuclear Mice Show Class Switch Recombination in Rare Allelically Included B Cells. Proceedings of the National Academy of Sciences 2012, 109, 13739-13744, doi:10.1073/pnas.1210273109.
Fusil, F.; Calattini, S.; Amirache, F.; Mancip, J.; Costa, C.; Robbins, J. B.; Douam, F.; Lavillette, D.; Law, M.; Defrance, T.; et al. A Lentiviral Vector Allowing Physiologically Regulated Membrane-Anchored and Secreted Antibody Expression Depending on B-Cell Maturation Status. Molecular Therapy 2015, 23, 1734-1747, doi:10.1038/mt.2015.148.
Liu, Y.; Sato, H.; Hamana, M.; Moonan, N. A.; Yoneda, M.; Xia, X.; Kai, C. Construction of an Expression System for Bioactive IL-18 and Generation of Recombinant Canine Distemper Virus Expressing IL-18. J Vet Med Sci 2014, 76, 1241-1248, doi:10.1292/jvms.14-0181.
Janeway C A Jr, Travers P, Walport M, et al., Immunobiology: The Immune System in Health and Disease. 5th edition. New York: Garland Science; 2001.
Parvathaneni, Kalpana, et David W. Scott. «Engineered FVIII-Expressing Cytotoxic T Cells Target and Kill FVIII-Specific B Cells in Vitro and in Vivo». Blood Advances 2, no 18 (25 Sep. 2018): 2332 40. https://doi.org/10.1182/bloodadvances.2018018556.
Sicard, Antoine, Megan K. Levings, et David W. Scott. «Engineering Therapeutic T Cells to Suppress Alloimmune Responses Using TCR s, CAR s, or BAR s». American Journal of Transplantation 18, no 6 (June 2018): 1305 11. https://doi.org/10.1111/ajt.14747.
Ellebrecht, Christoph T., Vijay G. Bhoj, Arben Nace, Eun Jung Choi, Xuming Mao, Michael Jeffrey Cho, Giovanni Di Zenzo, et al. «Reengineering Chimeric Antigen Receptor T Cells for Targeted Therapy of Autoimmune Disease». Science 353, no 6295 (8 Jul. 2016): 179 84. https://doi.org/10.1126/science.aaf6756.
Feins, Steven, Weimin Kong, Erik F. Williams, Michael C. Milone, et Joseph A. Fraietta. «An Introduction to Chimeric Antigen Receptor (CAR) T-cell Immunotherapy for Human Cancer». American Journal of Hematology 94, no S1 (May 2019): S3 9. https://doi.org/10.1002/ajh.25418.
Schmidt, Paula, Martin J. Raftery, et Gabriele Pecher. «Engineering NK Cells for CAR Therapy-Recent Advances in Gene Transfer Methodology». Frontiers in Immunology 11 (7 Jan. 2021): 611163. https://doi.org/10.3389/fimmu.2020.611163.
Yilmaz, Ahmet, Hanwei Cui, Michael A. Caligiuri, et Jianhua Yu. «Chimeric Antigen Receptor-Engineered Natural Killer Cells for Cancer Immunotherapy». Journal of Hematology & Oncology 13, no 1 (7 Dec. 2020): 168. https://doi.org/10.1186/s13045-020-00998-9.

SEQUENCES:

SEQ ID NO: 1
GATCCATGTTGGATTGAAAAAAAATCCCCAAATCCCCATATGAGTGGACCTAC
ACAGTTCAAACGTGTTGTTCAAGGGTCAACTGTGTATGAAGACTGCTCCATCTG
TGGTGGACAGTGAGGCTTTAGAAAGAGGAGACAGGCAGGCTAATGGATGGTC
TTCATGGGTGTGGGAAAGGAGGAGTGGTCACTGGGCGCAGGCAGTTGGTCTGT
GGGACCAGTGAGGCATGGGCTGTGGCTGGCAGTAGGAAAGCGGGAGAGGCTG
AGTGCAGTGGCTCATGCCTATAATCCCAGCACTTTGGGAAGCCAAGGCAAGAG
GCTTGAGCCTGGGAGGTCCAGGCTGCAGTAAGCCATAATCATGCCACTGCATT
CTAGGCTGGGCAACAGAACAAGACCTTGTCTCAAAGACAACAAAACAAAACA
AAAACGGGGAGAGGAAAGGACCCAAGAGATGTTCCAAAAAAGAATTTCAGGA
AACAGTGAAAAGTTGCAAATGGGGCATCAGGAGCCAGGAGCAGAGATTAGGT
GGCAGGCAGGGGGTGCAGAAATGACAAGTTCTGTCTGGAACAGATGAAATTT
GAGGTGCTTGTGGGACCTTTGAGTGGGCAGAAGCGGGGGCTGGAGACAAGGG
TGTGGGAAAACTGTGATGGGGGTTGAGGGTGGAAATCGGGCCGGCAGAGCCT
CCGGGGCAGAGAAAGTCCTATGGGGTGACCAGATGAGTTGAGAACTGGGTGG
GTGGGTGCAGGCTCAATGGAAGCAGAAACTCATGGATATTGACTTACATGGGA
GAAGGGGAATGGATGGGGGTCGCAGTGGGGTGGCAGGGCTCTCTTTTCCTTGT
TTTTGTTTTTTTGCCCTTCCCTCTTCTCTTACTTTTCCGTGAGGGCTCCCCAGGCT
CAGGAGAGATCAGGGTGGAAGGGTGACGGCCAAACCAGGGAAGGCTCCAGGT
GGCTGAAGCCTGGTCTGTGCCCAGCAGGGCCCTGGCGGGCTGTTCCTCACTCC
ACCGGGCAGGTGATAACTGGTCAGAGCTGCCTCCCCACAGGTGCTGGAGGTAG
GCTGGGAGGGCCGGTGCTCCCTGATGTGGACAGGGGGAGGGGTATTGATAAG
AGGCGTGGAGAGATCCCTAGAGATGCAGTCTGTGGCCCTGGGTTCCAACCCAG
TGTGCCACCACCTGGCTGTGTGACCTTCAGCAAGTGCCATTATTTCTCTGAGCC
TGTTTGTTTATAAAATGAGGAAGAGTTGGCACAAGTTTGAAAAGATTTCTCAG
GCTCCACCCGGTTCTGAAATTCGGTAATTTCCCAACTAGGGTGCACTCCCCCTG
TAAGGGGCTGGGGAGGGGACGGTGCGAAACCAAGTTCAGCTTGTGGAGCGGA
GCCAGAGCTGTTGGCCGAGCTTGGGCCTGGCCAACGCCTGCCCTCGGGAAGGT
CCTGTGTAGGGAGACTGCCTGGAGGGACTAAGCGAGGGCTCTAACTGACGTCT
CAGGGGCAGCCTCTCAGCCTGAGACCCTGCTGGGGAAGCCGCGTCCTGTGCAC
TAGCTGCGCCTAGGGCTGAGGTGAGGGCGCAGGCTCCCCAGGGTGTGTCCGAA
TTGCCCGCCTCAGCCCGCGGCCTGTCCTGACCGCCCAGCAGCGGCAGCAGCGA
CACCCTAGGGCTCCAGGAAGGGCTTGGGAAGGTGTAAAGGCGGGGCTAGGCT
CGGAGGGAGCCGGAGGGACCGGGCGCGGTTGGCTCCCGGGAGCAACTGGAGA
GTGAGGAGATCCTCATCCGGGGAAGCCCCGCGGCCGCGTCTCTACAGCGCCCC
TTCTCGGGCTCTGGCCCTCCCGCTGGTTATTCTGGACCTGGGGGCCCCCAGCTG
GGACCCGAGTCCGGTGCGGGGAGCCTAGTGGGCCTGGGAGCTGCTATTTTTAG
CGGGCGCGGCGGGCGCGAGGAGCCTATTTATAGATCAAACAATCCGCGCTCCC
TGCGTCAATGGAACCCCGCGTGCGTCACGCGCGCAGACATTCCAGGCCCCCCC
TCCTCGCCCCGCCCCCTCGGGCTCCCCGGGCCGCACCTCCCCCTGGCCGCCTCC
CGCCGGAACCGCACCGCCCCCCGCGCCCTTGTATGGCCAAAGCTCGACGGGCG
GCCTGCGTCAGTGGCGCCCCCGCCCCTCCCCGTGCGTCACGGAGCGCTTAAGA
GGAGGGTCGGGCTCGGCCGGGGAGTCCCAGTGGCGGAGGCTACGAAACTTGG
GGGAGTGCACAGAAGAACTTCGGGAGCGCACGCGGGACCAGGGACCAGGCTG
AGACTCGGGGCGCCAGTCCGGGCAGGGGCAGCGGGA
SEQ ID NO: 2
CAGTTCAAACGTGTTGTTCAAGGGTCAACTGTGTATGAAGACTGCTCCATCTGT
GGTGGACAGTGAGGCTTTAGAAAGAGGAGACAGGCAGGCTAATGGATGGTCT
TCATGGGTGTGGGAAAGGAGGAGTGGTCACTGGGCGCAGGCAGTTGGTCTGTG
GGACCAGTGAGGCATGGGCTGTGGCTGGCAGTAGGAAAGCGGGAGAGGCTGA
GTGCAGTGGCTCATGCCTATAATCCCAGCACTTTGGGAAGCCAAGGCAAGAGG
CTTGAGCCTGGGAGGTCCAGGCTGCAGTAAGCCATAATCATGCCACTGCATTC
TAGGCTGGGCAACAGAACAAGACCTTGTCTCAAAGACAACAAAACAAAACAA
AAACGGGGAGAGGAAAGGACCCAAGAGATGTTCCAAAAAAGAATTTCAGGAA
ACAGTGAAAAGTTGCAAATGGGGCATCAGGAGCCAGGAGCAGAGATTAGGTG
GCAGGCAGGGGGTGCAGAAATGACAAGTTCTGTCTGGAACAGATGAAATTTG
AGGTGCTTGTGGGACCTTTGAGTGGGCAGAAGCGGGGGCTGGAGACAAGGGT
GTGGGAAAACTGTGATGGGGGTTGAGGGTGGAAATCGGGCCGGCAGAGCCTC
CGGGGCAGAGAAAGTCCTATGGGGTGACCAGATGAGTTGAGAACTGGGTGGG
TGGGTGCAGGCTCAATGGAAGCAGAAACTCATGGATATTGACTTACATGGGAG
AAGGGGAATGGATGGGGGTCGCAGTGGGGTGGCAGGGCTCTCTTTTCCTTGTT
TTTGTTTTTTTGCCCTTCCCTCTTCTCTTACTTTTCCGTGAGGGCTCCCCAGGCTC
AGGAGAGATCAGGGTGGAAGGGTGACGGCCAAACCAGGGAAGGCTCCAGGTG
GCTGAAGCCTGGTCTGTGCCCAGCAGGGCCCTGGCGGGCTGTTCCTCACTCCA
CCGGGCAGGTGATAACTGGTCAGAGCTGCCTCCCCACAGGTGCTGGAGGTAGG
CTGGGAGGGCCGGTGCTCCCTGATGTGGACAGGGGGAGGGGTATTGATAAGA
GGCGTGGAGAGATCCCTAGAGATGCAGTCTGTGGCCCTGGGTTCCAACCCAGT
GTGCCACCACCTGGCTGTGTGACCTTCAGCAAGTGCCATTATTTCTCTGAGCCT
GTTTGTTTATAAAATGAGGAAGAGTTGGCACAAGTTTGAAAAGATTTCTCAGG
CTCCACCCGGTTCTGAAATTCGGTAATTTCCCAACTAGGGTGCACTCCCCCTGT
AAGGGGCTGGGGAGGGGACGGTGCGAAACCAAGTTCAGCTTGTGGAGCGGAG
CCAGAGCTGTTGGCCGAGCTTGGGCCTGGCCAACGCCTGCCCTCGGGAAGGTC
CTGTGTAGGGAGACTGCCTGGAGGGACTAAGCGAGGGCTCTAACTGACGTCTC
AGGGGCAGCCTCTCAGCCTGAGACCCTGCTGGGGAAGCCGCGTCCTGTGCACT
AGCTGCGCCTAGGGCTGAGGTGAGGGCGCAGGCTCCCCAGGGTGTGTCCGAAT
TGCCCGCCTCAGCCCGCGGCCTGTCCTGACCGCCCAGCAGCGGCAGCAGCGAC
ACCCTAGGGCTCCAGGAAGGGCTTGGGAAGGTGTAAAGGCGGGGCTAGGCTC
GGAGGGAGCCGGAGGGACCGGGCGCGGTTGGCTCCCGGGAGCAACTGGAGAG
TGAGGAGATCCTCATCCGGGGAAGCCCCGCGGCCGCGTCTCTACAGCGCCCCT
TCTCGGGCTCTGGCCCTCCCGCTGGTTATTCTGGACCTGGGGGCCCCCAGCTGG
GACCCGAGTCCGGTGCGGGGAGCCTAGTGGGCCTGGGAGCTGCTATTTTTAGC
GGGCGCGGGGGCGCGAGGAGCCTATTTATAGATCAAACAATCCGCGCTCCCT
GCGTCAATGGAACCCCGCGTGCGTCACGCGCGCAGACATTCCAGGCCCCCCCT
CCTCGCCCCGCCCCCTCGGGCTCCCCGGGCCGCACCTCCCCCTGGCCGCCTCCC
GCCGGAACCGCACCGCCCCCCGCGCCCTTGTATGGCCAAAGCTCGACGGGCGG
CCTGCGTCAGTGGCGCCCCCGCCCCTCCCCGTGCGTCACGGAGCGCTTAAGAG
GAGGGTCGGGCTCGGCCGGGGAGTCCCAGTGGCGGAGGCTACGAAACTTGGG
GGAGTGCACAGAAGAACTTCGGGAGCGCACGCGGGACC
SEQ ID NO: 3
CAGGAGCAGAGATTAGGTGGCAGGCAGGGGGTGCAGAAATGACAAGTTCTGT
CTGGAACAGATGAAATTTGAGGTGCTTGTGGGACCTTTGAGTGGGCAGAAGCG
GGGGCTGGAGACAAGGGTGTGGGAAAACTGTGATGGGGGTTGAGGGTGGAAA
TCGGGCCGGCAGAGCCTCCGGGGCAGAGAAAGTCCTATGGGGTGACCAGATG
AGTTGAGAACTGGGTGGGTGGGTGCAGGCTCAATGGAAGCAGAAACTCATGG
ATATTGACTTACATGGGAGAAGGGGAATGGATGGGGGTCGCAGTGGGGTGGC
AGGGCTCTCTTTTCCTTGTTTTTGTTTTTTTGCCCTTCCCTCTTCTCTTACTTTTC
CGTGAGGGCTCCCCAGGCTCAGGAGAGATCAGGGTGGAAGGGTGACGGCCAA
ACCAGGGAAGGCTCCAGGTGGCTGAAGCCTGGTCTGTGCCCAGCAGGGCCCTG
GCGGGCTGTTCCTCACTCCACCGGGCAGGTGATAACTGGTCAGAGCTGCCTCC
CCACAGGTGCTGGAGGTAGGCTGGGAGGGCCGGTGCTCCCTGATGTGGACAGG
GGGAGGGGTATTGATAAGAGGCGTGGAGAGATCCCTAGAGATGCAGTCTGTG
GCCCTGGGTTCCAACCCAGTGTGCCACCACCTGGCTGTGTGACCTTCAGCAAG
TGCCATTATTTCTCTGAGCCTGTTTGTTTATAAAATGAGGAAGAGTTGGCACAA
GTTTGAAAAGATTTCTCAGGCTCCACCCGGTTCTGAAATTCGGTAATTTCCCAA
CTAGGGTGCACTCCCCCTGTAAGGGGCTGGGGAGGGGACGGTGCGAAACCAA
GTTCAGCTTGTGGAGCGGAGCCAGAGCTGTTGGCCGAGCTTGGGCCTGGCCAA
CGCCTGCCCTCGGGAAGGTCCTGTGTAGGGAGACTGCCTGGAGGGACTAAGCG
AGGGCTCTAACTGACGTCTCAGGGGCAGCCTCTCAGCCTGAGACCCTGCTGGG
GAAGCCGCGTCCTGTGCACTAGCTGCGCCTAGGGCTGAGGTGAGGGCGCAGGC
TCCCCAGGGTGTGTCCGAATTGCCCGCCTCAGCCCGCGGCCTGTCCTGACCGCC
CAGCAGCGGCAGCAGCGACACCCTAGGGCTCCAGGAAGGGCTTGGGAAGGTG
TAAAGGCGGGGCTAGGCTCGGAGGGAGCCGGAGGGACCGGGCGCGGTTGGCT
CCCGGGAGCAACTGGAGAGTGAGGAGATCCTCATCCGGGGAAGCCCCGCGGC
CGCGTCTCTACAGCGCCCCTTCTCGGGCTCTGGCCCTCCCGCTGGTTATTCTGG
ACCTGGGGGCCCCCAGCTGGGACCCGAGTCCGGTGCGGGGAGCCTAGTGGGC
CTGGGAGCTGCTATTTTTAGCGGGCGCGGCGGGCGCGAGGAGCCTATTTATAG
ATCAAACAATCCGCGCTCCCTGCGTCAATGGAACCCCGCGTGCGTCACGCGCG
CAGACATTCCAGGCCCCCCCTCCTCGCCCCGCCCCCTCGGGCTCCCCGGGCCGC
ACCTCCCCCTGGCCGCCTCCCGCCGGAACCGCACCGCCCCCCGCGCCCTTGTAT
GGCCAAAGCTCGACGGGCGGCCTGCGTCAGTGGCGCCCCCGCCCCTCCCCGTG
CGTCACGGAGCGCTTAAGAGGAGGGTCGGGCTCGGCCGGGGAGTCCCAGTGG
CGGAGGCTACGAAACTTGGGGGAGTGCACAGAAGAACTTCGGGAGCGCACGC
GGGACC
SEQ ID NO: 4
CAGGTGATAACTGGTCAGAGCTGCCTCCCCACAGGTGCTGGAGGTAGGCTGGG
AGGGCCGGTGCTCCCTGATGTGGACAGGGGGAGGGGTATTGATAAGAGGCGT
GGAGAGATCCCTAGAGATGCAGTCTGTGGCCCTGGGTTCCAACCCAGTGTGCC
ACCACCTGGCTGTGTGACCTTCAGCAAGTGCCATTATTTCTCTGAGCCTGTTTG
TTTATAAAATGAGGAAGAGTTGGCACAAGTTTGAAAAGATTTCTCAGGCTCCA
CCCGGTTCTGAAATTCGGTAATTTCCCAACTAGGGTGCACTCCCCCTGTAAGGG
GCTGGGGAGGGGACGGTGCGAAACCAAGTTCAGCTTGTGGAGCGGAGCCAGA
GCTGTTGGCCGAGCTTGGGCCTGGCCAACGCCTGCCCTCGGGAAGGTCCTGTG
TAGGGAGACTGCCTGGAGGGACTAAGCGAGGGCTCTAACTGACGTCTCAGGG
GCAGCCTCTCAGCCTGAGACCCTGCTGGGGAAGCCGCGTCCTGTGCACTAGCT
GCGCCTAGGGCTGAGGTGAGGGCGCAGGCTCCCCAGGGTGTGTCCGAATTGCC
CGCCTCAGCCCGCGGCCTGTCCTGACCGCCCAGCAGCGGCAGCAGCGACACCC
TAGGGCTCCAGGAAGGGCTTGGGAAGGTGTAAAGGCGGGGCTAGGCTCGGAG
GGAGCCGGAGGGACCGGGCGCGGTTGGCTCCCGGGAGCAACTGGAGAGTGAG
GAGATCCTCATCCGGGGAAGCCCCGCGGCCGCGTCTCTACAGCGCCCCTTCTC
GGGCTCTGGCCCTCCCGCTGGTTATTCTGGACCTGGGGGCCCCCAGCTGGGAC
CCGAGTCCGGTGCGGGGAGCCTAGTGGGCCTGGGAGCTGCTATTTTTAGCGGG
CGCGGCGGGCGCGAGGAGCCTATTTATAGATCAAACAATCCGCGCTCCCTGCG
TCAATGGAACCCCGCGTGCGTCACGCGCGCAGACATTCCAGGCCCCCCCTCCT
CGCCCCGCCCCCTCGGGCTCCCCGGGCCGCACCTCCCCCTGGCCGCCTCCCGCC
GGAACCGCACCGCCCCCCGCGCCCTTGTATGGCCAAAGCTCGACGGGCGGCCT
GCGTCAGTGGCGCCCCCGCCCCTCCCCGTGCGTCACGGAGCGCTTAAGAGGAG
GGTCGGGCTCGGCCGGGGAGTCCCAGTGGCGGAGGCTACGAAACTTGGGGGA
GTGCACAGAAGAACTTCGGGAGCGCACGCGGGACC
SEQ ID NO: 5
TGTGCACTAGCTGCGCCTAGGGCTGAGGTGAGGGCGCAGGCTCCCCAGGGTGT
GTCCGAATTGCCCGCCTCAGCCCGCGGCCTGTCCTGACCGCCCAGCAGCGGCA
GCAGCGACACCCTAGGGCTCCAGGAAGGGCTTGGGAAGGTGTAAAGGCGGGG
CTAGGCTCGGAGGGAGCCGGAGGGACCGGGCGCGGTTGGCTCCCGGGAGCAA
CTGGAGAGTGAGGAGATCCTCATCCGGGGAAGCCCCGCGGCCGCGTCTCTACA
GCGCCCCTTCTCGGGCTCTGGCCCTCCCGCTGGTTATTCTGGACCTGGGGGCCC
CCAGCTGGGACCCGAGTCCGGTGCGGGGAGCCTAGTGGGCCTGGGAGCTGCTA
TTTTTAGCGGGCGCGGCGGGCGCGAGGAGCCTATTTATAGATCAAACAATCCG
CGCTCCCTGCGTCAATGGAACCCCGCGTGCGTCACGCGCGCAGACATTCCAGG
CCCCCCCTCCTCGCCCCGCCCCCTCGGGCTCCCCGGGCCGCACCTCCCCCTGGC
CGCCTCCCGCCGGAACCGCACCGCCCCCCGCGCCCTTGTATGGCCAAAGCTCG
ACGGGCGGCCTGCGTCAGTGGCGCCCCCGCCCCTCCCCGTGCGTCACGGAGCG
CTTAAGAGGAGGGTCGGGCTCGGCCGGGGAGTCCCAGTGGCGGAGGCTACGA
AACTTGGGGGAGTGCACAGAAGAACTTCGGGAGCGCACGCGGGACC
SEQ ID NO: 6
GATACGAATTCCAGTTCAAACGTGTTGTTCAAGGG
SEQ ID NO: 7
GATACGAATTCCAGGAGCAGAGATTAGGTGGCA
SEQ ID NO: 8
GATACGAATTCCAGGTGATAACTGGTCAGAGCTG
SEQ ID NO: 9
GATACGAATTCTGTGCACTAGCTGCGCCTA
SEQ ID NO: 10
GATACGGATCCGGTCCCGCGTGCGCT
SEQ ID NO. 11
agcttaatacactccttaattgaagaatcgcaaaaccagcaagaaaagaatgaacaagaattattggaattagataaatgggcaagt
ttgtggaattggtttaacataacaaattggctgtggtatataaaattattcataatgatagtaggaggcttggtaggtttaagaatagttttt
gctgtactttctatagtgaatagagttaggcagggatattcaccattatcgtttcagacccacctcccaaccccgaggggacccgac
aggcccgaaggaatagaagaagaaggtggagagagagacagagacagatccattcgattagtgaacggatcggcactgcgtg
cgccaattctgcagacaaatggcagtattcatccacaattttaaaagaaaaggggggattggggggtacagtgcaggggaaagaa
tagtagacataatagcaacagacatacaaactaaagaattacaaaaacaaattacaaaaattcaaaattttcgggtttattacaggga
cagcagagatccagtttggttaattctagcatttaaatagagaaatgttctggcacctgcacttgcactggggacagcctattttg
ctagtttgttttgtttcgttttgttttgatggagagcgtatgttagtactatcgattcacacaaaaaaccaacacacagatgtaa
tgaaaataaagatattttattggcgcgccctggacaccccgcagagggtggccctaggccccctgtccgatcatgttcctgt
agtcggggatgatggtctgcttcaggtccaccaccgaggagaagatccacttcaccttgaagaaggtgacggtggcactgt
agcacacgcttaacaggaagagtgtgatgaagatggtgatggtcgtccacagcccgtccagctccccgtcctgcgcctccg
cacagctctcctccagttgcagctccggagacagggagaggctcttctgtgtgtagtggttgtgcagagcctcatgcatcac
ggagcatgagaagacgttcccctgctgccacctgctcttgtccacggtgagttttgagtagaggaagaaggagccgtcgga
gtccagcacgggaggcgtggtcttgtagttgttctccggctgcccattgctctcccactccacggcgatgtcgctgggataga
agcctttgaccaggcaggtcaggctgacttgattcttggtcagctcatcccgggatgggggcagggtgtacacctgtggttc
tcggggctgccctttggctttggagatggttttctcgatgggggctgggagggctttgttggagaccttgcacttgtactcctt
gccattcagccagtcctggtgcaggacggtgaggacgctgaccacacggtacgtgctgttgtactgctcctcccgcggcttt
gtcttggcattatgcacctccacgccgtcaacataccagttgaacttgacctcagggtcttcgtggctcacgtccaccaccac
gcatgtgacctcaggggtccgggagatcatgagggtgtccttgggttttggggggaagaggaagactgacggtcccccca
ggagttcaggtgctgggcacggtgggcatgtgtgagttttgtcacaagatttgggctcaactttcttgtccaccttggtgttgc
tgggcttgtgattcacgttgcagatgtaggtctgggtgcccaagctgctggagggcacggtcaccacgctgctgagggagt
agagtcctgaggactgtaggacagccgggaaggtgtgcacgccgctggtcagggcgcctgagttccacgacaccgtcac
cggttcggggaagtagtccttgaccaggcagcccagggccgctgtgcccccagaggtgctcttggaggagggtgccaggg
ggaagaccgatgggcccttggtgctagcTGAAGAGACAGTGACCAGAGTCCCTTGGCCCC
AGTATTTACTCCCCCTTGAACAGTAATAGACCGCAGAGTCCTCAGATGTC
AGACTGATGAGCTGAATGTAGGCTATATTGGAGGACTTGTCAACAGTCAA
TGTGGCCTTGTCTTTGAACATTTGATTGTAGTGAGTTTTTCTGTCTGAACA
ATCAATCATACCAATCCATTCAAGGCCTTGTCCAGGCCTCTGTTTCACCCA
ATTCATCCAGTAACTGGTGAAGATGTAGCCAGAAGCCTTACAGGACAGCT
TCACTGAAGCCCCAGGCCTCACCAACACAGCCccaggctgctgcagttggacctgggagtg
gactgtagctgttgctaccaaaaagaggatgatacagctccatcccatgggccctgggttggactccacgtctcccgccaa
cttgagaaggtcaaaattcaaagtctgtttcaccggtgctctcttagcccgacactctcccctattgaagctctttgtgacggg
cgaactcaggccctgatgggtgacttcgcacgcgtagactttgtgtttctcgtagtctgctttgctcagcgtcagggtgctgct
gaggctgtaggtgctgtccttgctgtcctgctctgtgacactctcctgggagttacccgattggagggcgttatccaccttcca
ctgtactttggcctctctgggatagaagttattcagcaggcacacaacagaggcagttccagatttcaactgctcatcagat
ggcgggaagatgaagacagatggtgcagccacagtCTTACGTTTGATTTCCAACTTGGTACCT
CCACCGAACGTCTGAGGAAAATGTGTAGCTTGCCAGCAATAATAAACTCC
CAAATCCTCAGCCTCCACTCTGCTGATTTTCAGTGTGAAATCCGTTCCAGA
CCCACTGCCACTGAACCTgtcagggactccagagtccagtttagacaccagatagattaggcgctttgga
gactggcctggcctctgtaacaaccaattcaaatatgtctttccatcactatctaagaggctctgacttgacttgcaagagat
ggaggctggttgtccaatggtaaccgacaaagtgagtggagtctgggtcatcacaacatcaccgttggtccgaatccaga
gcactaacagaaacaggaactgggcaggactcatcatggcggcggcgaattcaccggtacggggtggagatccgagctcg
gtaccaagcttcgtagctgtgtcctgactgagtctgactcctgcacaaagtgtgaccagcctattaataaggcttcagggcaggagg
ttgtgctctgggaacatgcaaatgagcaggggatggggcaggctgggcacagctgcacgtacgggtcaactgtaatcttggccac
ctgcctaagaggaagtggctagcttcacttctgaccctcagcaactgccaggtggcctcttggaaatccccctctgggggattccac
ccgttgggtgggagaggctgagccctatcaagttgttgcagaccaaaataatctccttaaatatcacttttgagatcagctggggtaa
acgacagcaacacaatgacaaatcattaaactattttagagattatgaaattaaaatactcagattaaaattttcctatcacagaattaag
gtactggaaaatatgtttaagtttttattaatcacattgctataggtttagatattttgtacaactgaaataaaatcacacactggcagctac
atttttgaaagttaaaaacatggtcacgaatatatcttattttaaaatcagttaatataccttaatggtatttaatgccaaattcaaagtgaat
tgatcaagccctcagtggccaggtcatgggtgtgatttttactctggctgagccgtacgaccggtgaaagggtgtggagtgctccac
ccagccctgttggaggaggatgggagcgaaagcaacagatgtgctcaaggttctgttttcagttccccaaagggtcttctccttgac
caaagcagtgtgacggttgctatgacagttgctatcttggtccctgggactctccctcataaggtagcattactttttttaatcttctcag
gcaagtaaccatgaaactcaccggtttagatcttgggtgggttaattaaccatctttatggctcgagatgttaaactgtacaagtaaag
cggccgcgactctagtccctcccaattcgatatcaagcttatcgataatcaacctctggattacaaaatttgtgaaagattgactggtat
tcttaactatgttgctccttttacgctatgtggatacgctgctttaatgcctttgtatcatgctattgcttcccgtatggctttcattttctcc
tccttgtataaatcctggttgctgtctctttatgaggagttgtggcccgttgtcaggcaacgtggcgtggtgtgcactgtgtttgctgacgc
aacccccactggttggggcattgccaccacctgtcagctcctttccgggactttcgctttccccctccctattgccacggcggaactc
atcgccgcctgccttgcccgctgctggacaggggctcggctgttgggcactgacaattccgtggtgttgtcggggaaatcatcgtc
ctttccttggctgctcgcctgtgttgccacctgcattctgcgcgggacgtccttctgctacgtcccttcggccctcaatccagcggacc
ttccttcccgcggcctgctgccggctctgcggcctcttccgcgtcttcgccttcgccctcagacgagtcggatctccctttgggccgc
ctccccgcatcgataccgtcgacctcgatcgagacctagaaaaacatggagcaatcacaagtagcaatacagcagctaccaatgc
tgattgtgcctggctagaagcacaagaggaggaggaggtgggttttccagtcacacctcaggtacctttaagaccaatgacttaca
aggcagctgtagatcttagccactttttaaaagaaaaggggggactggaagggctaattcactcccaacgaagacaagatatcctt
gatctgtggatctaccacacacaaggctacttccctgattggcagaactacacaccagggccagggatcagatatccactgaccttt
ggatggtgctacaagctagtaccagttgagcaagagaaggtagaagaagccaatgaaggagagaacacccgcttgttacaccct
gtgagcctgcatgggatggatgacccggagagagaagtattagagtggaggtttgacagccgcctagcatttcatcacatggccc
gagagctgcatccggactgtactgggtctctctggttagaccagatctgagcctgggagctctctggctaactagggaacccactg
cttaagcctcaataaagcttgccttgagtgcttcaagtagtgtgtgcccgtctgttgtgtgactctggtaactagagatccctcagacc
cttttagtcagtgtggaaaatctctagcagggcccgtttaaacccgctgatcagcctcgactgtgccttctagttgccagccatctgtt
gtttgcccctcccccgtgccttccttgaccctggaaggtgccactcccactgtcctttcctaataaaatgaggaaattgcatcgcattgt
ctgagtaggtgtcattctattctggggggtggggtggggcaggacagcaagggggaggattgggaagacaatagcaggcatgct
ggggatgcggtgggctctatggcttctgaggcggaaagaaccagctggggctctagggggtatccccacgcgccctgtagcgg
cgcattaagcgcggcgggtgtggtggttacgcgcagcgtgaccgctacacttgccagcgccctagcgcccgctcctttcgctttctt
cccttcctttctcgccacgttcgccggctttccccgtcaagctctaaatcgggggctccctttagggttccgatttagtgctttacggca
cctcgaccccaaaaaacttgattagggtgatggttcacgtagtgggccatcgccctgatagacggtttttcgccctttgacgttggag
tccacgttctttaatagtggactcttgttccaaactggaacaacactcaaccctatctcggtctattcttttgatttataagggattttgccg
atttcggcctattggttaaaaaatgagctgatttaacaaaaatttaacgcgaattaattctgtggaatgtgtgtcagttagggtgtggaa
agtccccaggctccccagcaggcagaagtatgcaaagcatgcatctcaattagtcagcaaccaggtgtggaaagtccccaggctc
cccagcaggcagaagtatgcaaagcatgcatctcaattagtcagcaaccatagtcccgcccctaactccgcccatcccgccccta
actccgcccagttccgcccattctccgccccatggctgactaattttttttatttatgcagaggccgaggccgcctctgcctctgagct
attccagaagtagtgaggaggcttttttggaggcctaggcttttgcaaaaagctcccgggagcttgtatatccattttcggatctgatc
agcacgtgttgacaattaatcatcggcatagtatatcggcatagtataatacgacaaggtgaggaactaaaccatggccaagttgac
cagtgccgttccggtgctcaccgcgcgcgacgtcgccggagcggtcgagttctggaccgaccggctcgggttctcccgggactt
cgtggaggacgacttcgccggtgtggtccgggacgacgtgaccctgttcatcagcgcggtccaggaccaggtggtgccggaca
acaccctggcctgggtgtgggtgcgcggcctggacgagctgtacgccgagtggtcggaggtcgtgtccacgaacttccgggac
gcctccgggccggccatgaccgagatcggcgagcagccgtgggggcgggagttcgccctgcgcgacccggccggcaactgc
gtgcacttcgtggccgaggagcaggactgacacgtgctacgagatttcgattccaccgccgccttctatgaaaggttgggcttcgg
aatcgttttccgggacgccggctggatgatcctccagcgcggggatctcatgctggagttcttcgcccaccccaacttgtttattgca
gcttataatggttacaaataaagcaatagcatcacaaatttcacaaataaagcatttttttcactgcattctagttgtggtttgtccaaact
catcaatgtatcttatcatgtctgtataccgtcgacctctagctagagcttggcgtaatcatggtcatagctgtttcctgtgtgaaattgtt
atccgctcacaattccacacaacatacgagccggaagcataaagtgtaaagcctggggtgcctaatgagtgagctaactcacatta
attgcgttgcgctcactgcccgctttccagtcgggaaacctgtcgtgccagctgcattaatgaatcggccaacgcgcggggagag
gcggtttgcgtattgggcgctcttccgcttcctcgctcactgactcgctgcgctcggtcgttcggctgcggcgagcggtatcagctc
actcaaaggcggtaatacggttatccacagaatcaggggataacgcaggaaagaacatgtgagcaaaaggccagcaaaaggcc
aggaaccgtaaaaaggccgcgttgctggcgtttttccataggctccgcccccctgacgagcatcacaaaaatcgacgctcaagtc
agaggtggcgaaacccgacaggactataaagataccaggcgtttccccctggaagctccctcgtgcgctctcctgttccgaccctg
ccgcttaccggatacctgtccgcctttctcccttcgggaagcgtggcgctttctcatagctcacgctgtaggtatctcagttcggtgta
ggtcgttcgctccaagctgggctgtgtgcacgaaccccccgttcagcccgaccgctgcgccttatccggtaactatcgtcttgagtc
caacccggtaagacacgacttatcgccactggcagcagccactggtaacaggattagcagagcgaggtatgtaggcggtgctac
agagttcttgaagtggtggcctaactacggctacactagaagaacagtatttggtatctgcgctctgctgaagccagttaccttcgga
aaaagagttggtagctcttgatccggcaaacaaaccaccgctggtagcggtggtttttttgtttgcaagcagcagattacgcgcaga
aaaaaaggatctcaagaagatcctttgatcttttctacggggtctgacgctcagtggaacgaaaactcacgttaagggattttggtcat
gagattatcaaaaaggatcttcacctagatccttttaaattaaaaatgaagttttaaatcaatctaaagtatatatgagtaaacttggtctg
acagttaccaatgcttaatcagtgaggcacctatctcagcgatctgtctatttcgttcatccatagttgcctgactccccgtcgtgtagat
aactacgatacgggagggcttaccatctggccccagtgctgcaatgataccgcgagacccacgctcaccggctccagatttatca
gcaataaaccagccagccggaagggccgagcgcagaagtggtcctgcaactttatccgcctccatccagtctattaattgttgccg
ggaagctagagtaagtagttcgccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaaaaagcggttagctccttcgg
tcctccgatcgttgtcagaagtaagttggccgcagtgttatcactcatggttatggcagcactgcataattctcttactgtcatgccatc
cgtaagatgcttttctgtgactggtgagtactcaaccaagtcattctgagaatagtgtatgcggcgaccgagttgctcttgcccggcgt
caatacgggataataccgcgccacatagcagaactttaaaagtgctcatcattggaaaacgttcttcggggcgaaaactctcaagg
atcttaccgctgttgagatccagttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactcatactcttcctttttcaa
tattattgaagcatttatcagggttattgtctcatgagcggatacatatttgaatgtatttagaaaaataaacaaataggggttccgcgca
catttccccgaaaagtgccacctgacgtcgacggatcgggagatctcccgatcccctatggtgcactctcagtacaatctgctctga
tgccgcatagttaagccagtatctgctccctgcttgtgtgttggaggtcgctgagtagtgcgcgagcaaaatttaagctacaacaag
gcaaggcttgaccgacaattgcatgaagaatctgcttagggttaggcgttttgcgctgcttcgcgatgtacgggccagatatacgcg
ttgacattgattattgactagttattaatagtaatcaattacggggtcattagttcatagcccatatatggagttccgcgttacataacttac
ggtaaatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagtacgcc
ccctattgacgtcaatgacggtaaatggcccgcctggcattatgcccagtacatgaccttatgggactttcctacttggcagtacatct
acgtattagtcatcgctattaccatggtgatgcggttttggcagtacatcaatgggcgtggatagcggtttgactcacggggatttcca
agtctccaccccattgacgtcaatgggagtttgttttggcaccaaaatcaacgggactttccaaaatgtcgtaacaactccgccccatt
gacgcaaatgggcggtaggcgtgtacggtgggaggtctatataagcagcgcgttttgcctgtactgggtctctctggttagaccag
atctgagcctgggagctctctggctaactagggaacccactgcttaagcctcaataaagcttgccttgagtgcttcaagtagtgtgtg
cccgtctgttgtgtgactctggtaactagagatccctcagacccttttagtcagtgtggaaaatctctagcagtggcgcccgaacagg
gacttgaaagcgaaagggaaaccagaggagctctctcgacgcaggactcggcttgctgaagcgcgcacggcaagaggcgagg
ggcggcgactggtgagtacgccaaaaattttgactagcggaggctagaaggagagagatgggtgcgagagcgtcagtattaagc
gggggagaattagatcgcgatgggaaaaaattcggttaaggccagggggaaagaaaaaatataaattaaaacatatagtatgggc
aagcagggagctagaacgattcgcagttaatcctggcctgttagaaacatcagaaggctgtagacaaatactgggacagctacaa
ccatcccttcagacaggatcagaagaacttagatcattatataatacagtagcaaccctctattgtgtgcatcaaaggatagagataa
aagacaccaaggaagctttagacaagatagaggaagagcaaaacaaaagtaagaccaccgcacagcaagcggccgctgatctt
cagacctggaggaggagatatgagggacaattggagaagtgaattatataaatataaagtagtaaaaattgaaccattaggagtag
cacccaccaaggcaaagagaagagtggtgcagagagaaaaaagagcagtgggaataggagctttgttccttgggttcttgggag
cagcaggaagcactatgggcgcagcgtcaatgacgctgacggtacaggccagacaattattgtctggtatagtgcagcagcaga
acaatttgctgagggctattgaggcgcaacagcatctgttgcaactcacagtctggggcatcaagcagctccaggcaagaatcctg
gctgtggaaagatacctaaaggatcaacagctcctggggatttggggttgctctggaaaactcatttgcaccactgctgtgccttgg
aatgctagttggagtaataaatctctggaacagatttggaatcacacgacctggatggagtgggacagagaaattaacaattacaca
SEQ ID NO: 12
TGT GTG CCC GTC TGT TGT GT
SEQ ID NO: 13
GAG TCC TGC GTC GAG AGA GC

SEQ ID NO: 14
CAG TGG CGC CCG AAC AGG GA
SEQ ID NO: 15
TCC GTG TGG ATC GGC GGC TCC A
SEQ ID NO: 16
CTG CTT GCT GAT CCA CAT CTG
SEQ ID NO: 17
CCT GGC CTC GCT GTC CAC CTT CCA
SEQ ID NO: 18
GAGGGCAGAGGAAGTCTTCTAACATGCGGTGACGTGGAGGAGAATCCCGGCC
CT
SEQ ID NO: 19
GATCGAATTCCAGGAAGGGCTTGGGAAGGTGTAAAG
SEQ ID NO: 20
GATCGAATTCAGCTGGGACCCGAGTCCGGT
SEQ ID NO: 21
GATCGAATTCTTGTATGGCCAAAGCTCGACGGG
SEQ ID NO: 22
CAGGAAGGGCTTGGGAAGGTGTAAAGGCGGGGCTAGGCTCGGAGGGAGCCGG
AGGGACCGGGCGCGGTTGGCTCCCGGGAGCAACTGGAGAGTGAGGAGATCCT
CATCCGGGGAAGCCCCGCGGCCGCGTCTCTACAGCGCCCCTTCTCGGGCTCTG
GCCCTCCCGCTGGTTATTCTGGACCTGGGGGCCCCCAGCTGGGACCCGAGTCC
GGTGCGGGGAGCCTAGTGGGCCTGGGAGCTGCTATTTTTAGCGGGCGCGGCGG
GCGCGAGGAGCCTATTTATAGATCAAACAATCCGCGCTCCCTGCGTCAATGGA
ACCCCGCGTGCGTCACGCGCGCAGACATTCCAGGCCCCCCCTCCTCGCCCCGC
CCCCTCGGGCTCCCCGGGCCGCACCTCCCCCTGGCCGCCTCCCGCCGGAACCG
CACCGCCCCCCGCGCCCTTGTATGGCCAAAGCTCGACGGGCGGCCTGCGTCAG
TGGCGCCCCCGCCCCTCCCCGTGCGTCACGGAGCGCTTAAGAGGAGGGTCGGG
CTCGGCCGGGGAGTCCCAGTGGCGGAGGCTACGAAACTTGGGGGAGTGCACA
GAAGAACTTCGGGAGCGCACGCGGGACC
SEQ ID NO: 23
AGCTGGGACCCGAGTCCGGTGCGGGGAGCCTAGTGGGCCTGGGAGCTGCTATT
TTTAGCGGGCGCGGCGGGCGCGAGGAGCCTATTTATAGATCAAACAATCCGCG
CTCCCTGCGTCAATGGAACCCCGCGTGCGTCACGCGCGCAGACATTCCAGGCC
CCCCCTCCTCGCCCCGCCCCCTCGGGCTCCCCGGGCCGCACCTCCCCCTGGCCG
CCTCCCGCCGGAACCGCACCGCCCCCCGCGCCCTTGTATGGCCAAAGCTCGAC
GGGCGGCCTGCGTCAGTGGCGCCCCCGCCCCTCCCCGTGCGTCACGGAGCGCT
TAAGAGGAGGGTCGGGCTCGGCCGGGGAGTCCCAGTGGCGGAGGCTACGAAA
CTTGGGGGAGTGCACAGAAGAACTTCGGGAGCGCACGCGGGACC

1. A nucleic acid system comprising:
(i) a sensor component comprising a sequence encoding
(a) at least one extracellular ligand recognition domain;
(b) a transmembrane domain of a B-cell receptor (BCR) or of a T-cell receptor (TCR); and
(c) optionally a signaling domain that controls cell activation of a B-cell receptor or of a T-cell receptor; and
(ii) a transducer/effector component comprising a sequence encoding
(d) the pNR4A1 promoter consisting of SEQ ID NO: 1, or a functional fragment thereof; and
(e) at least one effector protein of interest.

2. The nucleic acid system according to claim 1 wherein the transducer/effector component comprises (d) a functional fragment of the pNR4A1 promoter.

3. The nucleic acid system according to claim 1, wherein the functional fragment of the pNR4A1 promoter has a length from 200 bp to 2210 bp, or from 500 bp to 2210 bp.

4. The nucleic acid system according to claim 1, wherein the functional fragment of the pNR4A1 promoter has a nucleic acid sequence selected from the group consisting of SEQ ID NO:2, SEQ ID NO:3, SEQ ID NO:4, SEQ ID NO:5, SEQ ID NO: 22 and SEQ ID NO: 23.

5. The nucleic acid system according to claim 1, wherein the extracellular ligand recognition domain (a), the transmembrane domain (b) and, if present, the signaling domain (c) form a B-cell receptor, a T-cell receptor, a chimeric immune receptor (CIR), a CAR-NK cell, a B-cell antibody receptor (BAR) or a chimeric autoantibody receptor T (CAAR-T) cell.

6. The nucleic acid system according to claim 1, wherein the extracellular ligand recognition domain (a) comprises at least one ligand binding fragment which binds to a ligand of interest.

7. The nucleic acid system according to claim 1, wherein the antigen-binding domain is selected from antigen-binding domains of antibodies, Fd fragments, single domain antibodies (sdAb), complementary determining regions (CDR), Fv fragments, single chain FVs (scFV), dsFvs, and sc(Fv)2; antigen-binding domains of antibody mimetics aptamers, and mixtures thereof.

8. The nucleic acid system according to claim 6, wherein the ligand of interest is selected from one or more tumor antigens, one or more self-antigens, one or more allo-antigens, one or more viral antigens, one or more bacterial antigens, one or more allergen antigens, or one or more markers of neurological disorders.

9. The nucleic acid system according to claim 1, wherein the at least one effector protein of interest is an immunostimulatory protein or an immunosuppressant protein.

10. The nucleic acid system according to claim 1, wherein the at least one effector protein of interest is selected from pro-inflammatory cytokines; anti-inflammatory cytokines; co-stimulation molecules; and inhibitor molecules.

11. The nucleic acid system according to claim 1, further comprising (iii) an ORF separating sequence between the (i) sensor component and the (ii) transducer/effector components and wherein said (i) sensor component and (ii) transducer/effector component are both under the control of the pNR4A1 promoter consisting of SEQ ID NO: 1, or a functional fragment thereof.

12. A vector comprising a nucleic acid system according to claim 1.

13. The vector according to claim 12 that is a retroviral vector.

14. A kit comprising:
a first vector comprising the sensor component of the nucleic acid system according to claim 1; and
a second vector comprising the transducer/effector component of the nucleic acid system according to claim 1.

15. A cell which has been transformed by a nucleic acid system according to claim 1, by a vector comprising the nucleic acid system of claim 1, or by a kit comprising a first vector comprising the sensor component of the nucleic acid system according to claim 1 and a second vector comprising the transducer/effector component of the nucleic acid system according to claim 1.

16. A pharmaceutical composition comprising a nucleic acid system according to claim 1, a vector comprising the nucleic acid system of claim 1, a kit comprising a first vector comprising the sensor component of the nucleic acid system according to claim 1 and a second vector comprising the transducer/effector component of the nucleic acid system according to claim 1, or a cell which has been transformed by the nucleic acid system, the vector or the kit, and a pharmaceutically acceptable vehicle.

17. A method for preventing and/or treating a tumor, an immune disorder, a transplantation rejection, an allergy, a neurological disorder, and/or an infectious disease, comprising at least a step of administering to an individual in need thereof a nucleic acid system according to claim 1, a vector comprising the nucleic acid system of claim 1, a kit comprising a first vector comprising the sensor component of the nucleic acid system according to claim 1 and a second vector comprising the transducer/effector component of the nucleic acid system according to claim 1, a cell which has been transformed by the nucleic acid system, the vector or the kit, or a pharmaceutical composition comprising the nucleic acid system, the vector, the kit or the cell.

18. (canceled)
